(19) United States
(12) Patent Application Publication
(10) Pub. No.: US 2026/0102658 A1
(43) Pub. Date: Apr. 16, 2026

(54) ARTIFICIAL FITNESS TRAINER AND YOGA INSTRUCTOR SYSTEM

(71) Applicants: Alexander Misharin, North Wales, PA (US); Andrei Podlesnyi, Moscow (RU)

(72) Inventors: Alexander Misharin, North Wales, PA (US); Andrei Podlesnyi, Moscow (RU)

(21) Appl. No.: 19/356,115

(22) Filed: Oct. 11, 2025

Related U.S. Application Data
(60) Provisional application No. 63/706,650, filed on Oct. 12, 2024.

(51) Int. Cl. A63B 24/00; A63B 71/06; G06V 10/80; G06V 40/20

(52) U.S. Cl. CPC A63B 24/0075; A63B 71/0622; A63B 2024/0068; A63B 2071/063; A63B 2071/0694; A63B 2220/05; A63B 2220/806; A63B 2225/20; G06V 10/806; G06V 40/23

(57) ABSTRACT

A system and method provide real-time coaching for exercise and yoga. A client application on a device captures images or video via cameras and may capture audio via a microphone. An artificial intelligence agent estimates pose, evaluates movement sequences and breathing, classifies deviations from a reference, and returns a multi-segment response that includes brief commands for in-motion cues, detailed instructions for learning, and additional information. A dynamic response selector on the client selects segment(s) based on application state and user actions and renders overlays on a device display or augmented-reality headset. Optional wearable sensors and multiple cameras feed a fusion module to increase fidelity. Processing is partitioned between on-device and cloud components with anonymization, encryption, and caching to reduce latency and protect privacy.

### CROSS-REFERENCES TO RELATED APPLICATIONS

This application claims the benefit of U.S. provisional patent application No. 63/706,650, filed on Oct. 12, 2024 and entitled “ARTIFICIAL FITNESS TRAINER AND YOGA INSTRUCTOR SYSTEM”. The U.S. provisional Ser. No. 63/706,650 is incorporated herein by reference in its entirety.

### FIELD OF THE INVENTION

The disclosure relates to computational systems and methods for real-time exercise and yoga coaching using client applications and artificial intelligence agents to analyze user movement, posture, timing, and breathing, and to deliver adaptive corrections and instruction.

### BACKGROUND

Effective physical training typically requires expert observation and individualized feedback. Access to such instruction is limited by cost and availability, and remote content lacks personalization. Automated guidance often fails to respond to real-time context or to respect privacy constraints. There remains a need for systems that transform commodity devices into capable trainers, combining low-latency on-device analysis with cloud-scale modeling, and that present the right level of guidance at the right time.

Traditional fitness training and yoga instruction often require the presence of a physical human trainer or instructor to provide real-time feedback, correct posture, and offer personalized advice. This can be costly, time-consuming, and inaccessible for many individuals. The need exists for a technology-based solution that can offer similar benefits remotely and affordably.

### BRIEF SUMMARY

In certain embodiments, a client application captures images, video, and/or audio describing a user's body and environment, and transmits signals to an artificial intelligence (AI) agent that estimates pose keypoints, evaluates movement sequences, analyzes breathing, and scores deviations from one or more reference models. The agent produces a response that includes multiple segments, such as concise commands suitable for in-motion correction, detailed instructional narratives appropriate for learning phases, and supplemental information. A Dynamic Response Selector (DRS) in the client selects which segment(s) to present based on application state and user actions. Optional wearable sensors and multiple cameras enhance accuracy through fusion, and security features including encryption, anonymization, and caching protect user data while distributing computation across device and cloud.

The disclosed systems can deliver immediate safety-critical corrections during practice, amplify retention through post-session instruction, and reduce bandwidth by bundling multi-segment responses.

The present patent document provides systems and methods for an artificial fitness trainer or yoga instructor that utilizes a mobile application (app) or computer program to deliver real-time feedback and instructions to users based on their physical activity. A system according to the disclosed technology comprises a video camera, a computing device such as a processor, mobile phone, or tablet, and may include a microphone and/or speaker for enhanced interaction. The computing device is configured to host a client application that is configured to interact with or incorporate an artificial intelligence (AI) agent or software, or a machine learning (ML)-based system or software. This AI agent is configured to process the data captured by the camera and the microphone, providing personalized feedback and instructions to the user in real time.

In certain embodiments, a client application (10) operating on a client device (110) captures images and/or audio via camera(s) (120/121-126) and microphone (130) and exchanges signals with an AI agent (150) over a network (160).

The agent estimates pose keypoints (151), evaluates movement sequences (152), analyzes breathing (153), classifies errors (154), and generates multi-segment responses (471-473) comprising brief commands, detailed instructions, and supplemental information.

A Dynamic Response Selector (DRS) (250/474) chooses which segment(s) to present via a presentation layer (270), including overlays (360) on a display (111) or AR/VR glasses (190).

Optional wearables (180) and multiple cameras (121-126) feed a fusion module (127) to improve fidelity. On-device processing (481) handles privacy redaction (485), text-to-speech, and overlays; cloud processing (482) handles model-heavy analysis with encryption (500) and caching (486).

### BRIEF DESCRIPTION OF THE DRAWINGS

FIG. 1 shows a main screen of the client application with a primary menu control.

FIG. 2 shows an expanded main menu panel displaying navigation options.

FIG. 3 shows an “Add-a-Pose” view listing saved references with thumbnails.

FIG. 4 shows a Poses/Asanas selector with options for gallery comparison and live comparison.

FIG. 5 shows a Settings view including user-adjustable controls for operation.

FIG. 6 shows an image or album picker interface.

FIG. 7 shows an example reference-pose image with an instruction panel.

FIG. 8 shows an example user-pose image with generated instructions.

FIG. 9 shows a live camera viewfinder with a camera selection control.

FIG. 10 shows a real-time guidance screen with a compact brief panel.

FIG. 11 shows a system-level architecture including a client device, networked AI agent, model/pose store, optional wearables, and an external camera.

FIG. 12 shows client-side modules including capture, pre-processing, request generation, state monitoring, a dynamic response selector, response handling, and presentation.

FIG. 13 shows an AI-agent pipeline including pose estimation, movement analysis, breathing analysis, error classification, feedback generation, and response segmentation.

FIG. 14 shows a selection framework that bundles response segments and uses phase-aware context for dynamic presentation.

FIG. 15 shows a pose-comparison and overlay pipeline from reference and user frames through measurement and instruction to overlay rendering.

FIG. 16 shows multi-sensor configurations and fusion across visual and depth/IR modalities to improve robustness.

FIG. 17 shows edge/cloud partitioning with privacy, compression, encryption, and caching in the processing flow.

FIG. 18 shows privacy filtering, transport security, caching, and related data-lifecycle operations.

FIG. 19 shows an end-to-end method flow from mode selection and capture through secure analysis, selection, and presentation.

FIG. 20 shows dynamic response selection from an arriving server response using application state and selection logic.

FIG. 21 shows movement-sequence analysis with phase segmentation, timing comparison, breathing synchronization, severity classification, and feedback generation.

FIG. 22 shows quality and context checks that can trigger additional capture, integrate new data, perform fusion, and return improved analysis.

FIG. 23 shows security and processing flows spanning on-device handling, encrypted transport, cloud processing, and caching.

FIG. 24 shows an augmented-reality/heads-up display embodiment with overlays anchored in world, device, and body frames.

FIG. 25 shows a coach co-pilot embodiment in which AI-generated guidance is optionally reviewed or reshaped before presentation.

FIG. 26 shows a handset hardware block diagram of representative device components for the client.

FIG. 27 shows wearable and auxiliary sensors communicating with the client device and contributing to fusion.

FIG. 28 shows representative network topologies, including direct and intermediated paths.

### DETAILED DESCRIPTION

Examples and embodiments are illustrative. The drawings and description include examples and embodiments to explain, not to limit, the disclosed technology. Unless expressly stated otherwise, no single feature, step, or configuration is required in every embodiment; features described in connection with a particular embodiment may be combined with, separated from, substituted for, or omitted relative to features in other embodiments. Alternative implementations that perform substantially the same function or achieve substantially the same result may be used.

No “best” is implied. References to “example”, “illustrative”, “sample”, “non-limiting”, “preferred”, or similar terms do not indicate that an embodiment is required, optimal, or superior. Absence of a feature in an example does not disclaim that feature.

Variations and equivalents. Unless the context dictates otherwise, operations may be performed in different orders or in parallel; components may be implemented in hardware, software, firmware, services, or combinations; functions described for one component may be performed by others; and distributed or partitioned implementations (e.g., edge/cloud, intermediary servers) are contemplated. Modalities (e.g., images, video, audio, depth/IR/LiDAR/ToF/thermal, wearables), model families (e.g., keypoint-based, keypoint-optional, vision-language), and orchestration choices (single-agent or multi-agent) are implementation details.

Media, formats, and data handling. Unless otherwise stated, images, clips, enhanced media, overlay directives, bundles, and metadata may be transmitted, cached, transformed, redacted, or encrypted as described, with variants and sub-combinations permitted.

### Definitions and Core Concepts

General interpretation rules. As used in this document, unless the context indicates otherwise: (i) “a” or “an” means one or more; (ii) “or” is inclusive (A, B, or both); (iii) “including,” “such as,” and similar terms are non-limiting; (iv) the terms “first,” “second,” etc. are merely labels and do not imply order or priority; (v) terms of degree (e.g., “approximately,” “about,” “near,” “substantially,” “real-time”) are understood as a person of ordinary skill in the art would, in view of the measurement tolerances and context disclosed herein; and (vi) unless expressly stated otherwise, recitations using open transitional language (e.g., “comprising”) do not exclude additional elements or steps. Furthermore, unless expressly stated otherwise, (i) a recited range (e.g., “from X to Y,” “between X and Y”) includes all intermediate values and all sub-ranges within the stated bounds, with endpoints included unless exclusion is clear from context; and (ii) a recited list of items (e.g., “A, B, C” or “A, B, and C”) supports any of A, B, or C individually and any combination or sub-combination thereof. These principles apply to the entire document and all claims.

Phase; Phase Boundary; Phase-Aware Features (610-615). A phase is a semantically meaningful, time-bounded portion of a movement sequence (e.g., ingress, hold, egress). A phase boundary is a point/interval indicating transition between phases. Phase-aware features may include: phase segmentation (610), boundary detection (611), transition duration (612), timing vs. reference (613), breathing-sync checks (614), and severity labels (615). Signals may come from pose trajectories, cadence/timing, and/or breathing cues.

Dynamic Response Selector (DRS 250) and Selection Logic (474). The DRS is a client-side selector that uses selection logic (474) (e.g., rule-based, machine learning (ML), or hybrid) to choose which response segment(s) to surface and when. Inputs may include: time-to-boundary (e.g., time to predicted or estimated upcoming boundary; upcoming boundary predictions/estimates can be made by, e.g., the client app or an AI agent; upcoming boundary estimates/predictions can be included into AI agent responses (e.g., as a response segment)), transition duration (612), timing variance vs. reference (613), breathing sync (614), severity (615), quality/context (620), application state (240), user actions, and runtime constraints (e.g., bandwidth/battery/confidence).

Multi-Segment Bundle (471-473). A multi-segment bundle is any output comprising two or more distinct, self-contained segments (e.g., brief commands (471), detailed instructions (472), additional information (473)). A bundle can be produced, e.g., by an AI agent and/or by the client application and delivered in one or multiple messages; segment labels and contents are illustrative.

Model/Pose Store (170) and Reference Item. The model/pose store (170) is a logical repository accessible to the client (200) and/or agent (150), containing (i) reference items (e.g., reference images/videos, timing templates, parametric constraints) with metadata (e.g., names, keypoints/angles, phases, timing, breathing cues), and/or (ii) model assets/templates for analysis and comparison. Reference items are the materials used as targets for comparison or guidance; they may be user-curated (“Add-a-Pose”) and/or system-provided.

User-Curated Library (“Add-a-Pose”) and Objective Overlays (360). Users may create/select reference items that are stored in 170. The system can associate objective overlays (360) (e.g., lines, angles, vectors, alignment guides) and/or model-driven criteria with those references for use during analysis or review. “Add-a-Pose” is an example label and non-limiting.

Overlay Directive; Presentation Layer (270); Coordinate Frames (191/192/193). An overlay directive is data/instructions sufficient for a client to render visual guides (lines, arcs, markers, labels, angle glyphs, arrows), anchored in one or more frames: world (191), device (192), body (193); device-anchored HUD (heads-up display) elements live in HUD/screen space. The presentation layer (270) schedules/renders text/audio/images/video/overlays (including augmented reality (AR)/HUD) per DRS timing/comfort policies.

Application State (240) and Quality/Context Signals (620). Application state (240) denotes client context (e.g., practicing, hold, review, idle/explore) and runtime conditions. Quality/context (620) includes capture and runtime indicators (e.g., lighting/backlight/exposure, framing/field of view (FOV)/distance, stability, bandwidth/battery, confidence) used to steer capture, analysis, and presentation.

Client context definitions: Practicing is the live, in-motion state while capture/streaming is active; the DRS favors concise, real-time cues and minimal overlays to land before phase boundaries. Hold is a steady or paused window (e.g., while a pose is maintained) in which richer guidance, overlays, and breath/cadence resynchronization can be surfaced. Review is the analysis/browse state for captured media (including gallery comparison), emphasizing detailed instruction and side-by-side visuals, with brief items as compact recaps. Idle/Explore covers reference browsing, Add-a-Pose, settings, and how-to content, where supplemental information is foregrounded and time-critical prompts are suppressed. These states are tracked by the state monitor (240) and consumed by the DRS (250)/selection logic (474) to choose which response segment (e.g., brief 471/detailed 472/additional 473) to present.

Fusion Module (127); Multi-Sensor Fusion (121-126); Pose Fidelity in Hard Conditions. The fusion module (127) temporally/spatially aligns and combines one or more modalities (e.g., RGB (red-green-blue camera; 121), depth (122), IR (infrared; 123), LiDAR (light detection and ranging; 124), ToF (time of flight; 125), thermal (126), and optionally wearables (180)) to increase confidence, disambiguate occlusions, and stabilize geometry. Pose fidelity in hard conditions refers to improved estimation and overlays under low light, clutter, occlusion, fast motion, or large spaces via such fusion.

External Viewpoints (195) and Wearables (180). Auxiliary cameras (including external ones (e.g., on a drone or in a robotic system (e.g., a humanoid robot) or in a smart home appliance)) and wearables (e.g., IMU, respiration) can be fused to boost coverage, timing, and breathing estimation, subject to the same privacy/transport controls.

Enhanced Image/Video. An enhanced image or enhanced video is any representation derived from captured media that may be normalized, augmented with derived channels/metadata (e.g., depth, flow, masks, confidence, 3D pose, overlay primitives), privacy-filtered, and/or otherwise transformed for improved downstream analysis or presentation. Originals and enhanced forms may be used together or separately.

Keypoint-Optional Operation; Optionality of Keypoints/Angles. Implementations may evaluate alignment using explicit keypoints/angles and/or without emitting an explicit keypoint list (e.g., vision-capable models using relational/part-centric features). Either approach (or their combinations) may be selected dynamically; model families/architectures are not limited by these examples.

Edge/Cloud Partition (481/482). The pipeline may be allocated between on-device (edge) (481) and cloud/server (482) based on device capability, bandwidth, and policy; a local-only variant omits the cloud. Latency-sensitive text-to-speech (TTS)/overlays may run locally while model-heavy analysis executes remotely.

Privacy & Data Lifecycle Controls. Anonymization/Redaction (485). On-device masking/cropping, metadata minimization, user-region crops, and session-scoped identifiers prior to any egress.

Compression/Cache (486). Policy-controlled compression and caching on device and/or server with bounded retention.

Encrypted Transport (500). TLS/HTTPS (Transport Layer Security/Hypertext Transfer Protocol Secure) or equivalent for any networked exchange; integrity protections.

Retention/Delete Controls (487) & Pseudonymous Session IDs (488). Explicit retention windows and pseudonymous association.

Graceful Degradation/Offline. Under degraded connectivity, the client prioritizes cached brief segments/overlays and defers bandwidth-heavy content; a local-only mode may run the full pipeline on device.

Network Topologies; Intermediary Server (165). Requests may flow directly or via an intermediary server (165) that, for example, inserts short-lived credentials, normalizes requests, applies policy gates, and/or performs limited caching; privacy/transport controls apply on each hop.

Coach Co-Pilot and Provenance. Proposed segments (471-473) may be reviewed/reshaped by an automated or human coach before presentation, with provenance tags (“AI”, “Coach”, “AI+Coach”) persisting to the user. Safety and timing policies apply; safety-critical cues may bypass gating.

AI agent (150). As used herein, an “AI agent” is any software, firmware, hardware, service, and/or orchestration of sub-components that (alone or in cooperation) performs algorithmic inference and/or procedural logic over one or more inputs to produce analyses, guidance, and/or artifacts for presentation to a user. Inputs can include pixels (e.g., images, clips, video), audio, wearable or other sensor telemetry, and/or references or templates (e.g., items from a model/pose store), together with metadata or instructions. Outputs can include, without limitation, (i) text or audio guidance, (ii) pixels (e.g., images, clips, video), (iii) overlay directives and other renderable geometry, and/or (iv) multi-segment responses suitable for client-side timing and selection. An AI agent may be monolithic or modular; deterministic, stochastic, or hybrid; rule-based, ML-based, or both; and may be implemented locally on a client device, remotely in a server or cloud, across an edge/cloud partition, and/or via an intermediary service. It may incorporate multiple cooperating “sub-agents” (e.g., pose, movement, breathing, error classification, feedback generation, response segmentation), sequence or ensemble models, and/or tool calls (e.g., vision encoders, transformers, speech/NLP (natural language processing), or rule engines). It may also retrieve or consume stored references or parametric templates, request or trigger additional capture, and/or attach provenance to generated content. Architectures, training data, model families, frameworks, and deployment topologies are implementation choices and are not limiting.

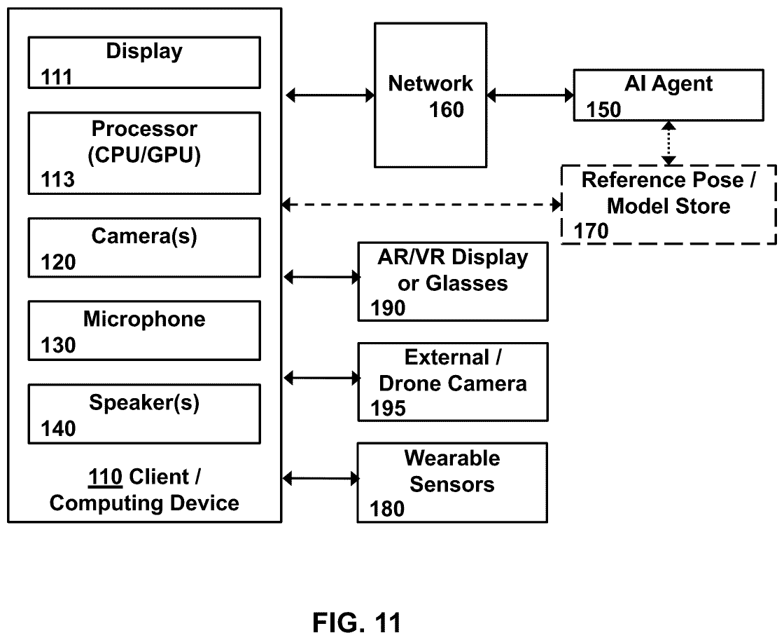

### System Architecture (FIG. 11)

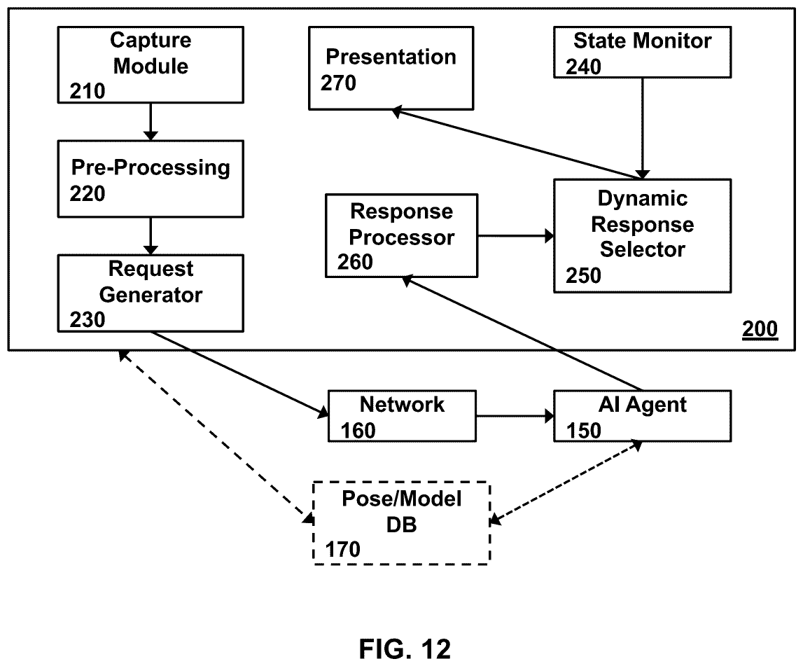

FIG. 11 depicts a representative deployment according to some embodiments of the technology disclosed in this patent document in which a client/computing device (110) acquires visual and audio inputs via camera(s) (120; RGB 121; depth 122; IR 123; LiDAR 124; ToF 125; thermal 126) and/or microphone (130), executes a client application (200) on a processor (CPU/GPU) (113), renders feedback on display (111) and/or speaker(s) (140) or in an AR/VR display or glasses (190), and communicates over a network (160) with an AI agent (150). The model/pose store (170) may reside on the client device and/or on a server. In client-hosted embodiments, the client retrieves reference media from 170 and may transmit it with user media to the AI agent for analysis. In server-hosted embodiments, the AI agent retrieves reference media from 170. These two options for the model/pose store (170) are indicated in FIGS. 11 and 12 using dashed lines for the element 170 and its connections. Optional wearable sensors (180) and an external camera(s) (e.g., a drone camera(s)) (195) augment fidelity and coverage.

Capture and on-device handling. Visual and audio signals are ingested locally into the device processor (113). This enables pre-processing (220) (e.g., resolution/aspect normalization, user-region cropping, background suppression) and privacy steps (e.g., anonymization/redaction (485)) prior to egress, and supports low-latency presentation paths (text-to-speech and overlays) on 111/140/190. When available, multiple cameras (121-126) and wearables (180) contribute to fused features that stabilize analysis under difficult conditions.

Networked analysis and responses. The client establishes a secure link (encrypted transport (500)) over 160 to the AI agent (150). Requests may contain user media and context; in some embodiments they also include the selected reference image or video (when, e.g., 170 is local to 110), allowing the agent to compare the user directly to that reference. The agent returns a multi-segment response (e.g., brief commands, detailed instruction, additional info) that the client's DRS (250/474) selects for immediate presentation vs. deferred review.

Model/Pose store placement. The model/pose store (170) can be local on the client (e.g., user-curated references and templates) and/or remote near the AI agent; stores may be synchronized or federated. If 170 is local, the request generator (230) may transmit both user media and a selected reference from 170 to the agent; if 170 is remote, the agent retrieves the needed reference.

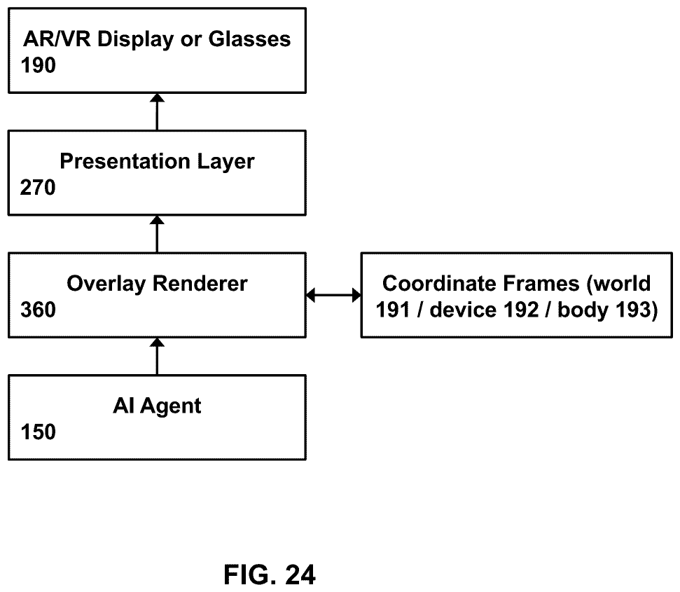

Presentation paths. Visual guidance (e.g., overlays (360)) and audio cues are rendered by the client in 111 or 190. Overlays are produced using directives returned by the agent or derived locally, and, in AR/HMD embodiments, may be placed in world/device/body frames (see FIG. 24).

External and wearable sources. An external/drone camera (195) can stream to the device for wider or adjustable viewpoints; wearables (180) (e.g., IMU, respiration) provide telemetry that the client can forward or fuse to improve confidence and breathing synchronization analysis.

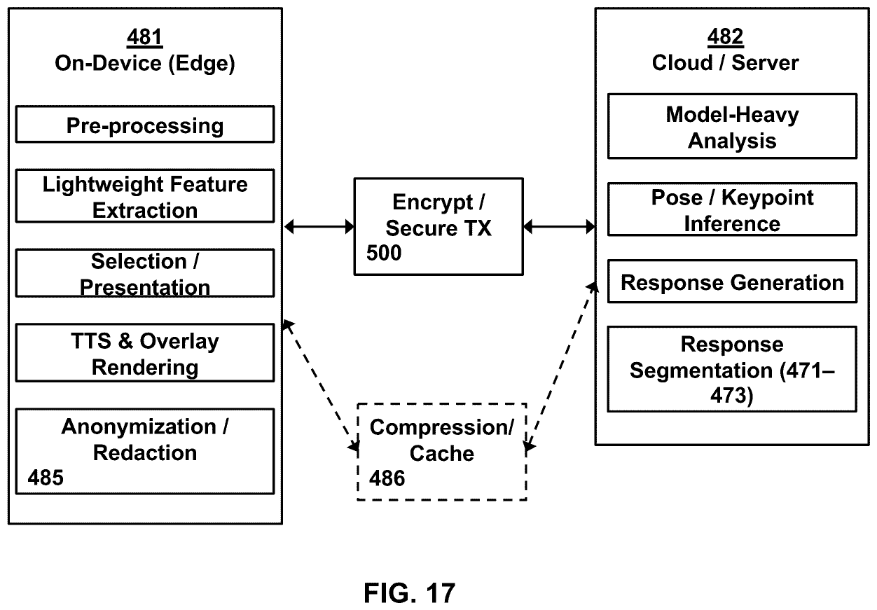

Security, partitioning, and caching. The system supports edge/cloud partitioning (e.g., on-device redaction/text to speech (TTS)/overlays; cloud-scale modeling and response bundling) and privacy controls including redaction (485), encryption (500), and caching (486), as further detailed in FIGS. 17-18 and 23.

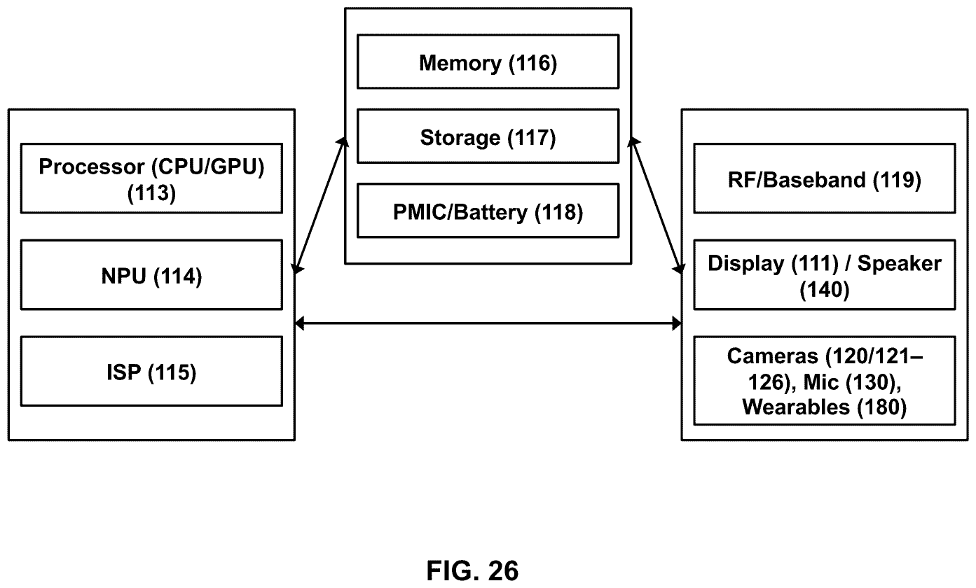

### Handset Hardware Block Diagram (FIG. 26)

FIG. 26 illustrates a representative mobile handset platform according to some embodiments which is suitable for the client device (110). A system-on-chip integrates a processor (CPU/GPU) (113), neural processing unit (NPU) (114), and image signal processor (ISP) (115), coupled to memory (116), non-volatile storage (117), power management IC/battery (118), and RF/baseband (119). Peripherals include display (111), speaker (140), microphone (130), camera(s) (120/121-126), and wearables (180) connected via short-range radios; an external/drone camera (195) can link wirelessly through the RF stack.

Capture and imaging pipeline. Sensor outputs from 120/121-126 enter the ISP (115) for demosaic, noise reduction, HDR, depth alignment, and other conditioning before being written to memory (116). Frames and metadata are then available to the client app executing on 113, and to on-device models running on 114 (when enabled by partition policy). This path supports multi-sensor fusion (e.g., RGB+depth/IR/ToF/LiDAR/thermal) for robust pose estimation in hard conditions.

On-device AI and graphics. The NPU (114) accelerates lightweight vision/audio models (e.g., user-region cropping, voice activity detection (VAD), coarse keypoints), while GPU/CPU (113) executes the client application (200), overlay rendering (360), and text-to-speech, minimizing round-trips for time-critical presentation. Model weights and feature caches reside in 116/117 as allowed by policy.

Audio and I/O. The microphone (130) routes to audio front-end blocks on the SoC; decoded or synthesized audio is played via speaker (140). The display (111) presents UI, side-by-side comparisons, and overlays; in AR/HMD use, the device streams overlay directives to 190 via the client presentation layer.

Network and peripherals. RF/baseband (119) provides cellular/Wi-Fi/BLE connectivity for secure communication to the AI agent (150) (via 160) and for pairing to wearables (180); in some embodiments, an external/drone camera (195) streams through the same RF path to the device.

Power/thermal and partitioning signals. The power management integrated circuit (PMIC)/battery (118) exposes thermal and power status that can steer the edge/cloud partition policy (e.g., throttle local inference or shift more work to cloud (482)), consistent with the system-level partition and security flows (redaction (485), encryption (500), caching (486)).

### Client Application and Dynamic Response Selector (DRS) (FIGS. 12, 14, 20)

In some embodiments of the disclosed technology, as shown in FIG. 12, a client application (200) executes on a client device (110) and orchestrates capture, request formation, reception and processing of multi-segment responses, and context-aware presentation. As shown in FIG. 12, the application comprises a capture module (210), pre-processing (220), a request generator (230), a response processor (260), a presentation layer (270), a state monitor (240), and a Dynamic Response Selector (DRS) (250); the app communicates with an AI agent (150) over a network (160) and may access a model/pose store (170). When 170 is local, 230 may include the selected reference image/video from 170 in the request.

Capture and pre-processing. The capture module (210) acquires media from the device's cameras and microphone (e.g., RGB, depth, IR, LiDAR, ToF, thermal; and audio), either continuously or on demand (e.g., voice/gesture trigger). The pre-processing module (220) may normalize resolution and aspect ratio, perform background suppression or user-region cropping, compute capture-quality flags (e.g., low light/backlight/stability) and, in some embodiments, extract lightweight features such as coarse keypoints or audio VAD/keyword cues to include as context. Privacy measures such as face blurring/cropping may also execute prior to transmission, consistent with the security and edge/cloud partitioning.

Request formation and store access. The request generator (230) packages the captured media with context (e.g., corrections-count, mode, app state) and forms a request to the AI agent (150) via network (160). When the model/pose store (170) is local to the client, the request can include both (i) user media and (ii) a selected reference image or video retrieved from 170; when 170 is remote, the agent may retrieve the reference itself. In either case, he agent can evaluate the user against reference media or parametric reference models/templates sourced from 170. Transport occurs over encrypted channels with optional on-device redaction/compression prior to egress.

Response processing and presentation. The AI agent (150) returns a multi-segment bundle comprising, for example, brief commands (471), detailed instructions (472), and additional information (473). The response processor (260) unpacks and registers segments (471/472/473) and overlay directives for the DRS (250). The DRS (250), using selection logic (474) and inputs from 240 and phase-aware features (610-615/620), commands the presentation layer (270) to render the chosen segment(s).

State monitoring and DRS. The state monitor (240) samples application state and salient user actions (e.g., “practicing,” “hold/review,” “idle/explore”) and exposes those to the DRS (250). The DRS applies selection logic (474) (e.g., rule-based, ML-based, or hybrid) to choose which segment(s) from the bundle to surface now and which to defer, balancing immediacy, comprehension, and resource constraints (e.g., bandwidth/battery). The resulting selection is rendered by 270 in the appropriate modalities (text, audio, overlay, video).

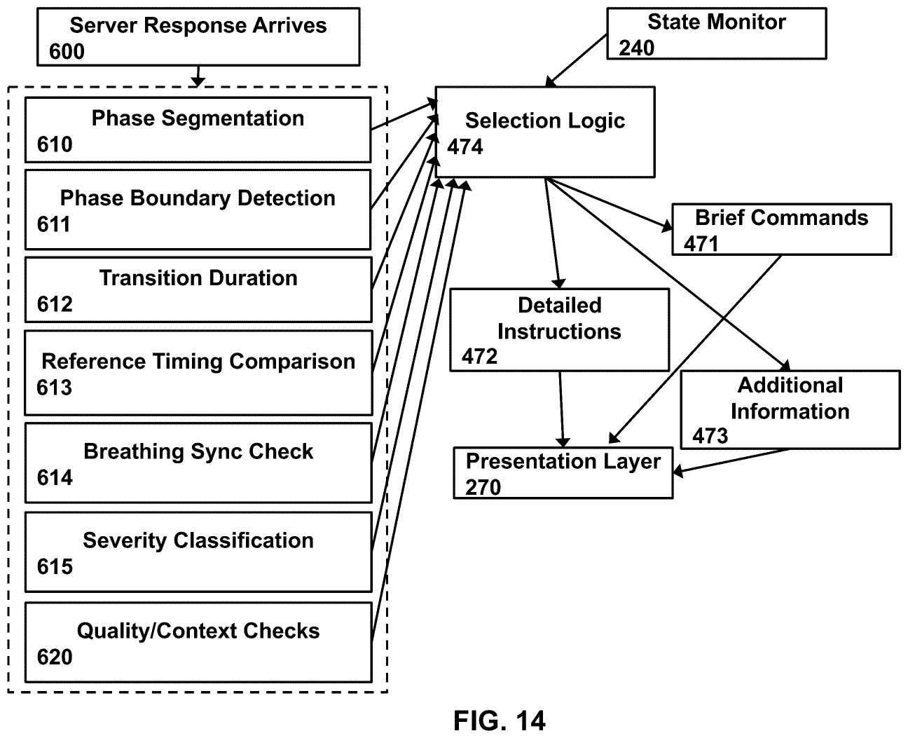

FIG. 14 shows DRS feature acquisition and selection according to certain embodiments of the disclosed technology. When a multi-segment response arrives (600), the client obtains phase-aware features (610-615) and quality/context signals (620) that guide presentation timing and content. Quality/context (620) includes capture-condition and runtime signals (e.g., lighting/backlight/exposure, framing/field of view (FOV)/distance, stability, bandwidth/battery/confidence) used by the DRS. When the AI agent includes these features in its reply, the client uses them directly; otherwise, the client derives equivalent signals locally from the returned labels/overlays and its recent capture context (e.g., timestamps, short frame buffer, wearable ticks), without re-running cloud-grade perception models. The features comprise phase segmentation (610), phase-boundary detection (611), transition duration (612), reference-timing comparison (613), breathing-synchronization checks (614), and severity classification (615), together with quality/context checks (620). The selection logic (474) of the DRS (250) consumes these inputs with app state (240) to choose one or more segments (brief commands (471) for imminent transitions or safety-critical items, detailed instructions (472) for holds/review, and additional information (473) for exploration) which the presentation layer (270) renders.

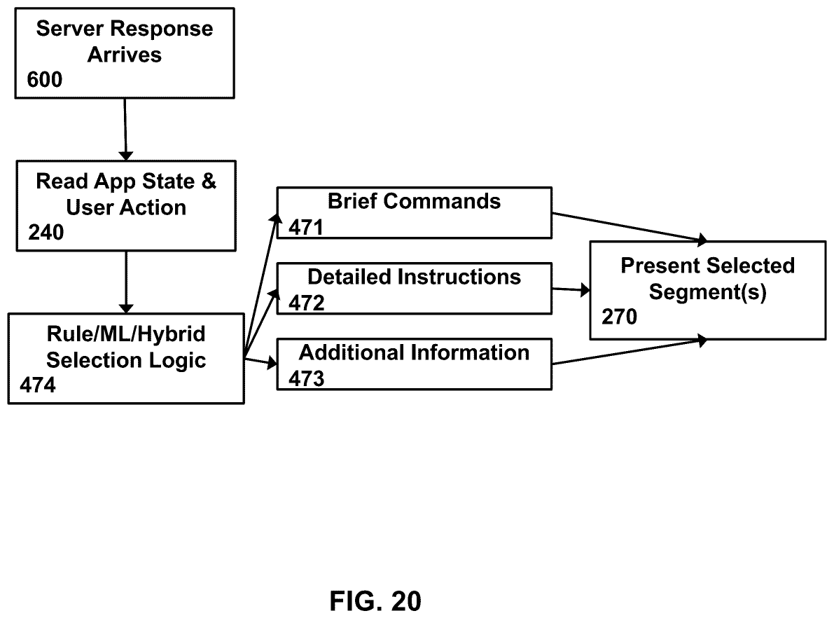

FIG. 20 shows trigger and state sampling according to some embodiments of the disclosed technology. When a multi-segment response is received (600), the application consults the state monitor (240) to determine the current and recent app states and user actions (e.g., practicing with an upcoming predicted (by, e.g., the AI agent) phase boundary; holding a pose; paging through information). The state monitor may also expose runtime constraints (bandwidth/battery) and confidence/quality signals.

Selection logic and outputs. The selection logic (474) uses the sampled state together with phase-aware features (see FIG. 14) to select brief (471), detailed (472), additional (473), or combinations thereof; the presentation layer (270) then renders the selection (e.g., immediately), with any non-selected segments deferred for review. For example, during a time-critical transition under low bandwidth, the DRS may emit TTS brief commands immediately and defer detailed instruction until the next hold.

Behavioral examples. In a Vinyasa transition with an upcoming boundary in ˜300 ms, the DRS surfaces a single brief cue (“step right foot forward”) for immediate playback and schedules breathing resynchronization guidance for the next hold. During post-session review, the DRS elevates detailed instruction and side-by-side visuals; in idle/explore state, supplemental tips may be presented.

The flow shown in FIG. 20 can be executed per response and may preempt or coalesce outputs to minimize distraction during high-motion phases while maximizing comprehension during pauses.

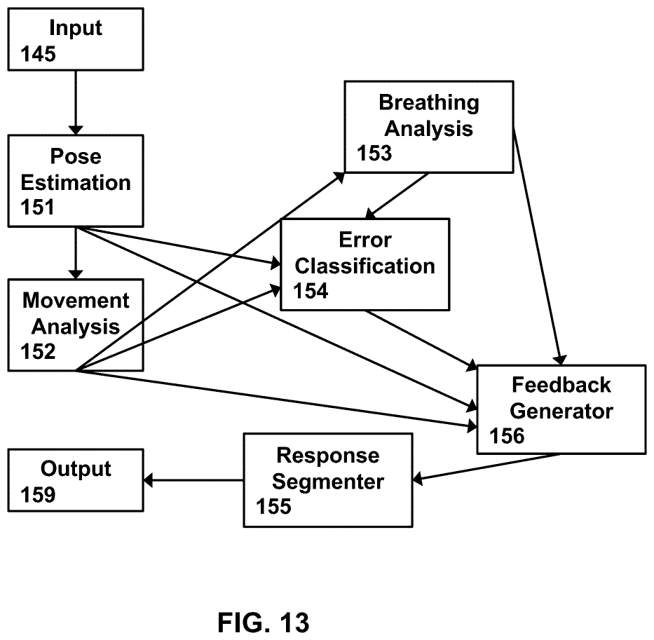

### AI Agent Pipeline and Pose/Movement/Breathing Analysis (FIG. 13)

As shown in FIG. 13, in some embodiments of the disclosed technology, the AI agent (150) receives inputs (145) that can include an image, short clip, or video (and, in some embodiments, audio). The agent consults references and/or templates from the model/pose store (170) and executes a pipeline comprising pose estimation (151), movement analysis (152), breathing analysis (153), error classification (154), a feedback generator (156), and a response segmenter (155) that emits the multi-segment output (159). The architecture accommodates both (i) implementations that use explicit keypoints and angles and (ii) keypoint-optional implementations (e.g., vision-capable LLM/VLM or other models) that operate on implicit visual representations and return natural-language corrections and/or overlay parameters without exposing a keypoint list to the client.

Pose estimation (151): explicit or implicit representations. Explicit option: The agent may predict 2D/3D keypoints and confidences and/or joint angles; in short clips, these signals can be temporally lifted (i.e., aggregated across neighboring frames to infer a consistent 3D pose and/or to stabilize trajectories) before downstream analysis. Implicit option (keypoint-optional): In keypoint-optional embodiments, a vision-capable model evaluates alignment and composes feedback without emitting a keypoint list to the client. A vision-capable model (e.g., VLM/LLM or other transformer-based vision model) encodes the image into part-centric tokens and attention maps that capture limb orientation, symmetry, and relative distances without materializing joint coordinates. Both approaches yield a pose representation suitable for comparison to references (images/videos or parametric templates) from 170.

How implicit alignment works, according to some embodiments: The model (i) localizes salient body regions (arms, legs, pelvis, spine) via attention; (ii) computes relative geometry (e.g., “knee stacks over ankle,” “torso vertical vs. floor,” “arms collinear at shoulder height”) as text-grounded constraints; (iii) measures directional discrepancies (“rotate pelvis toward side,” “widen stance”) against a target described by a reference image/video or textual target; and (iv) produces natural-language corrections and/or overlay directives (lines, arrows, markers), without returning a skeletal keypoint list to the client.

Movement analysis (152): temporal reasoning and phase/timing. From a sequence of frames, the agent segments motion into phases (ingress/hold/egress), estimates phase boundaries and transition durations, and compares cadence/timing against reference timing retrieved from 170 (or generic templates). This can be implemented with sequence models (e.g., transformers) operating over explicit pose trajectories or over implicit visual tokens; in both cases the outputs include phase labels/timestamps and timing deltas used downstream. Sequence models (e.g., transformers) can operate either on explicit pose trajectories (e.g., time-series of keypoints/angles) or on implicit visual tokens produced by a vision model, enabling phase/timing analysis and deviation scoring without obligating the agent to output a keypoint list.

Breathing analysis (153): visual, audio, and fused cues. Breathing is inferred from one or more modalities: chest/abdomen expansion in video, audio (breath noise, cadence), and/or wearables (e.g., respiration band, PPG-derived rhythm). The agent aligns the estimated inhale/exhale phases to movement phases to determine breathing synchronization (e.g., “inhale on Upward Dog,” “exhale on Downward Dog”).

Error classification (154): converting deviations to severity labels. Spatial and temporal features are aggregated to classify deviations (e.g., minor/moderate/severe). With explicit keypoints, this can involve joint-angle thresholds and signed offsets; with implicit representations, the model maps text-grounded relational discrepancies (e.g., “front knee ahead of ankle,” “back heel lifted”) to severity tiers. Severity and breath-sync flags prioritize safety-critical cues.

Feedback generator (156): natural language+overlays. Given pose/movement/breathing signals and classification results, the agent composes text explanations and corrective strategies, and, where applicable, attaches overlay directives (360) (lines, arcs, arrows, markers) that visualize targets and corrective directions on the client. This stage also supports pose identification (naming the asana) and personalization.

Response segmenter (155) and outputs (159): bundles for the DRS. The generator's content is packaged into a multi-segment bundle (e.g., brief commands (471) for in-motion cues, detailed instructions (472) for learning/review, and additional information (473)) and returned as outputs (159). The client's DRS later selects which segment(s) to present based on context.

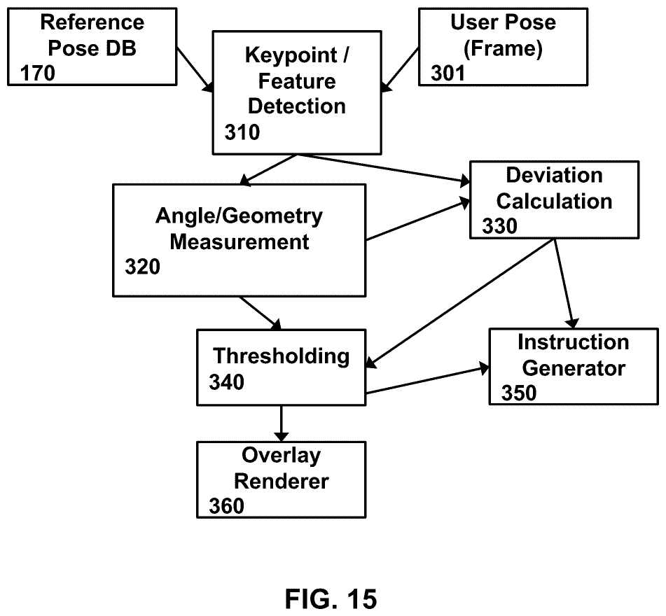

### Pose Comparison, Overlays, and Objective Guides (FIG. 15)

According to certain embodiments of the disclosed technology, as shown in FIG. 15, a user frame (301) is evaluated relative to one or more reference poses or movement sequences drawn from the model/pose store (170). When 170 is local, the client may include the selected reference with the request; when 170 is remote, the AI agent retrieves it. Keypoint/feature detection (310) enables angle and relational measurement (320); deviation computation (330) compares those measurements or equivalent relational features to reference constraints, and thresholding (340) maps magnitudes to qualitative severity levels used to prioritize overlays and cues. Instruction generation (350) transforms deviations into concise, positively framed cues, while an overlay renderer (360) projects objective guides (lines, arcs, markers) derived from the same geometric primitives used for comparison (e.g., ankle vertical, shoulder band, joint-angle arcs) and associated with user-curated references in 170 on the client display or AR/HMD.

Inputs and normalization (170, 301→310). In the embodiment illustrated in FIG. 15, the user frame (301) is a single image or a key frame from a short clip; the reference(s) (170) can be curated by the user (“Add-a-Pose”) or system-provided and may also encode target constraints (e.g., “front knee stacked over ankle,” “pelvis neutral”). Prior to metric extraction, the frame(s) can be cropped to the user region and normalized for scale/aspect (see pre-processing (220) and objective overlays associated with user-curated references in 170). These steps can improve stability of downstream angle and alignment checks.

Keypoint/feature detection (310). In keypoint-based embodiments, the agent predicts 2D/3D joint locations with confidences and (optionally) limb vectors or segmentation masks. In keypoint-optional embodiments (e.g., a vision-capable LLM/transformer), the model extracts part-centric features and relational cues (e.g., limb orientation, symmetry, relative distances) without emitting an explicit keypoint list; these internal features are treated as functional equivalents for the computation stages that follow. Either representation feeds 320 and 330.

Angle and geometric measurement (320). From the detected joints or part-centric features, the pipeline measures signed joint angles (e.g., elbow, knee, hip), segment orientations (e.g., forearm vs. horizontal), and alignment relationships (e.g., “knee vertical above ankle,” “torso near vertical,” “arms collinear at shoulder height”). Measurements can be made scale-and rotation-aware (e.g., using vertical/horizon estimates or camera metadata) so comparisons remain meaningful across camera placements. The 320→340 path emphasizes overlay-ready geometry: arcs at joints, straight alignment guides, and target lines the renderer can draw.

Deviation computation (330). Measured geometry is compared to reference constraints from 170. The module computes signed deviations and directions of correction (e.g., “rotate pelvis +10-15° toward neutral,” “press back heel down,” “widen stance 3-5 cm”), weighting by detector confidence. Where 170 supplies parametric templates (ranges/targets), deltas are computed directly; where 170 supplies reference media, deltas are derived by relational comparison to the reference (e.g., user-knee line vs. ankle line) using the same geometry.

Thresholding and labeling (340). Deviations are mapped to qualitative severity levels (e.g., minor, moderate, critical) using configurable thresholds (global or pose-specific). Low-confidence signals are suppressed; safety-critical patterns (e.g., knee far ahead of ankle, excessive lumbar extension) are up-tiered regardless of magnitude. The 340 labels are also used to prioritize overlay elements (e.g., show only high-value guides during practice) and to color/weight overlays where applicable.

Instruction generation (350). The 350 module transforms deviations into concise, positively framed cues and more detailed explanations. Each cue can carry (i) the target (what to change), (ii) the direction/amount (how to change), and (iii) optional rationale (why it matters). Examples: “Align knee over ankle,” “Press back heel down; lengthen the back leg,” “Rotate pelvis toward neutral. ” These instructions can be emitted as text and/or parameters for TTS; they later populate brief vs. detailed segments in the bundle (see FIG. 13-14; FIG. 15 focuses on the local conversion from deviations to cues, independent of bundling).

Overlay renderer and objective guides (360). The overlay renderer (360) projects objective elements (e.g., lines, arcs, angles, markers, arrows) anchored to measured or inferred control points. Typical guides include, for example: a vertical “stack” line through the ankle to show knee alignment; a shoulder-height band to cue arm level; angle arcs at elbow/knee/hip; directional arrows indicating rotation or translation. Overlays are produced for on-device display (111) and, in AR/HMD embodiments, in world/device/body coordinate frames (see FIG. 24).

Keypoint-optional operation within the embodiment shown in FIG. 15. When the agent operates without returning explicit keypoints, 310 furnishes implicit relational features sufficient to: (i) derive overlay anchors for 360 (e.g., approximate joint centers from part masks or attention peaks); and (ii) compute deviations (330) against 170 using relative predicates (e.g., “ankle below knee,” “arms horizontal,” “torso near vertical”).

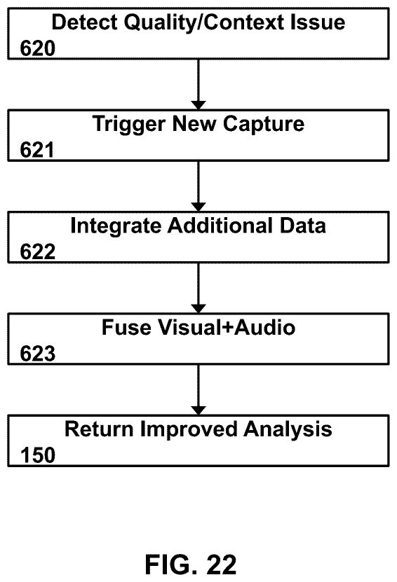

Interaction with quality/context and presentation. While FIG. 15 defines the geometry-to-guidance pipeline, quality/context (620) from capture (lighting, framing, stability) can steer which overlays to show or whether to surface camera/lighting tips instead of nuanced form cues (see FIGS. 14, 22). Generated overlays and cues are ultimately selected and timed by the DRS (250/474) (see FIGS. 12, 14, 20), which may, e.g., favor minimal overlays and a single brief command during high-motion moments and defer detailed overlays for review.

An example (FIG. 15 semantics in practice). Pose: Warrior II. Targets (170): knee stacked over ankle; arms at shoulder height; pelvis neutral; back heel grounded. 310: detects joints or infers arm/leg/torso parts. 320: measures knee-ankle vertical alignment; shoulder-line height; pelvis rotation; heel contact proxy. 330: computes signed deltas (knee forward +8°, arms −6° below level, pelvis +12° rotated). 340: labels knee and pelvis as moderate/critical, arms as minor. 350: cues: “Align knee over ankle,” “Rotate pelvis toward neutral,” “Raise arms to shoulder height.” 360: draws a vertical stack line through the ankle with an arrow from the knee toward the line; a horizontal guide at shoulder height; and a pelvis rotation arc with arrow showing direction.

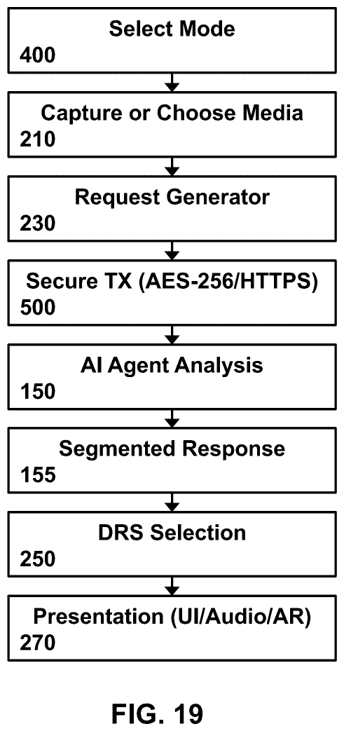

### End-to-End Method Flow (FIG. 19)

FIG. 19 illustrates a representative session that runs from mode selection (400) through capture/choose media (210), request generation (230), secure transport (500) to the AI agent (150), receipt of a segmented response (155), client-side selection (DRS 250), and presentation (270) on device 111 or AR/HMD 190. The flow is agnostic to model architecture (keypoint-based or keypoint-optional) and to store placement for 170 (local/federated vs. server-side).

Mode selection (400)→capture or choose media (210). At session start, the user picks a mode such as, e.g., real-time capture, gallery comparison, or reference exploration. In real-time, 210 acquires frames/audio from 120/121-126 and 130; in gallery, 210 reads an image/clip via the picker; in explore, 210 may fetch only a reference from 170 for side-by-side or tutorial review. Voice/gesture controls can initiate capture hands-free.

Optional pre-flight/quality checks. Before or during capture, the app may evaluate quality/context (620) (framing, distance/FOV, lighting/backlight, stability) and prompt adjustments (e.g., “step back,” “increase light”) or switch cameras, improving downstream fidelity.

Request generation (230). The request generator (230) packages media with context (app state, timestamps, device class, corrections-count, requested segment types, etc.). If 170 is local, 230 may include the selected reference (image or clip) in the request; if 170 is remote, the agent will acquire it (see FIG. 11-12, 15). Optional on-device pre-processing can crop to the user region, redact faces (485), and compress/cache (486) prior to egress.

Secure transport (500). The client transmits over an encrypted channel (TLS/HTTPS (Transport Layer Security/HyperText Transfer Protocol Secure), session/rotation policy) and applies the privacy/data-lifecycle steps described for FIGS. 17-18 and FIG. 23 (redaction 485, compression/caching 486). Network topologies may include direct 110→150 or via an intermediary server for API-key handling and normalization.

AI agent analysis (150). The AI agent (150) executes the pipeline of FIG. 13: it ingests 145, performs pose estimation (151) (explicit keypoints/angles or keypoint-optional visual reasoning), movement analysis (152), breathing analysis (153), and error classification (154), then composes feedback with a feedback generator (156). The agent can compare user media to reference media/parametric templates from 170, including users' curated “Add-a-Pose” references.

Segmented response (155). The agent packages the output into a multi-segment bundle (155) (for example, brief commands (471), detailed instructions (472), and additional information (473)) and may attach overlay directives (360) using a schema for bundles and/or overlays. Bundling reduces round-trips and enables client-side timing/selection.

DRS selection (250). Upon response arrival, the client's Dynamic Response Selector (250) applies selection logic (474) (rule-based/ML/hybrid per FIG. 20) using (i) application state/user actions (240); (ii) phase-aware and context signals (610-615, 620) obtained from the reply or derived locally; and (iii) runtime constraints (bandwidth/battery). During high-motion phases, the DRS favors brief suggestions; during holds/review, it can surface detailed instructions with overlays; when quality is inadequate, it may surface camera/lighting tips rather than nuanced form cues. (See FIGS. 14 and 20 for inputs and decision flow.)

Presentation (270). Selected segments are rendered by 270 as text/audio and overlays on display (111) or AR/HMD (190). Overlays can use objective guides (lines, arcs, markers) and a directive schema; audio may be TTS or server-provided clips. The client can cache selected segments for post-session review and operate gracefully under degraded connectivity.

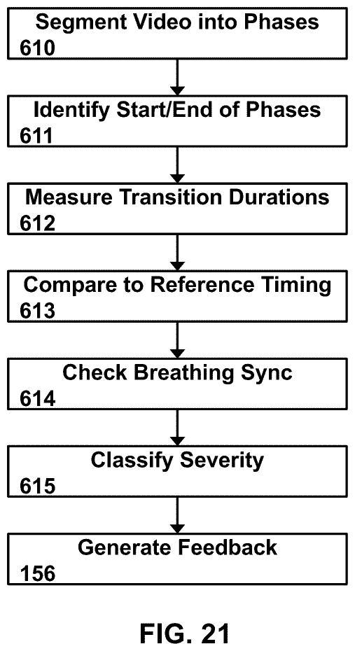

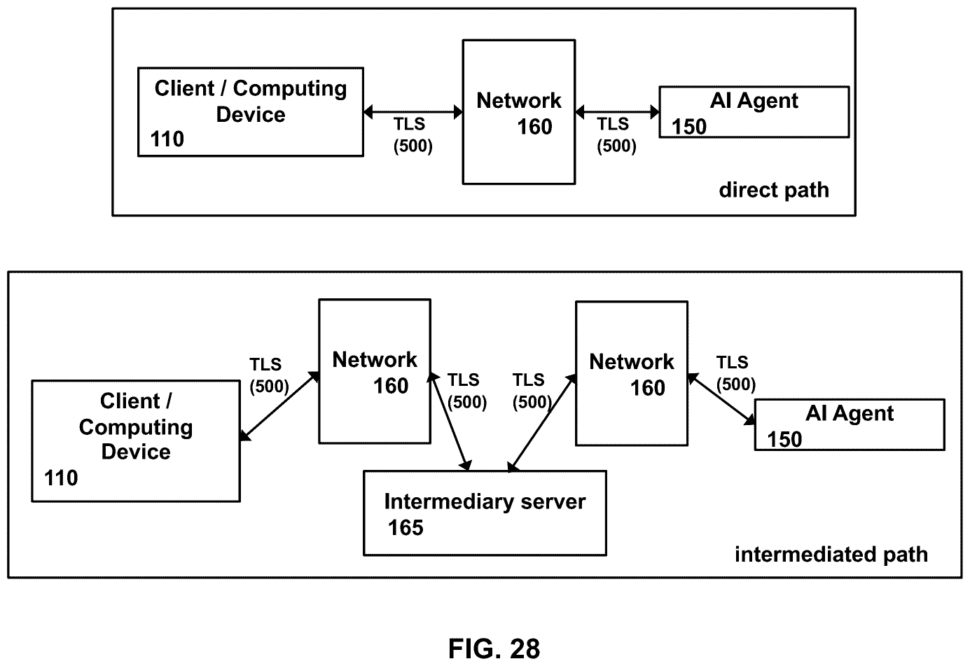

Variants and loop-backs (FIG. 19). In some embodiments, the FIG. 19 method exercises additional branches: (i) Quality/fusion loop: when quality/context (620) flags occlusion, poor lighting, or insufficient coverage, the client triggers additional capture (621), integrates the new data (622), fuses modalities (623), and resubmits for improved analysis; see FIG. 22. (ii) Edge/cloud partition: latency-sensitive steps (e.g., TTS, overlays, optional redaction) may run on-device (481) while model-heavy analysis executes in the cloud (482) with encryption (500) and caching (486); see FIGS. 17, 18 and 23. (iii) Reference sourcing: if the model/pose store (170) is client-hosted, the request may include the selected reference; otherwise the AI agent (150) retrieves it (see FIG. 11-12, 15). (iv) Static vs. sequence: for single images, the DRS primarily uses severity (615) and quality (620); for sequences, it additionally uses 610-614 (phases, boundaries, durations, reference-timing, breathing sync) as described in FIGS. 14 and 21. (v) Network topology: in deployments with an intermediary server, the secure request (500) may traverse the intermediary for credential handling and normalization before reaching 150; see FIG. 28.

Data carried in the FIG. 19 exchanges (illustrative). Requests may include requestedSegments, corrections-count, device metadata, timestamps, and (when local) a reference image/video; responses carry pose identification, corrections (detailed), brief commands, tips, and optional products/links, organized as segments for the DRS.

### Movement Sequence Analysis (FIG. 21)

FIG. 21 details, according to some embodiments of the technology disclosed in this patent document, analysis of a video or frame sequence to produce time-aligned, phase-aware signals (610-615) that guide feedback generation (156) and, when included in the agent's reply, the client's DRS selection logic (474) for timing and content selection. From the incoming clip (optionally with audio and/or wearable telemetry) the system: segments motion into phases (610), detects boundaries (611), measures transition durations (612), compares timing to a reference (613), assesses breathing synchronization (614), and classifies deviations by severity (615). The pipeline supports explicit keypoints/angles or implicit visual features/tokens, and does not require emitting a keypoint list to the client (in other embodiments, the keypoint list can be emitted to the client).

Inputs and references. The analysis consumes a timestamped clip (or sliding window of frames), optional audio (for breathing), optional wearables (respiration/cadence proxies), and reference material from the model/pose store (170). References may be media (reference video/frames) and/or parametric templates (phase names/order, target phase durations, cadence envelopes, and expected breath-phase relationships). When 170 is local, the client may include the selected reference in the request; when 170 is remote, the agent retrieves it.

(610) Phase segmentation. The sequence is partitioned into named phases (e.g., ingress→hold→egress or domain-appropriate equivalents). Implementations may operate on (a) explicit pose trajectories (time-series of joint coordinates/angles) or (b) implicit visual tokens (part-centric embeddings/attention features) that capture body regions and their relations without exposing coordinates. Output: a timeline of phase labels with start/end timestamps and confidences.

(611) Phase-boundary detection. Boundaries between phases are detected and assigned precise timestamps; the module also estimates or predicts time-to-boundary for imminent (or upcoming) transitions, which the DRS uses to prioritize brief commands just before a change. Boundary confidence reflects signal quality, motion salience, and (when available) agreement across modalities.

(612) Transition duration. For each transition, the system measures the observed duration from time-stamped boundaries and computes a de-jittered duration estimate (and optional smoothness score) that is robust to variable frame rates and dropped frames. These durations provide cadence cues (e.g., “slow/smooth the transition”), feed timing comparison (613), and can gate whether detailed instruction is deferred to a later hold.

(613) Reference-timing comparison. The observed phase timeline is aligned to reference timing drawn from 170 (e.g., a canonical template for the exercise or a user-selected reference clip) yielding ahead/behind offsets, per-phase duration deltas, and global cadence assessments. Results may be emitted as structured fields (e.g., {phase: hold, desired: 3.0 s, actual: 2.2 s, delta: −0.8 s}) and as summary flags (“hold too short,” “transition rushed”).

(614) Breathing synchronization. Breathing phase (inhale/exhale/hold) is estimated from one or more modalities (e.g., chest/abdomen motion in video, audio signatures, and/or wearables (respiration, cadence proxies)) and aligned to the movement phases, producing an in-sync/early/late assessment and, optionally, a suggested resynchronization point (e.g., “begin exhale at next egress”).

(615) Severity classification. Spatial/temporal deviations (including breath-sync results) are aggregated and mapped to severity tiers (minor/moderate/critical) according to pose-specific rules or learned thresholds. Safety-critical patterns (e.g., abrupt, high-load transitions or misalignments known to stress joints) are up-tiered. These labels help determine what feedback to surface first and what overlays to render.

Outputs and consumers. The module emits: (i) a phase-annotated timeline (610-613), (ii) a breath-sync assessment (614), and (iii) severity labels (615). These signals feed the feedback generator (156) which composes text and overlay directives and may be included in the reply so the client DRS (474) can time and select brief (471)/detailed (472)/additional (473) segments for presentation (270). If certain fields are omitted, the client may derive functionally equivalent signals from returned labels/overlays and local context (as described for FIG. 14).

Robustness, sensors, and quality/context. Quality and context (620) (lighting, framing/distance, occlusion, stability, bandwidth/battery) influence confidence and thresholds; when fidelity is insufficient, the system may trigger additional capture (621), integrate new data (622), and fuse modalities (623) (e.g., switch cameras or incorporate wearables), then re-analyze. Multi-camera arrangements and depth/IR/ToF/LiDAR support improve segmentation under difficult conditions.

Keypoint-optional embodiment. In keypoint-optional mode, a vision model produces implicit part-centric tokens and text-grounded relational predicates (e.g., “knee tracks over ankle,” “torso near vertical”) per frame. A sequence model operates directly on those tokens to produce 610-615 and the overlay/text directives used downstream without emitting a keypoint list to the client.

### Capture Triggers and Fusion (FIG. 22)

FIG. 22 shows a robustness loop that activates when quality/context (620) indicates low fidelity (e.g., poor lighting/backlight, occlusion, framing/distance/FOV issues, camera shake, low confidence, bandwidth/battery constraints) in certain embodiments of the disclosed technology. On detection, the client triggers additional capture (621), integrates the new data (622) into the current session context, fuses modalities (623) to improve signal quality, and re-analyzes via the AI agent (150).

(620) Quality/context detection. The client monitors capture and runtime conditions and flags issues such as: (i) lighting/exposure/backlight problems; (ii) framing/distance/FOV too tight/too wide; (iii) occlusion (self-occlusion or background clutter); (iv) instability/blur; (v) low confidence in pose/features; and (vi) bandwidth/battery constraints. These signals are the same quality/context (620) features that also inform the DRS (e.g., presenting camera/lighting tips when form cues would be unreliable).

(621) Trigger additional capture. On a 620 condition, the app can request more or different data, for example: Lighting/exposure: enable torch/flash (where safe), request a brighter scene, or adjust exposure/ISO. Framing/distance/FOV: prompt the user to step back, reposition, rotate to portrait/landscape, or move the device to a stable surface; optionally increase resolution or short-burst frame rate. Occlusion/coverage: switch cameras or viewpoints (front/rear or to an external/drone camera (195)), ask for a side angle or lift angle; in AR/HMD mode, suggest a different body orientation. Breathing analysis: capture a brief audio snippet or pull a recent wearable sample (respiration/cadence proxy). Depth/IR/ToF/LiDAR: request a short auxiliary burst if the device supports it to disambiguate limbs. These are “triggers”; the user action and/or device pipeline collects the requested increments.

(622) Integrate additional data. Newly captured data are time-aligned and normalized into the active session: Temporal alignment to existing clips via time stamps; deduplication of overlapping frames. Normalization (crop/user-region, resolution/aspect, color/depth registration) consistent with pre-processing. Association of wearable samples; sensor metadata recorded for fusion (camera intrinsics/extrinsics when multi-camera). Policy checks for redaction/compression/caching before re-submission (consistent with privacy/edge-cloud flow).

(623) Fuse modalities. The system fuses modalities to increase confidence and reduce ambiguity: RGB+depth/IR/ToF/LiDAR to stabilize limb separation and depth ordering; Multi-camera fusion using calibrated extrinsics (or lightweight homography approximations) to reconcile side/front views; Wearable+video rhythm alignment for breathing/tempo; Temporal smoothing across the expanded window to reduce jitter. Fusion outputs are the improved features/frames fed back through the analysis path.

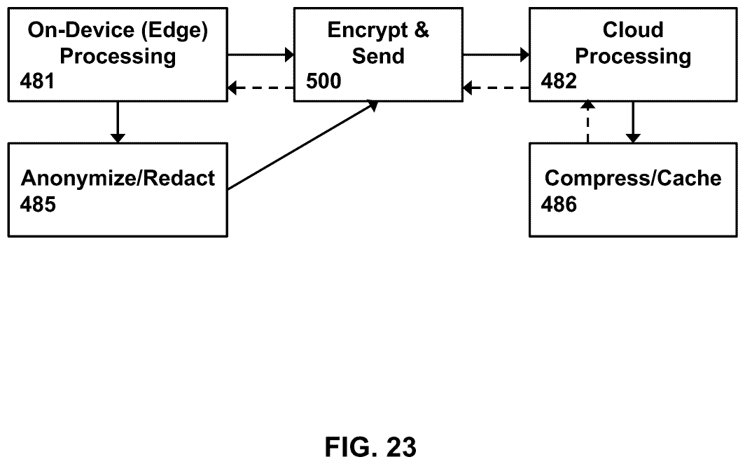

Re-analysis and outputs (→150). After 623, the client re-submits the augmented package to the AI agent (150) (secure transport as in FIG. 23). The agent runs the FIG. 13 pipeline on the improved inputs and returns an updated multi-segment bundle (with any overlay directives). The client's DRS (474) then uses the new results (along with 620) to time and select brief (471)/detailed (472)/additional (473) segments for presentation (270).

Immediate user guidance (620→DRS). In parallel with the 620→621→ . . . loop, the client may present camera/lighting hints immediately (e.g., “step back,” “increase light”), then apply the improved results when the re-analysis returns.

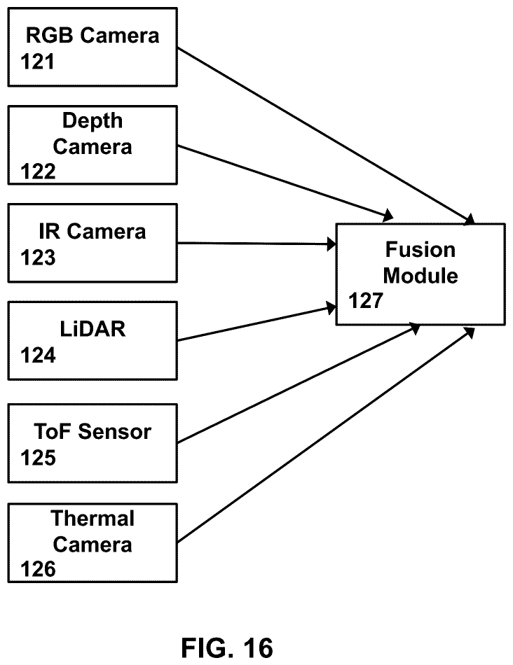

### Multi-sensor Capture and Fusion (FIG. 16)

FIG. 16 illustrates how, in some embodiments, the client device (110) improves robustness in hard conditions by using one or more camera modalities (e.g., RGB (121), depth (122), infrared (123), LiDAR (124), time-of-flight (125), and thermal (126)) and combining them in a fusion module (127). The fusion output strengthens downstream pose, movement, and breathing analysis (see FIGS. 13, 15, 21), reduces occlusion errors, and stabilizes geometry for overlays and AR presentation (see FIG. 24).

Roles of the modalities (121-126). RGB (121): primary visual signal for pose/appearance and overlay rendering. Depth (122)/ToF (125): per-pixel depth aids foreground segmentation, occlusion reasoning, 3D limb separation, and camera-to-subject distance estimation. Infrared (123): improves body/edge contrast in low-light or high-contrast scenes; useful for coarse silhouette and joint-adjacency cues when RGB is noisy. LiDAR (124): provides scene-scale geometry (planes, ranges) and supports large-space calibration (e.g., floor plane, camera-to-room scale) for more stable angle/height references and AR anchoring. Thermal (126): highlights warm regions and thoracic/abdominal thermal pulsation, useful for breathing rhythm in dim scenes and for skin-vs-background separation.

Fusion module (127). The fusion module (127) is a logical component that aligns and combines signals from one or more modalities (e.g., RGB (121), depth (122), IR (123), LiDAR (124), ToF (125), thermal (126)) into a unified representation with confidence weights for downstream use. It performs temporal synchronization (timestamp alignment), spatial registration (intrinsics/extrinsics, warps), quality-aware weighting (favoring higher-confidence sources), and emits fused features (e.g., person/limb masks, occlusion maps, aligned depth, floor plane). Implementations include: (i) software: a library inside the client app (200) using CPU/GPU kernels; (ii) hardware-accelerated: NPU/DSP kernels, or ISP-assisted registration paths on the device (110); (iii) firmware: microcode blocks on the sensor/SoC that pre-align depth/IR to RGB; and (iv) hybrid: early alignment on device with optional cloud-side refinement when policy allows. The module exposes fused outputs to both local consumers (e.g., overlay rendering, capture-quality checks, DRS context) and to the request path so the AI agent (150) can exploit multi-sensor cues under the same privacy/transport controls.

Synchronization and calibration in fusion (127). The fusion module (127) ingests streams from 121-126, aligns them temporally using timestamps, and spatially using intrinsics/extrinsics (or light-weight registration): Depth/ToF→RGB registration: map depth to RGB pixels to create aligned RGB-D frames; fill small holes; project to a common frame. IR/Thermal alignment: resample to RGB geometry (with lens model differences accounted for) so temperature/IR edges can inform the RGB silhouette. LiDAR scene cues: estimate floor plane/horizon, distances, and scale to normalize measurements and support AR/HMD frames (world/device/body; cf. FIG. 24). The output is a temporally consistent, co-registered set of per-pixel channels and/or fused features with confidences, ready for the app pipeline.

Fusion strategies. Fusion may be early, mid, or late: Early fusion (per-pixel): concatenate aligned channels (RGB, depth, IR/thermal) and derive masks (person/limb/occlusion) before any higher-level inference. Mid-level fusion (features): run light encoders per modality and fuse feature maps (e.g., attention-weighted by confidence) to stabilize joint/part inference and geometric primitives (e.g., vertical stack line, shoulder band) used later by the overlay renderer (360) (see FIG. 15). Late fusion (decision-level): combine parallel modality-specific estimates (e.g., RGB-only and RGB-D models) by confidence gating; fall back to RGB-only if auxiliaries are absent or low confidence. Fusion weights can be modulated by quality/context (620): for instance, emphasize IR when low-light is detected or emphasize depth when background clutter is high (see FIG. 22).

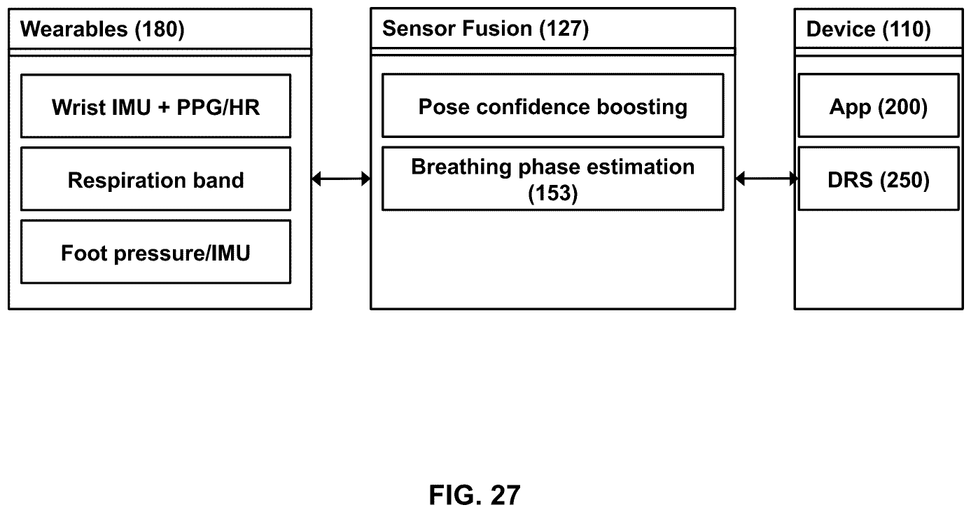

Breathing and movement support. Thermal (126) and depth/ToF (122/125) signals can amplify subtle chest/abdomen motion, assisting breathing analysis (153); IR (123) helps track torso/abdomen contours in dim scenes. These fused cues complement audio and wearables (see FIG. 27) when available and improve the (614) breath-sync signal in FIG. 21.

Multi-camera and external viewpoints. Fusion can incorporate multiple views (front/side) or an external/drone camera (195). When camera extrinsics are known/estimated, 127 reconciles viewpoints (triangulation or homography approximations) to reduce self-occlusion and improve phase and boundary detection for movement.

Degradation and policy. If certain sensors are unavailable or low confidence, fusion degrades gracefully to the available modalities. A device/thermal/bandwidth policy may disable high-cost sensors dynamically; the decision policy for edge/cloud (see FIG. 23) can also relocate heavier alignment to cloud when appropriate. In all cases, RGB-only operation remains supported.

Privacy, security, and retention. All modalities flow through the same privacy filtering/redaction (485) and encrypted transport (500) when data leaves the device; caching (486) and retention follow the same policy as for RGB, with modality-specific metadata redacted as needed (see FIG. 23).

Outputs and consumers. The fusion output includes aligned channels, occlusion/person masks, confidence maps, and optionally 3D support cues (floor plane, scale), which the app can: (i) pass along in the request (230) to the AI agent (150), and/or (ii) use locally to stabilize overlays and quality/context (620) assessment. Pose/movement/breathing analysis (151-153) benefits directly (cf. FIGS. 13, 21), and objective overlays (360) render more reliably (cf. FIG. 15).

### Wearables and Auxiliary Signals to Fusion (FIG. 27)

FIG. 27 shows representative wearables (180) (e.g., wrist IMU+PPG/HR, respiration band, foot-placement/pressure sensors or shoe IMUs) that communicate over BLE/Wi-Fi to the device (110) in some embodiments of the disclosed technology. Their telemetry is combined by the sensor fusion module (127) with recent visual/audio context to boost confidence, stabilize phase/timing, and improve breathing estimation (153). The fused signals are then consumed by the client application (200) and its DRS (250) to time and prioritize brief/detailed guidance.

Wearable inputs (180). IMU (accelerometer/gyroscope): detects phase onsets/offsets, transitions (ingress/egress), gait/steps, and device-motion artefacts; provides body-orientation cues that disambiguate occlusions. PPG/HR: supplies heart-rate and rhythm proxies that help disambiguate breath cadence when audio/visual cues are weak; supports exertion/effort context. Respiration bands: provide direct inhale/exhale/hold phase timing for breathing synchronization (614). Foot pressure/shoe IMU: identifies stance width/weight distribution, contact timing, and asymmetries, informing safety (e.g., knee-over-ankle checks) and transition smoothness.

Transport and ingestion. Wearables connect via BLE/Wi-Fi (through the device's RF stack) and stream time-stamped samples; the client normalizes units/sampling rates and aligns them to the video timeline. Dropouts are handled by short buffers and confidence down-weighting. Wearable packet dropouts are mitigated using short rolling buffers (e.g., 0.3-2.0 s) with time-stamp alignment: the fusion module interpolates within the buffer to align samples to the video timeline and marks values stale beyond a staleness limit, reducing confidence and falling back to video/audio-only estimation when gaps persist; this bounds latency while smoothing jitter.

Fusion (127) with visual/audio context. The fusion module (127) aligns wearable signals with recent frames/audio to: (i) refine phase segmentation (610) and boundary detection (611), (ii) compute transition-duration and cadence features that are robust to visual occlusion, and (iii) strengthen breathing-phase estimates (inhale/exhale/hold) when video or audio alone is noisy. The output is a set of fused features with confidences attached to the session state.

Consumers and outcomes. Breathing analysis (153): fused respiration/PPG/IMU rhythms help determine in-phase vs. off-phase breathing relative to movement (614) and can suggest resynchronization points. Client/DRS (200/250): uses fused context to time brief cues (e.g., just before boundaries), to decide when to defer detailed instruction, and to present camera/lighting tips when fidelity is low. AI agent (150): when policy permits, a summary of fused signals may accompany the visual/audio request so the cloud analysis can incorporate wearable context under the same privacy (485) and transport (500) controls.

Privacy and policy. Wearable IDs are replaced with pseudonymous session identifiers; only the minimal features required for analysis are retained. If policy restricts raw telemetry from leaving the device, the client shares derived summaries (e.g., breath phase times, cadence) rather than raw streams.

Robustness. If telemetry quality drops (bad fit, motion artefacts), confidence is reduced and the system may fall back to video/audio-only breathing estimation, with the DRS preferring camera/lighting hints over nuanced cadence tips until confidence recovers.

### Edge/cloud Partition and Caching (FIG. 17)

FIG. 17 depicts how, in some embodiments, the system partitions work between on-device (edge) processing (481) and cloud/server processing (482), and how privacy filtering/anonymization (485), encrypted transport (500), and compression/caching (486) are applied. The partition can change dynamically based on device capability, bandwidth, battery/thermal headroom, and mode (e.g., live coaching versus review). In certain embodiments the entire pipeline runs locally on the user's device (110) (no cloud/server is involved) and optional local devices (e.g., external cameras (195), wearables (180)) provide auxiliary signals to the device.

Elements (roles). 481: On-device (edge) processing. Capture; pre-processing; optional lightweight feature extraction; redaction (485); client-side selection/presentation (DRS, TTS, overlays). In local-only embodiments, 481 includes the full AI agent pipeline (pose/movement/breathing analysis, feedback generation/segmentation). 485: Redaction (anonymization). Face/ID masking or cropping; metadata minimization; pseudonymous session IDs; payload minimization (e.g., user-region crops). Applied before any network egress. 500: Encrypted transport. TLS/HTTPS or equivalent secure channel for requests/responses when the cloud is used; integrity protection of messages and model/package downloads. 482: Cloud/server processing. Model-heavy analysis (pose, movement sequencing, breathing, error classification), response generation and segmentation. (When local-only, 482 is omitted.) 486: Compression/Cache. Policy-controlled compression and caching of requests/responses and selected artifacts (e.g., reference templates, audio clips, overlay assets). May exist on device and/or in the cloud which is reflected in FIG. 17 by using dashed lines for the element 486 and its connections. 471-473: Segmented response classes. Returned content bundles (brief/detailed/additional) that the client's DRS uses to time and select presentation.

Partition policy (when both edge and cloud are available). A decision policy allocates tasks across 481 and 482 to meet latency and energy targets and to ensure responsiveness under intermittent connectivity. Inputs to the policy include device class and SoC resources (113 CPU/GPU, 114 NPU, 115 ISP), power/thermal (118), and network state (119/160). The policy can keep TTS/overlays local while offloading model-heavy analysis, or (on capable devices) run more of the pipeline on-device.

Data-flow semantics. Edge→Cloud (hybrid): Media/context prepared on device pass through redaction (485), then are sent over encrypted transport (500) to 482. Cloud→Edge (hybrid): 482 returns a multi-segment bundle (471-473) over 500; results and derivations may be cached (486) on server and/or device to reduce latency and bandwidth for follow-ups. Local-only (no cloud/server): 481 executes the entire analysis pipeline (pose/movement/breathing, feedback generation/segmentation). The device presents results directly; 500 is not used for off-device processing (though link-level security still applies for local peripherals such as BLE wearables). 486 can cache local results and assets for review.

In some embodiments of the technology disclosed in this patent document, the client device 110 uses one or several cameras (e.g., 120/121-126; and, where available, an external camera 195, wearables 180, and the microphone 130; note that in certain embodiments, any of the cameras 120/121-126 can be external to the device 110; note that in some embodiments, any of the cameras 120/121-126 can be internal to the device 110) to locate, identify, and track the user's body and body parts, estimate their relative positions and orientations over time, and produce an enhanced image or video artifact that is better suited for downstream analysis and presentation. After (optional in certain embodiments) privacy filtering/anonymization 485, the client either transmits this enhanced media to the AI agent 150 via encrypted transport 500 for cloud/server processing 482, or, in an all-local configuration, executes the same analysis stages on-device within 481 and produces the multi-segment bundle 471-473 for immediate presentation, optionally using compression/cache 486 for efficiency.

To form a robust spatio-temporal representation of the body, the client may combine classical computer-vision methods with modern learning-based techniques. Classical methods include foreground/silhouette extraction and contour tracking; background subtraction and person/part masking; active and deformable contours; template matching and part-based models, including pictorial structures and deformable part models with latent SVM or related discriminative learners; feature-descriptor pipelines such as HOG/edge/texture features for body part localization; dense and sparse optical flow, motion-field estimation, and feature-track/trajectory tracking for temporal coherence; gait analysis; and dynamic time warping to align observed motion against a temporal template. Geometry-centric methods include 2D keypoint and landmark detection (single- or multi-person, top-down or bottom-up), multi-view triangulation and bundle adjustment, PnP/EPnP for camera-to-body pose, and depth-map analysis, point-cloud fusion, voxel grids, and surface fitting for 3D structure. Parametric body-model fitting can be used to recover consistent, animatable pose (e.g., SMPL and its derivatives such as SMPL-X, SMPL-H, or STAR (and MANO/FLAME for hands/face)) combined with inverse kinematics and pose optimization under joint and shape priors, optionally with differentiable rendering for reprojection consistency. Inertial signals from wearables enable IMU-based tracking, inertial mocap, and sensor fusion with complementary, Madgwick, Mahony, or Kalman filters (KF/EKF/UKF) to stabilize orientation and resolve occlusions. Learning-based models may include end-to-end pose regression networks, stacked-hourglass and high-resolution CNN backbones, transformer-based pose and spatio-temporal models with attention over space and time, graph convolutional networks operating on skeletal graphs (including spatio-temporal GCN variants), and self-or weakly-supervised representation learning to improve generalization. The device chooses and composes from these families according to capability and policy; the outcome is a temporally consistent, confidence-weighted estimate of body and part locations, orientations, and kinematics.

As used herein, an enhanced image (e.g., a single frame) or enhanced video (e.g., a short, time-stamped clip) is any representation derived from and/or including the user's captured media that has been optionally (i) normalized (for example, by stabilization, exposure/white-balance adjustment, subject-centric cropping, geometric re-framing, or de-noise/de-blur), and/or (ii) augmented with additional information (for example, derived channels, measurements, annotations, or metadata such as depth, optical flow, segmentation/occlusion masks, keypoint/landmark heatmaps, confidence maps, 3D pose parameters, or overlay directives), and/or (iii) privacy-filtered (for example, through redaction, blurring, matting, pseudonymous identifiers, or background suppression), and/or (iv) transformed in other ways favorable to downstream processing or presentation. The enhanced representation may be provided in place of the original pixels, in addition to the original pixels, and/or as separate sidecar data, and may be used to make subsequent analysis and/or presentation more accurate and/or more efficient. No particular normalization, augmentation, privacy filter, or transformation is required; any subset may be used alone or in combination, and different subsets may be selected dynamically per device capability or policy.

For example, the client can recenter and stabilize the view, normalize exposure/white balance, and attach or embed derived layers (for example, aligned depth and surface-normal estimates, optical-flow fields, segmentation and occlusion masks, keypoint/landmark heatmaps, confidence maps, and, where available, 3D joints or parametric body-model parameters) together with sidecar metadata (e.g., timestamps, intrinsics/extrinsics for multi-view, wearable summaries, quality/context flags). These additions may be paired with pose-conditioned photometric adjustments inside the foreground mask (e.g., local contrast equalization near joints), background suppression by matting to a neutral backdrop, and render-ready geometric primitives (skeletal sticks, angle arcs, vertical “stack” lines, shoulder-height bands, directional arrows). To preserve model robustness, the client can retain the original RGB while delivering a companion enhanced copy and/or separate analysis channels; when a single tensor must be sent, the transform can be chosen to be reversible or annotated so the agent can discount it if desired.

Because the client already knows where the body and parts are, it can modify the pixels themselves in ways that raise signal-to-noise for the next stage. It is often helpful to amplify local contrast and mid-frequency detail at detected joints and along articulated contours (for instance, applying contrast-limited adaptive histogram equalization within small, pose-aligned regions around knees, elbows, and shoulders) so edges are sharper and limb boundaries are easier to parse. It also helps to suppress the background outside the person mask (or outside specific part masks) by desaturating, blurring, or replacing it with a neutral matte; this reduces false positives from clutter and simplifies occlusion reasoning. Where garments or patterns confuse part boundaries, the client can normalize chroma or re-tint foreground regions gently and consistently, or equalize illumination across the torso and limbs, so appearance cues are less variable frame-to-frame. The same pose knowledge supports drawing machine-readable lines, curves, and surface cues directly into a copy of the image or as a separate layer: thin ridges along arm and leg contours, angle arcs at joints, vertical/horizontal reference bands derived from the floor plane and gravity, and arrows indicating the direction of recommended rotation or translation. Depth or stereo can be used to flatten shadows on the body while preserving shading gradients that convey shape; motion cues can be used to de-blur limbs along their flow and to time-warp short bursts to a uniform phase grid, reducing jitter before phase and cadence estimation. These modifications increase signal-to-noise for the downstream model(s) and agent, improving robustness and throughput: foreground-biased contrast stabilizes keypoint and part classification; background suppression cuts spurious detections; pose-aligned edge cues improve landmark localization and geometric fitting; color/illumination normalization dampens nuisance variability; and temporally consistent, de-jittered clips produce cleaner phase boundaries and more reliable duration and breath-sync features. When a cloud agent expects natural images, the client can send both the unaltered RGB and the enhanced version or route the enhancements into auxiliary channels; when running the full pipeline locally, the enhanced view simply feeds the on-device pose, movement, and breathing analysis directly.

In a hybrid deployment, the enhanced media is minimized and anonymized 485 and sent under encryption 500 to the AI agent 150 in 482, which performs the heavy perception pipeline (pose 151, movement 152, breathing 153, error classification 154), composes feedback 156, segments 155 the results into brief/detailed/additional 471-473, and returns them over 500. Caching 486 may be applied in the cloud to accelerate repeated references/templates and on device to speed review playback and overlay reuse. In an all-local deployment, the device's edge module 481 continues from the enhanced media to run the entire pipeline on-device: pose estimation and 3D lifting or parametric fitting (with optional IMU fusion and optimization), movement-sequence analysis with phase segmentation 610, boundary detection 611, transition-duration measurement 612 and reference-timing comparison 613 (including dynamic ime warping (DTW)/cadence alignment against local templates or references from 170), breathing analysis 153 from chest/abdomen motion, audio or wearables to produce the breath-phase timeline 614, error classification 154 into minor/moderate/critical with direction and magnitude of correction, and feedback generation 156 plus response segmentation 155 to produce the multi-segment bundle 471-473 and overlay directives. The client's DRS 250/474 then schedules what to surface and when, and the presentation layer 270 renders text/audio and overlays on display 111 or AR/HMD 190; no cloud/server 482 or transport 500 is used in this path, though peripheral links (e.g., BLE) remain secured.

The element relationships in such embodiments can follow the same privacy-first order. Capture from cameras 120/121-126 and, when present, an external camera 195 and wearables 180 is ingested by the device compute blocks (113 CPU/GPU, 114 NPU, 115 ISP), fused as needed via the multi-sensor module 127, and transformed into enhanced media. In the hybrid path the sequence is “enhanced media→485→500→482→471-473→500→device for DRS/presentation,” with optional caching 486 at the server and locally; in the all-local path it is “enhanced media→481 (151-156/155)→471-473→DRS 250/474→270,” with optional local caching 486 for fast review. This approach both improves robustness today—by giving the agent or local pipeline a richer, normalized signal to work with—and anticipates a future in which user devices routinely execute the complete AI/ML analysis stack entirely on-device.

External/local sources (still local in the all-on-device embodiment). When present, wearables (180) and external/drone cameras (195) stream to the device; their signals are fused via the fusion module (127) (see FIGS. 16 and 27) and consumed by 481. This improves robustness for phase/boundary timing and breathing synchronization without requiring any server.

Privacy and integrity. The pipeline includes on-device anonymization/redaction (485) prior to egress, encrypted transport (500) for any networked path, bounded retention using caching (486), pseudonymous session IDs, and integrity checks for model/package downloads.

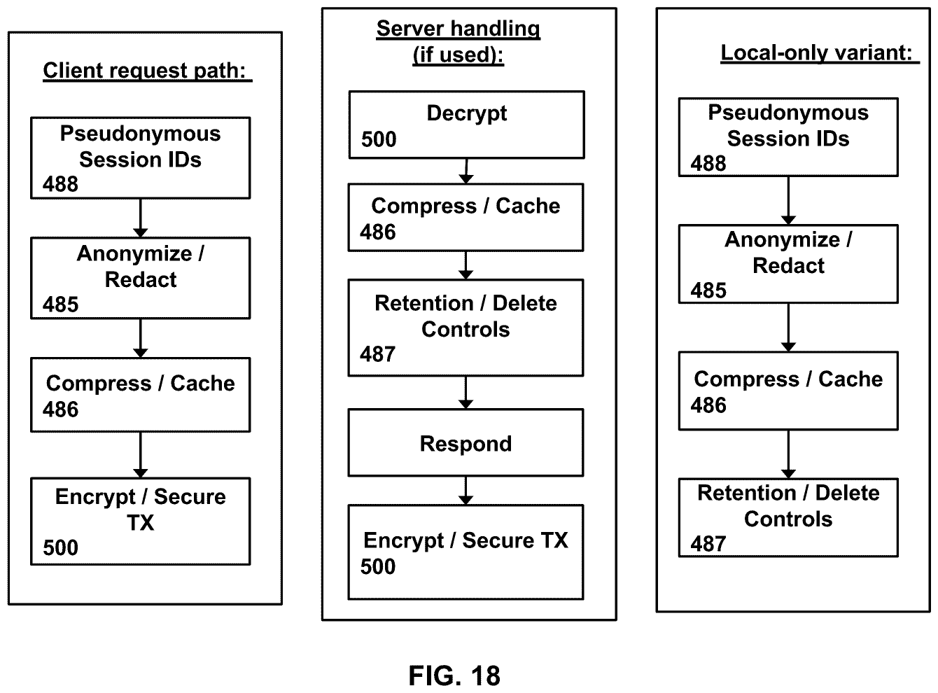

### Privacy and Data Lifecycle (FIG. 18)

FIG. 18 summarizes the privacy posture and data lifecycle across the client and any server components according to certain embodiments of the technology disclosed in this patent document: on-device anonymization/redaction (485) prior to egress; compression and/or caching (486) under policy; encrypted transport (500) for any networked exchange; and retention/delete controls applied to cached artifacts with association only via pseudonymous session identifiers. These measures implement data minimization, scoped retention, and end-to-end confidentiality/integrity, while allowing low-latency operation on device and, where used, efficient interaction with cloud services.

Anonymization/Redaction (485). Before any media or derived signals leave the device, 485 can remove or mask personally identifying regions and metadata. Examples include face/skin blurring or cropping, background matting, removal of EXIF (Exchangeable Image File Format) information and precise location, and pseudonymous session labeling in place of user identifiers. Where feasible, the request is reduced to subject-centric crops or derived layers (e.g., segmentation masks, keypoint heatmaps, depth/flow) in lieu of full-frame RGB to enforce data minimization without compromising analytical value. The same anonymization policy applies to auxiliary streams (e.g., sidecar JSON, wearable summaries).

Compression/Cache (486). 486 applies compression to requests and responses (e.g., image/video codecs, structured-data compaction) and controls caching at the client and/or server. Client-side caching (e.g., of recently synthesized audio for brief commands, overlay assets, or reference templates) minimizes bandwidth; server-side caching (e.g., reference timing templates, common overlay directives) reduces repeated computation. All caches observe retention windows and delete rules (see below) and are indexed by pseudonymous session IDs so they cannot be trivially linked to a real-world identity.

Encrypted/Secure Transport (500). All network exchanges use encrypted transport (e.g., TLS/HTTPS) with integrity protection and appropriate key establishment/rotation. Encryption happens after compression so payloads remain compact, and every hop in a multiparty topology (e.g., intermediary server) is protected. This applies equally to model/package downloads and telemetry upload.

Retention/Delete controls (487). Caches and logs (on device and/or server) are governed by explicit retention policies: short TTLs for transient artifacts; write-once, bounded logs where required; ephemeral identifiers; and delete-on-completion for sensitive payloads. Retention windows can differ by artifact type (e.g., brief-audio cache vs. overlay directives vs. reference templates) and deployment policy. User-visible settings may allow opting into longer retention for training history; the default can prefer minimal, purpose-bound retention.

Pseudonymous session identifiers (488). All request/response exchanges and cache entries are keyed to pseudonymous session IDs generated on the client (or by a policy-compliant broker) and not to real-world identities. Session IDs enable necessary association (e.g., joining a response to a request or attributing coach edits) without exposing personal identities, and they can be rotated or scoped per session, device, or time window.

Local-only embodiment (no cloud). In an all-local configuration, 485 and 486 still apply (e.g., to control what is stored and for how long) while 500 is not used for media transport (peripheral links like BLE remain secured). Retention/delete controls continue to bound the lifecycle of local caches and logs, and pseudonymous session IDs still label session artifacts so local review or export avoids direct identifiers.

### Security and Edge/Cloud Processing (FIG. 23)

FIG. 23 illustrates the security measures and processing partition between on-device (edge) components (481) and cloud services (482) according to some embodiments of the technology disclosed in this patent document. Latency-sensitive features (e.g., UI, TTS for brief commands, overlay rendering, capture-quality checks) may execute on-device. Before transmission, the client performs privacy filtering/anonymization/redaction (485) and then uses encrypted transport (500). In appropriate cases, data and results may be compressed and/or cached (486) under policy. The cloud (482) performs model-heavy analysis and returns results to the device for presentation.

Edge vs. cloud responsibilities. A decision policy allocates work across 481 and 482 to meet latency, battery/thermal, and bandwidth constraints and to maintain responsiveness under intermittent connectivity. Examples of edge-side work include: capture and pre-processing, on-device redaction (485), optional lightweight feature extraction, TTS and overlay rendering, and DRS-driven presentation. Cloud-side work includes pose/movement/breathing analysis and feedback generation/segmentation. The policy may adapt at runtime.

Privacy filtering/anonymization (485) prior to egress. Before any user media leaves the device, 485 can: (i) mask or crop personally identifying regions (e.g., face/other identifiers), (ii) remove or generalize metadata, (iii) replace user identifiers with pseudonymous session IDs, and (iv) down-scope payloads to the minimum necessary (e.g., user-region crops). These measures reduce privacy risk while preserving analytical value.

Encrypted transport (500). All network traffic uses TLS/HTTPS with session establishment, cipher selection, and integrity protection. This applies to requests to the AI agent and to responses returning to the device. Keys/sessions are managed per deployment policy.

Cloud processing (482) and return path. The AI agent (150) running in 482 performs the model-heavy pipeline (pose estimation, movement sequence analysis, breathing analysis, error classification, feedback generation, and response segmentation). The results (e.g., brief/detailed/additional segments and overlay directives) are returned over 500 for client-side selection and presentation.

Compression and caching (486). Under policy, 486 applies compression and caching to reduce bandwidth and improve responsiveness. In the FIG. 23 diagram the explicit connection 482→486 denotes server-side caching of results or intermediate artifacts to accelerate subsequent requests (e.g., repeated references/templates). Depending on deployment, client-side caching can also be used (e.g., recently presented audio or overlays) in accordance with retention settings.

Integrity and operational safeguards. The pipeline includes integrity checks (e.g., for model/package downloads), pseudonymous session identifiers, and hygiene steps that ensure only authenticated, untampered binaries and models are used. These measures complement 485/500/486.

Network topologies and key handling (cross-references). Intermediary server (FIG. 28). In some deployments, the secure request may traverse an intermediary for credential insertion or request normalization; the same 485→500 requirements apply on each hop. Edge/cloud partition (FIG. 17) and privacy lifecycle (FIG. 18)—these figures situate 481/482 in the broader system and detail data-lifecycle and caching controls that complement FIG. 23.

Data minimization and retention. Consistent with privacy filtering (485), the system favors minimal, purpose-bound payloads and bounded retention (e.g., automatic deletion or short TTLs (time-to-live) for caches), subject to user settings and policy.

### Network Topologies with Intermediary Server (FIG. 28)

FIG. 28 depicts network topologies according to some embodiments for routing requests from the client device (110) to the AI agent host (150), including (i) a direct path and (ii) a path via an intermediary server. The intermediary exists to handle credential management and request normalization without exposing long-lived secrets to the client; it may also apply policy controls and limited performance optimizations. In all topologies, the client can follow the security/data-lifecycle sequence: on-device anonymization/redaction (485) before egress, compression/caching under policy (486), and encrypted transport (500) over the network (160). When a model/pose store (170) is client-hosted, the client can include a selected reference alongside the user's media; when 170 is remote (e.g., co-located with 150), the agent can retrieve the required reference there.

Direct topology (baseline). In the direct configuration the device sends a redacted, compressed request from 110 over 160 to 150 using 500 (e.g., TLS/HTTPS). The agent performs analysis and returns a multi-segment bundle; the client's DRS then selects and presents the result. This topology minimizes hops and suits deployments where device-side credentials, regional routing, or API (Application Programming Interface) governance do not require a proxy.

Intermediated topology (e.g., with credential broker). In the intermediated configuration the device transmits the redacted, compressed request from 110 to an intermediary 165 over 160 using 500. The intermediary can, e.g., insert or mint short-lived credentials, normalize and validate the request, apply policy gates (authentication/authorization, rate limiting, safety rules), and forwards the request to 150 (again over 500). Responses flow back through the intermediary to 110. This arrangement keeps long-lived API keys out of the client, centralizes policy and observability, and allows gradual rollout or A/B of agent versions without changing the app. Where permitted, the intermediary may cache non-sensitive artifacts (e.g., public reference templates or common overlay assets) to reduce latency; privacy rules and retention windows apply.

Data and privacy flow (both topologies). Regardless of route, the client can enforce 485→486→500 (redact→compress/cache→encrypt) before media leaves the device. Association is maintained with pseudonymous session identifiers; server-side caches observe bounded retention. If the 170 store is local to 110, the request may include the selected reference (image/clip or parametric template); if 170 is remote, 150 acquires it. Downstream, the multi-segment bundle (471-473) returns to the client; the DRS (250) times and selects content; the presentation layer (270) renders on 111 or 190.

Local-only embodiment (no cloud hop). FIG. 28 also admits a local-only variant with no network path to 150: the edge module (481) on 110 runs the full pipeline (pose, movement, breathing, feedback/segmentation) and presents the result directly. Wearables (180) and external cameras (195), if used, connect locally; privacy and retention controls still apply to local caches, but 500 is not exercised for media transport.

### AR/HMD Overlay Embodiment (FIG. 24)

FIG. 24 depicts how, in some embodiments of the disclosed technology, guidance overlays are rendered in augmented-reality head-mounted displays (190) (or pass-through AR on a handset) using the presentation layer (270) and overlay renderer (360), with optional content coming from the AI agent (150). In certain embodiments, the agent supplies overlay directives (or content that implies them), the client resolves those directives in specific coordinate frames, and the presentation stack renders the result into the AR view.

Coordinate frames and transforms. To make overlays stable and meaningful in AR, the system uses three frames: a world frame (anchored to the environment), a device frame (attached to the HMD or camera), and a body frame (attached to the user). In practice, the world frame is established by the AR runtime's SLAM/visual-inertial tracker and, where available, augmented with range data (e.g., ToF or LiDAR) so that the floor plane and gravity vector are known and remain stable as the user moves. The device frame is derived from the headset's or handset's IMU and camera pose, providing a continuously updated transform between device and world. The body frame is estimated from the pose pipeline (e.g., skeletal landmarks, SMPL-like fits, or part-centric features) and is typically rooted at the pelvis or torso with consistent axes. With these three frames in hand, overlay directives can be expressed and converted as needed: a vertical “stack” line can be authored in world coordinates so it remains truly vertical; a UI badge can be device-locked so it follows the user's view; and a corrective arrow can be body-locked so it rotates with the limb being corrected. In an AR/HMD implementation, the client maintains the transforms body↔device↔world and resolves each directive into device view space for rasterization.

What the overlay renderer does. The overlay renderer (360) accepts a stream of directives (lines, arcs, markers, arrows, labels, angle glyphs) and renders them onto the live AR view with the correct anchoring, scale, and occlusion. World-anchored guides use the world→device transform so they appear glued to the room; body-anchored guides use body→device so an arrow remains attached to, e.g., an elbow as the user moves; device-anchored elements (e.g., a comfort-zone panel) live in HUD (heads-up display) space. HUD space denotes the device-anchored, screen-space overlay layer (192) whose elements remain fixed relative to the user's view; unlike world-anchored or body-anchored guides, HUD items do not track 191/193 but are rendered after projection as stable UI panels, indicators, or reticles. Depth-aware occlusion and ordering ensure that a guide hides correctly when a limb passes in front of it. Comfort policies (maximum on-screen density, minimum size, color/contrast, fade-in/fade-out near boundaries, eye-box/vergence constraints) are enforced in the renderer so the same directive set yields readable, comfortable AR across devices. The presentation layer (270) handles timing (e.g., when to surface a brief cue or a detailed overlay) per DRS decisions and pushes frames to 190.

Content sources (agent-supplied vs. client-derived). Overlay directives (or overlays themselves, or, in some implementations, full images or videos with embedded overlays) can come directly from the AI agent (150) (e.g., “draw a vertical line through the ankle and an arrow from the knee toward that line”) or be derived locally from returned labels/measurements (e.g., a “knee-over-ankle” predicate converted by the client into a world-anchored line plus a body-anchored arrow). In keypoint-optional embodiments, the agent may provide relational predicates and target descriptions without keypoints; the client still converts those into the appropriate anchors using its live body/device/world transforms. In the all-local embodiment (no cloud), the same directives are generated on-device by the client's analysis pipeline and passed straight to 360/270.

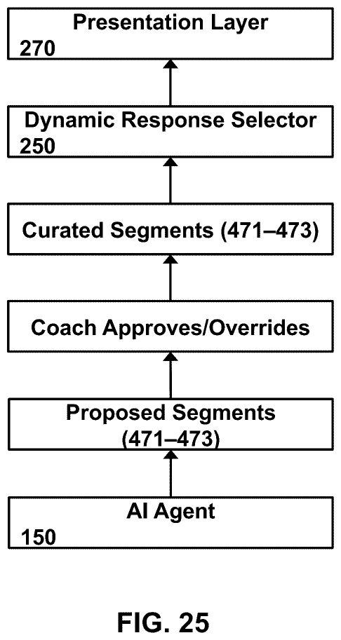

### Coach Co-Pilot/Override (FIG. 25)

FIG. 25 illustrates a coach co-pilot architecture according to some embodiments in which guidance generated by the AI agent (150) is optionally reviewed, reshaped, or augmented by a coach before presentation to the trainee via the Dynamic Response Selector (DRS 250) and presentation layer (270). The agent returns a multi-segment bundle (471-473; for example, brief commands (471) for imminent actions, detailed instruction (472) for learning and review, and additional information (473) for context). A coach, which may be an automated system, a robotic/smart-home component, or a human, can approve, edit, reorder, suppress, or insert items, with provenance recorded for each cue (“AI,” “Coach,” or “AI+Coach”). The DRS (250) then schedules what to surface when, using app state and phase-aware features, and 270 renders the selected items on the device display or AR/HMD.

AI coach (local or cloud). A coach can be another AI agent or subsystem that reviews the primary AI's bundle (471-473) in light of additional context (for example, prior sessions and goals, injury flags, device capabilities, available sensors/cameras, lighting/space constraints, or policy constraints). The AI coach may run locally on the client device (110) (e.g., a lightweight reviewer that enforces safety rules, house style, or brevity) or remotely (e.g., a richer meta-coach that has access to longitudinal history, team programming, or facility policies). It can (i) promote or demote items (e.g., elevate a safety-critical tip to brief), (ii) rewrite phrasing for clarity or tone, (iii) swap overlay emphasis (e.g., line→arrow), (iv) suppress redundant cues, or (v) insert context-specific content (e.g., “reduce range due to flagged knee.”). When timing is tight, the AI coach adheres to latency budgets tied to phase boundaries; if it cannot decide in time, the system falls back to the primary AI's brief selection to preserve real-time safety.

Robotic or smart-home coach. A coach can be a robotic system (e.g., a humanoid platform) or a smart-home component (e.g., a smart display with cameras, PTZ (pan, tilt, zoom) webcam, LiDAR/ToF depth, array mics, lighting, or haptics). Such a device may host the primary AI agent (150) that generates the bundle, and/or it may host the AI coach described above. With onboard sensors and actuators, the coach can adjust the environment (aim a PTZ camera for a better angle; brighten a light to improve exposure), demonstrate motions physically or via an avatar, and collect auxiliary views to strengthen analysis. Its review actions mirror the AI coach (approve/edit/reorder/suppress/insert), and its hardware allows context-aware interventions that non-actuated clients cannot perform.

Human coach (live or asynchronous). A coach can be a human who reviews AI-generated content. Humans are rarely able to make sub-second decisions reliably at transition boundaries; therefore, the system supports multiple interaction tempos. In a near-real-time mode (e.g., remote session), the human coach sees a compressed view of pending items together with phase timing and severity and can approve or nudge selections within a modest latency budget; safety-critical brief cues may bypass human gating. In an asynchronous mode (post-session or between sets), the human provides edits, insertions, and playbook rules (e.g., “always emphasize knee-over-ankle for this trainee,” “suppress complex hip cues until week 3”) that the DRS and/or AI coach apply automatically during subsequent live sessions. Humans can also author overlays or record or generate (e.g., using an AI) short clips to be inserted into detailed or additional segments, with provenance preserved.

Provenance, policy, and safety. Every segment carries a provenance tag identifying its origin (“AI,” “Coach,” or “AI+Coach”) and, optionally, a short rationale. Policy layers can enforce house rules (e.g., never suppress safety-critical cues; cap on-screen overlay density; language guidelines), and the system falls back to the primary AI's brief selection when the coach (of any type) misses its timing window. Privacy and security controls remain in force: where coach interactions traverse the network, redaction/anonymization (485) occurs before egress and encrypted transport (500) is used; caching (486) may store approved phrasing or coach playbooks under retention settings.

In operation, according to some embodiments, the client aggregates captured frames and metadata, performs lightweight pre-processing, and prepares requests carrying features and context. The agent performs higher-order reasoning, returns a response containing multiple segments that match different presentation contexts, and the client selects and renders the appropriate segment(s) (e.g., immediately or in a delayed or scheduled or contextualized fashion).

In some embodiments, the client application (200) includes a capture module (210) that acquires frames or (short) clips on demand or continuously, and a pre-processing module (220) that may normalize resolution, perform background suppression, or extract low-level keypoints. A request generator (230) bundles features with state and privacy metadata for transmission. A response processor (260) unpacks the agent's structured reply and prepares artifacts for the presentation layer (270), such as on-screen overlays or audio prompts. A state monitor (240) records whether the user is actively practicing, inspecting information, or idle/listening; a Dynamic Response Selector (DRS; 250) uses this state to choose between concise commands, detailed instruction, or supplemental information so that guidance remains timely and non-distracting.

This design reduces round-trips: the agent can return a collection of pre-composed segments in a single reply, and the client presents the one that aligns with the current context, while deferring others for later review.

According to some embodiments, the agent receives inputs (145) including images, video, and optional audio. A pose estimation module (151) outputs body keypoints and confidence measures that feed a movement analysis module (152) to evaluate temporal smoothness, joint sequencing, and phase alignment. A breathing analysis module (153) infers inhalation and exhalation cycles from chest/abdomen motion or audio spectral cues. An error classification stage (154) aggregates spatial and temporal features to label deviations from a target pose or sequence. A feedback generator (156) composes text explanations and corrective strategies and may attach overlay directives. A response segmenter (155) packages the content into brief commands suitable for in-motion cues, detailed instruction for learning phases, and additional material for context. The agent returns outputs (159) containing these segments and any overlay geometry. In other embodiments, the pose estimation module (151) and/or movement analysis module (152) and/or breathing analysis module (153) do not output body keypoints (as, e.g., keypoint coordinates or labels).

In certain embodiments, upon receipt of a response (600), the client performs at list one of: phase segmentation (610), detects phase boundaries (611), measures transition durations (612), compares timing to a reference (613), evaluates breathing synchronization (614), or classifies severity (615). In other embodiments, the client obtains from the AI agent's response or generates from the AI agent's response at list one of: phase segmentation (610), phase boundaries (611), transition durations (612), timing to a reference comparison (613), breathing synchronization (614), or severity classification (615). The Dynamic Response Selector (DRS) uses these signals, along with application state and runtime context (bandwidth, battery, confidence, quality issues (620)), to select one or more segments from the multi-segment bundle (brief commands, detailed instructions, additional information) for immediate presentation (270) or deferral.

An example: In a Vinyasa transition with an upcoming boundary in ˜300 ms and low bandwidth, the DRS selects the brief command ('step right foot forward') for immediate TTS, defers detailed instructions, and schedules a cadence cue to resynchronize breathing during the next hold phase.

Example DRS selection policy (priority ladder; non-limiting). Let B be time-to-phase-boundary; S be severity (minor/moderate/critical); Q be quality/context; C be connectivity/bandwidth; A be application state (practice, hold/review, idle/explore).

Priority 1—Safety & imminence. If (S≥critical) or (B<500 ms) in practice, present brief only (≤N items per the corrections-count), with TTS where available; preempt any ongoing detailed narration.

Priority 2—Hold/review windows. If A=hold/review and (Q is acceptable), present detailed plus overlays; include brief as bullet recaps at the end.

Priority 3—Explore/idle (reference browsing & determine-from-image). If A=idle/explore, prefer additional information (background/tips/tutorials) and navigation UI; optionally surface a compact pose-identification summary (e.g., detected pose name/description) only when, e.g., the user is browsing references or has invoked determine-from-image; otherwise suppress identification to avoid distraction.

Priority 4—Quality gating. If (Q=poor), substitute capture/lighting tips in place of form cues until Q improves; optionally suggest camera switching or repositioning.

Priority 5—Connectivity gating. If (C=constrained), prefer short text/TTS and defer video/long audio to cache; replay deferred items when C recovers.

Priority 6—Personalization. Apply user/coach policy (e.g., always elevate knee-over-ankle; cap cues during high-motion phases). Ties break toward safety then recency.

Preemption and coalescing. Near boundaries, the selector preempts ongoing low-priority narration and coalesces overlapping cues into a single minimal utterance, then schedules deferred details for the next hold. A hysteresis window prevents rapid toggling between brief and detailed near threshold crossings.

Hysteresis in selection timing. In some embodiments, the Dynamic Response Selector (DRS) applies hysteresis to phase-aware signals so that entry into a “near-boundary/brief-only” state occurs at a first threshold (e.g., B≤T_enter) while exit occurs only at a second, different threshold (e.g., B≥T_exit>T_enter) after a stability dwell. The DRS further enforces minimum on-screen dwell for overlays and cool-down windows for audio cue preemption. These measures prevent oscillation near phase boundaries detected by modules 610-612/611, reduce TTS preemption and UI churn, and yield predictable scheduling under tight latency budgets.

Audio preemption and cool-down. In some embodiments, the Dynamic Response Selector (DRS 250) applies audio preemption rules to the brief-cue audio path: a new, higher-priority or safety-critical brief may preempt (interrupt and replace) a currently playing or queued cue so that guidance lands before a detected phase boundary (600, 610-615). To prevent disruptive “thrash,” the DRS enforces cool-down windows after a cue starts and after it finishes during which equal-or lower-priority cues are not permitted to preempt; such candidates are queued, coalesced, or suppressed if their validity would expire. These measures, combined with hysteresis around boundary thresholds and intent debounce for voice/gesture inputs, stabilize the audio pipeline and reduce CPU/TTS churn while preserving hard preemption for safety-critical events.

Overlay density & comfort. In practice, show at most, e.g., one alignment guide and one directional arrow; in review, allow denser overlays. Density limits, minimum glyph size, and fade-in/out near boundaries are enforced to maintain readability and comfort. Here, “boundaries” are the temporal phase boundaries detected at 611 (onsets/offsets between ingress/hold/egress). The renderer, e.g., eases overlay opacity/scale (fade-in/out) within a small, non-limiting window around those events (e.g., ±100-800 ms) to prevent flicker and visual load; minimum glyph size and per-screen caps limit simultaneous guides during high-motion periods, while HUD-only elements (e.g., the brief guidance panel) remain legible without competing with body/world-anchored overlays.

Hybrid rule/ML operation. The selection logic may be rule-based, ML-based, or hybrid. In hybrid embodiments, rules enforce safety and timing constraints, while an ML ranker orders remaining candidates using recent behavior and preferences; if the ranker is unavailable, the rules alone decide.

Feature sources and fallbacks. If phase/timing features (610-614) and severity (615) arrive from the agent, the DRS uses them directly. Otherwise, the client derives functionally equivalent signals from returned labels/overlays and recent capture context without re-running cloud-grade models.

Personalization knobs. Thresholds for B (e.g., 500 ms default), maximum brief list length N, and overlay density caps are configurable, e.g., per user, device class, or mode, and can be adjusted automatically based on historical tolerance for interruption. “Historical tolerance for interruption” is a learned and/or user-configured preference derived from prior interactions indicating how much real-time interruption the user accepts (e.g., frequency of muting TTS, using pause/silence, swiping away or replaying cues, asking for more detail, or changing the corrections-count in Settings). The DRS may adjust thresholds such as B and the maximum number of concurrent brief items N based on this signal; the feature is optional, can be disabled, and can operate using only local aggregates without personal identifiers.

Provenance and explainability. Each presented item can carry provenance (AI/Coach/AI+Coach) and a short rationale when appropriate (e.g., “critical: knee ahead of ankle; boundary in 320 ms”), enabling audit and coach review without affecting real-time behavior.

In some embodiments, for single-frame or quasi-static analysis, a user frame (301) is evaluated relative to one or more reference poses drawn from a database (170). Keypoint or feature detection (310) yields joint positions that enable angle measurement (320) for joints such as elbows, knees, and hips. A deviation computation (330) calculates signed differences from target angles with confidence weighting; a thresholding stage (340) maps magnitudes to qualitative levels (e.g., minor, moderate, critical). An instruction generator (350) transforms deviations into concise, positively framed cues, while an overlay renderer (360) provides lines, arcs, and markers that visualize desired corrections on the display. These embodiments can be implemented entirely on the client (110) or using a combination of the client-side and/or AI agent (150) (or server)-side processing.

In certain embodiments, to handle occlusions, low light, or complex backgrounds, the system according to the disclosed technology may use different or multiple cameras, including, e.g., RGB (121), depth (122), infrared (123), LiDAR (124), time-of-flight (125), and thermal (126). A fusion module (127) combines these modalities, for example by aligning depth maps to RGB frames to stabilize joint estimates in poor lighting, merging thermal cues to monitor breathing in dim scenes, or using LiDAR for large-space calibration. The fusion module (127) can reside on the client side (110) or on the AI agent side (150) or both.

In some embodiments, a decision policy allocates processing between on-device (481) and cloud (482) based on device NPU capability, thermal state, bandwidth class, and battery level. On-device modules can include, e.g., pose/keypoint inference, privacy redaction (485), TTS, and overlay rendering; cloud modules can include, e.g., the instruction generator, multi-segment response generator (471-473), and model-heavy analysis, with cache layers and eviction policies to ensure responsiveness under intermittent connectivity.

The pipeline, according to some embodiments, includes on-device anonymization/redaction (485), compression/caching (486), encryption/secure transport (500), server-side retention windows, on-device delete controls, pseudonymous session IDs, and integrity checks for model/package downloads.

An embodiment of the disclosed technology can render overlays in head-mounted displays using world, device, and/or body coordinate frames, anchor placement on joints or floor plane, perform depth-aware occlusion, and use comfort-driven overlay density.

A coach view in some embodiments can approve/override and present the live feed, AI-proposed segments, suggestions, and/or other information or content; approved items are labeled with provenance when presented to the trainee, while the DRS adapts to coach inputs.

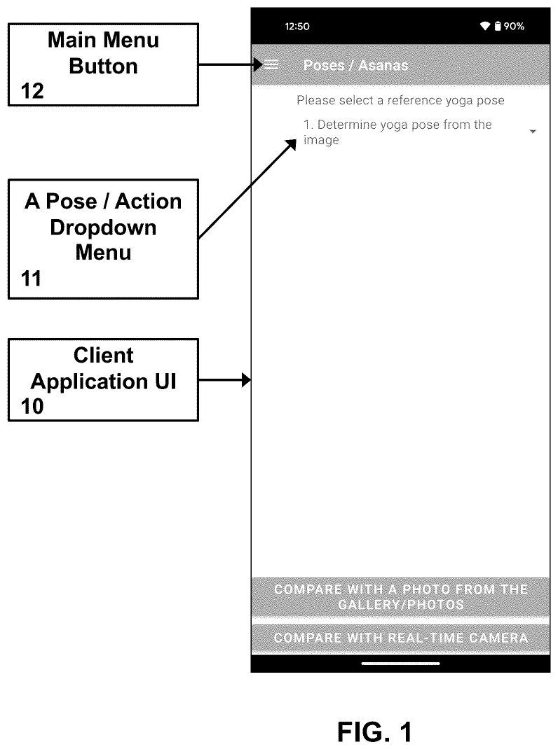

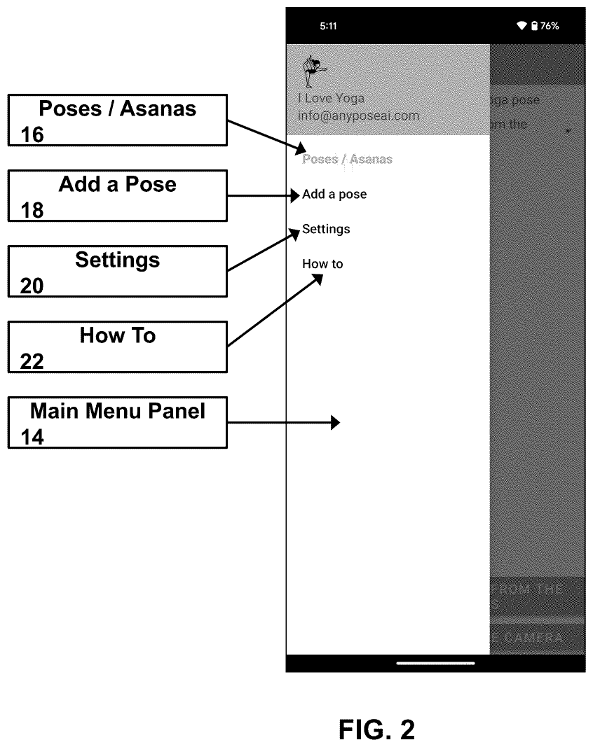

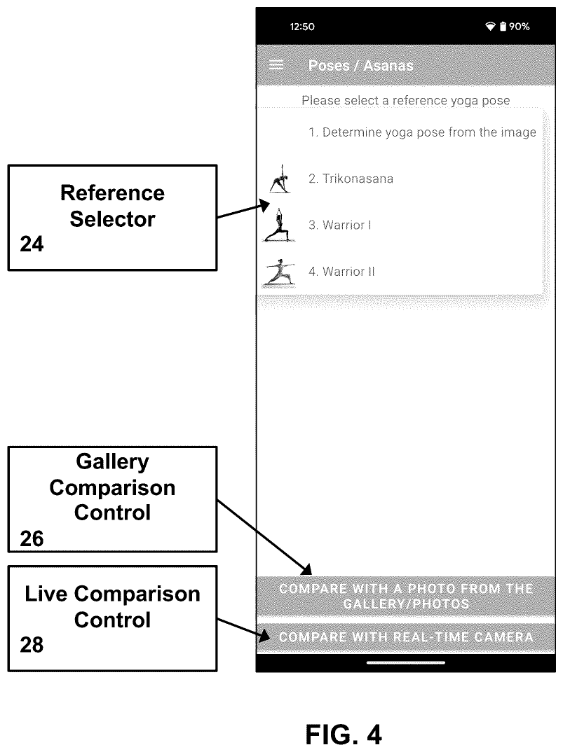

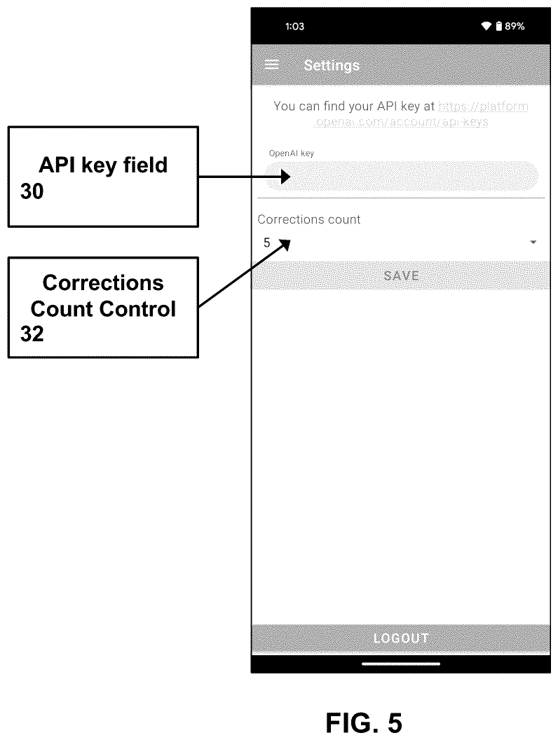

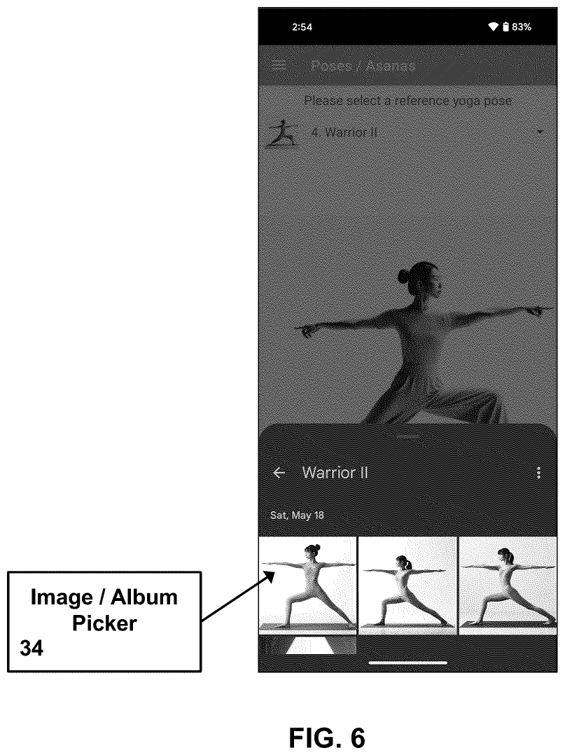

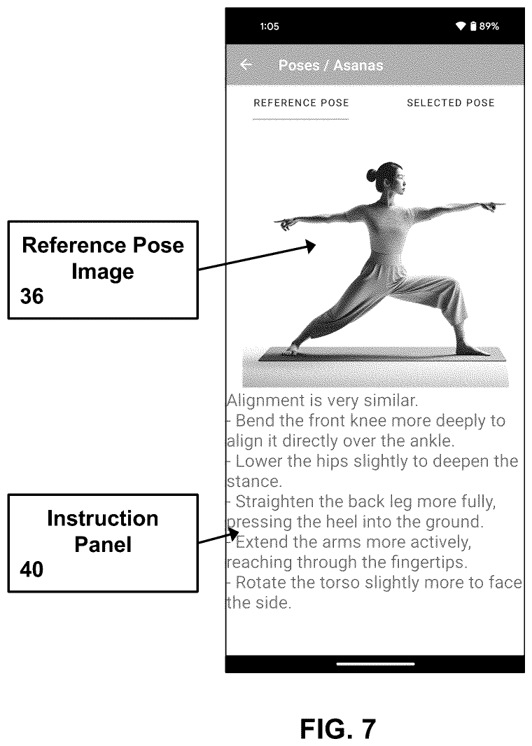

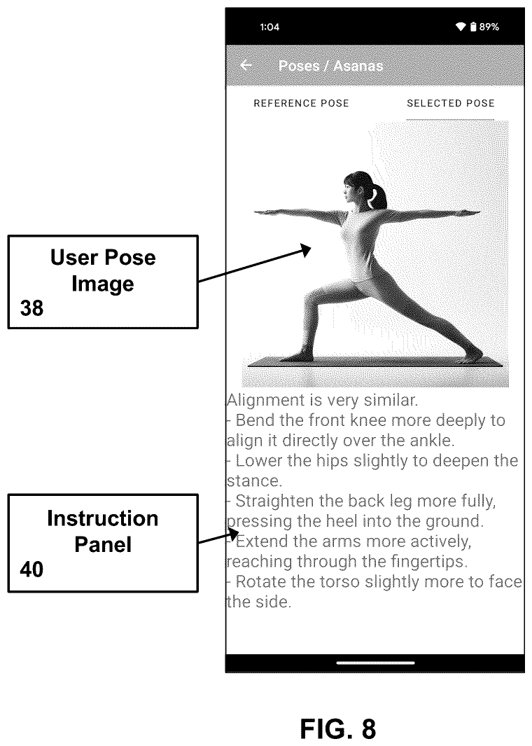

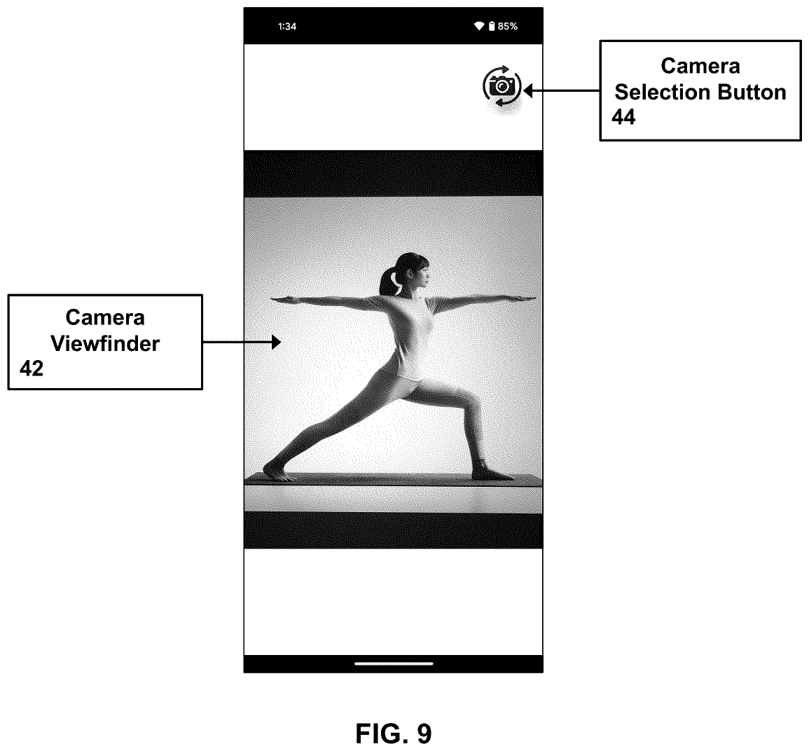

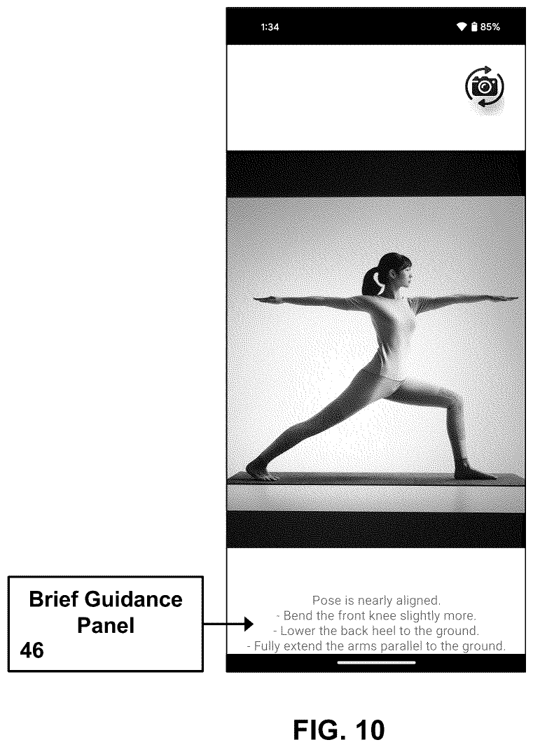

In a representative interface (FIG. 1), the application user interface (UI) (10) presents a main menu button (12) that expands to a panel (14) with Poses/Asanas (16), Add-a-Pose (18), Settings (20), and How-To (22) (FIG. 2). The Poses/Asanas view (FIG. 4) offers a selector (24) for a target reference, along with controls for gallery comparison (26) and live comparison (28). The Settings view (FIG. 5) includes an API key field (30) and a corrections-count control (32) that caps the number of concurrently displayed suggestions. An image/album picker (34) (FIG. 6) browses local or cloud sources. In examples (FIG. 7-8), a reference pose image (36) and a user pose image (38) are shown next to a generated instruction panel (40). A live camera viewfinder (42) with a camera selection button (44) (FIG. 9) enables hands-free voice-controlled operation; after capture, a brief guidance panel (46) (FIG. 10) distills essential cues (which can also be spoken out or vocalized by the app using text-to-speech functionality) for immediate action.

### Representative Yoga Session and DRS Behavior (FIGS. 1-10).

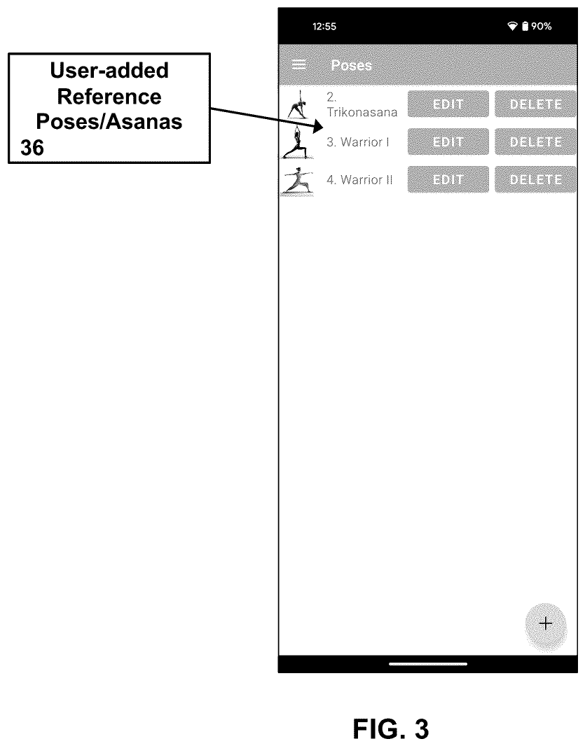

Representative session overview (FIG. 1-10). A user launches the client application and, on the main screen (FIG. 1), may use a pose/action drop-down (11) to (i) select a named asana, or (ii) choose “determine yoga pose from the image”. The user may also open the main menu (FIG. 2), which lists Poses/Asanas, Add-a-Pose, Settings, and How-To. From the Poses/Asanas screen (FIG. 4), the user can select (a) a curated reference pose, (b) a user-added pose previously created with Add-a-Pose (FIG. 3) (added poses later appear in the FIG. 4 drop-down), or (c) the determine-from-image option, in which the AI agent identifies the yoga pose from a single image captured live or chosen from the gallery. Before beginning, the user may set a corrections-count and other preferences in Settings (FIG. 5) and, when using existing media, select a photo or clip with the picker (FIG. 6). These screens establish the application state that the Dynamic Response Selector (DRS) uses when responses arrive, aligning presentation with what the user is doing now (reviewing, exploring, or practicing).

Gallery/review path (FIG. 6-8). When the user works from a gallery image or clip (FIG. 6) (including the determine-from-image mode) the app enters a review state. The returned bundle typically includes detailed instruction and brief commands. In this state, the DRS places detailed instruction beside the selected reference and/or user images (FIG. 7-8) and may add overlays, while keeping the brief items available as concise recaps. This lets the user study corrections without time pressure and return to practice later with a clear, prioritized list.

Live path (FIGS. 4, 9-10). Choosing Live Comparison (FIG. 4) opens the viewfinder (FIG. 9). During practice, the DRS favors brief commands with optional text-to-speech and a compact brief guidance panel (FIG. 10). As a phase boundary approaches, the selector surfaces a single high-value cue (e.g., “step the right foot forward”) and defers longer explanations until the next hold. When the user pauses, the selector can expand to richer text and overlays without disrupting flow.

Phase-aware timing and comfort. While the user moves, the client or agent derives phase segmentation, boundary timing, and transition durations; the DRS maps time-to-boundary and severity to a priority ladder that favors minimal, high-value cues in motion and richer narratives at holds. Overlays follow the same rule: e.g., thin alignment guides and a single arrow near transitions and denser measurement lines during holds or post-session review.

Quality/context gating. If the device detects low light, poor framing, or instability (quality/context flags), the DRS substitutes camera/lighting tips in place of form cues until fidelity improves. When bandwidth is constrained, the selector chooses brief text/TTS and defers video explanations; when connectivity returns, deferred segments can be presented or cached for post-session review.

Personalization and configurability. The corrections-count preference (Settings, FIG. 5) bounds on-screen density; the DRS also honors user or coach policies (e.g., always elevate safety-critical knee alignment). Over time, selector thresholds and phrasing can adapt to a user's tolerance for interruption and skill level, while preserving safety-first priorities.

Voice/gesture loop in practice (hands-free control; FIG. 9-10). In live sessions the app supports listening windows (short periodic intervals) or continuous monitoring for voice/gesture intents so the user need not touch the device (FIG. 9). Recognized intents (e.g., repeat last cue, more detail, pause guidance, next tip) are treated as state inputs to the DRS: repeated requests are coalesced so, e.g., the same cue is replayed once; a hysteresis window prevents rapid toggling between brief and detailed near phase transitions; and mute or pause holds selection until practice resumes. The brief guidance panel (FIG. 10) presents the currently active brief cue (or several brief cues) with simple affordances (e.g., a control (e.g., a voice-activated control) to replay the cue via TTS and an expand action to open the matching detailed instruction) so the user can confirm or deepen a correction without leaving the live view.

Post-session consolidation. After practice, the application returns to hold/review, elevating detailed instruction and annotated comparisons (FIG. 7-8) while offering optional tips or links under additional information. Cached segments enable offline review, and provenance tags indicate whether content was AI-generated, coach-curated, or both.

In some embodiments, UI implementations may adapt layouts to device orientation, support accessibility through screen readers and haptic feedback, and cache agent responses to make guidance available during intermittent connectivity.

In some embodiments, on-device models can include, e.g., lightweight 2D keypoint convolutional neural networks (CNNs), transformers, segmentation models, audio voice activity detection (VAD), keyword spotting, and/or on-device text-to speech (TTS). Cloud models can include, e.g., 3D pose lifting, transformer-based sequence models for timing and movements/breathing analysis, and/or multimodal Vision-Language Model (VLM) or Large Language Model (LLM) agents for image-text reasoning and instruction drafting. The architecture is model-agnostic and may select models dynamically based on device capability and context.

Vision-capable LLM operation without explicit keypoints in some embodiments. A vision-capable large language model can evaluate alignment directly from images by comparing visible geometry to textual targets (e.g., ‘front knee stacked over ankle; back foot near 90°; pelvis neutral'). The model identifies salient regions and axes, applies relative comparisons, and emits natural-language corrections and optionally overlay parameters (e.g., lines, arrows, angles) without explicit keypoint extraction.

In some embodiments: The agent may adapt target models to an individual's body proportions using a short calibration routine. The DRS can be personalized to a user's tolerance for interruption, medical restrictions, and skill level. Wearable sensors may supply heart-rate and respiration estimates to tailor pacing. The overlay renderer may operate in see-through AR glasses (190) to anchor cues in 3D.

### A System Overview

According to some embodiments of the disclosed technology, a client-side application and an AI agent are working in conjunction to deliver a comprehensive virtual fitness training experience.

A system according to some embodiments of the disclosed technology comprises several hardware components that are communicatively connected and working together:

### Video Camera

The system comprises a video camera which is configured to capture images or video of the user's poses or physical movements during exercise or yoga sessions. This camera can be an integrated component of a computing device, such as the front or rear camera of a mobile phone, tablet, or laptop, or it can be an external camera connected or communicatively coupled to the device.

The camera provides the visual data necessary for an AI agent to analyze the user's form and provide feedback. The video camera can be an RGB camera, an infrared (IR) camera, a depth-sensing camera (e.g., a structured light-based camera) providing depth maps, or a stereo camera for capturing multiple angles and/or creating 3D representations of the user's body.

The system may also use a LiDAR sensor for depth measurement or a time-of-flight (ToF) camera for advanced motion tracking and enhanced depth sensing. These sensors and cameras can work together with the AI agent to provide a detailed and precise analysis of the user's movements or pose alignment.

Additionally, thermal imaging cameras can be incorporated to provide temperature-based data, further enhancing the feedback in complex or low-light environments.

The inclusion of various types of cameras or sensors can facilitate adaptability to different environments and lighting conditions, improving the accuracy of the feedback provided by the system.

### Computing Device

The computing device may be a processor, mobile phone, tablet, laptop, or desktop computer. It is configured to run the client-side application, process the visual and audio data captured by the camera and microphone, and interact with the AI agent (which can be an independent or external AI agent or an AI agent incorporated into the client-side application) to generate real-time feedback.

The computing device's capabilities are enhanced by the disclosed technology, enabling it to perform complex analysis and deliver interactive training experiences. The computing device may include a graphical processing unit (GPU) to handle intensive image and video analysis tasks efficiently, improving the responsiveness of the real-time feedback mechanism. By optimizing the usage of both the central processing unit (CPU) and GPU of the computing device, the system is able to process high-resolution video and complex data while minimizing latency. Additionally, the computing device may handle gesture recognition, multi-angle video stitching, and real-time 3D pose estimation, ensuring smooth training experiences without performance bottlenecks.

### Microphone and Speaker

The system may also comprise a microphone configured to capture the user's verbal commands or breathing patterns, and a speaker configured to provide auditory feedback or instructions. These components further enhance the interactivity of the system, allowing for a more immersive and responsive training experience.

The microphone can be used for more precise breathing analysis, enabling the AI agent to synchronize breathing cues with movement patterns or movement cues with breathing patterns. The speaker can deliver real-time auditory cues, either through pre-recorded messages, synthesized speech generated by the AI agent, or text-to-speech conversion.

### Client-Side Application and AI Agent

The client-side application is configured to operate on a mobile device or computer, utilizing the device's camera and microphone to capture photos, videos, and audio of the user's physical activity, which is then sent securely to the AI agent for processing. The application functions in various modes such as real-time feedback mode, recording mode, and review mode.

The technical details of the client-side application include interfacing with the device's camera using application programming interfaces (APIs) to capture high-resolution images and/or videos, using audio APIs to capture audio commands and/or breathing patterns, and securely transmitting captured data to the AI agent using encryption protocols.

This secure transmission ensures user privacy and data integrity, particularly through the use of encryption algorithms like AES-256, along with secure communication channels such as HTTPS. Compression algorithms like JPEG2000 or WebP for images, VP9 or HEVC (H.265) for videos, may be employed to optimize bandwidth usage while maintaining high-quality data capture.

### AI Agent

The AI agent, according to certain embodiments, is a software component that is configured to process user data (e.g., photos, videos, audio) to provide real-time feedback and instructions. It can utilize various artificial intelligence or machine learning techniques and models to analyze and interpret the data.

The AI agent can be implemented using different technologies and architectures, such as neural networks, machine learning models, rule-based systems, transformers, or large language models (LLMs), and it can reside on a remote server, cloud server, local computer, or the same mobile device as the client-side application. In some implementations of the disclosed technology, the AI agent is embedded into the client-side application.

The AI agent can be configured to process incoming data by extracting key features of the user's pose alignment or movements, comparing them with stored, learned, or provided reference models, images, video, or data of ideal or target exercise execution, and generating segmented responses that comprise detailed instructions, brief commands, or additional information.

The AI agent can be configured to use machine learning (ML) models and/or image processing algorithms (such as, e.g., OpenCV, TensorFlow, or PyTorch) to analyze the user's data. The ML models can be trained on datasets that can include examples of both correct and incorrect exercise forms, movements, and/or alignment of different body parts such as limbs or joints.

The AI can be configured to recognize or be capable of recognizing (even if it was not specifically trained for that particular purpose) deviations in the user's performance from the ideal or target exercise form. Based on these deviations, the AI agent can generate feedback by applying predefined rules or using machine learning predictions to suggest corrections and improvements.

The AI agent can be configured to continuously improve the accuracy of feedback and instructions based on user interactions, utilizing, e.g., reinforcement learning techniques where the agent learns from user feedback and adjusts its models accordingly.

The AI agent can incorporate adaptive learning algorithms to personalize feedback based on the user's progress over time.

The AI agent can be designed with a modular architecture, allowing integration with third-party fitness tracking devices and sensors. It can employ multiple AI models to handle different aspects of data processing and feedback generation, ensuring comprehensive and precise feedback.

The AI agent can be implemented as a distributed processing system where different components are deployed across multiple servers or devices, increasing its resilience and security.

The AI agent can employ a hybrid architecture, where critical processing occurs in the cloud while real-time feedback is handled at the edge (on the user's device).

The AI agent can utilize proprietary data formats for transmitting and storing user data, adding a layer of complexity for anyone attempting to reverse-engineer the system. The AI agent can implement advanced security protocols, including multi-factor authentication and end-to-end encryption, to protect user data and system integrity.

The AI agent can be configured to dynamically adjust the level of feedback detail based on the user's current performance and historical data, ensuring a tailored and responsive experience.

### Instructions for AI Agent

The instructions for the AI agent can be categorized into four types, as an example, based, e.g., on their purpose and the stage of data processing or response generation they address.

First category: Instructions for processing information in client requests guide the AI agent on how to analyze the data or what metrics or features to extract from the data. Example instructions include posture (or asana) analysis, where the AI agent is instructed to analyze, e.g., the alignment of the user's head, spine, arms, and legs in the provided photo or video frames; or movement accuracy, where the angles of the user's joints (e.g., elbows, knees) are compared with the reference model or data, along with assessing the consistency and synchronization of the user's breathing with their movements.

The AI agent can be instructed to identify errors in static poses or dynamic sequences of movements by specifying, in a request to the agent, the type of pose or movement sequence being performed, defining the critical points (e.g., joints, limbs) and ideal angles or positions for these points, including information about the timing and synchronization of movements for dynamic sequences, outlining the acceptable range of deviations for each key point or movement, or classifying the severity of potential errors based on their impact on performance and safety. In some implementations, the AI agent can be instructed to identify errors in static poses or dynamic sequences without specifying these details.

Second category: Instructions for capturing new data direct the AI agent to capture additional data using devices like cameras or microphones when necessary, or to issue instructions to other software or agents (e.g., the client-side application) to capture such additional data.

For example, in real-time posture correction, if the user's posture deviates significantly from the ideal, the AI agent may activate a camera to capture additional angles for a more detailed analysis. The AI agent may also instruct the user to adjust their position relative to the camera.

Similarly, if inconsistent breathing patterns are detected, the AI agent might use the microphone to capture an audio clip (e.g., 30 seconds or 1 minute long) of the user's breathing; or, in low-light conditions, instruct the client device to turn on the flash or request the user to adjust the lighting and capture a new photo or video.

Third category: Instructions for processing the total volume of information guide the AI agent on how to handle the total volume of information, which includes the information provided to the agent in the initial request and information additionally captured or obtained by the agent, and/or how to generate comprehensive feedback using that total volume of information.

Example instructions include data integration, where the AI agent is directed to combine the initial video with additional captured angles (e.g., to create a 3D model of the user's posture); comprehensive analysis, where the integrated dataset is analyzed for overall posture accuracy, movement fluidity, and breathing synchronization; and error detection, where inconsistencies or errors in the pose alignment or movements captured in the combined data are identified and prioritized for immediate correction or subsequent detailed review.

Fourth category: Instructions for generating multi-segmented responses specify how the AI agent should generate the response to the client, detailing the format and content of the feedback. Example instructions include generating detailed text instructions for correcting the user's posture based on the analyzed data, creating annotated images highlighting areas of incorrect posture and suggesting adjustments, producing videos showing the correct execution of the user's movements with side-by-side comparisons, generating image or video overlays, or generating text or audio instructions or commands for real-time correction during the user's current exercise session. Segmentation of the AI agent responses is further discussed below.

The instructions for the AI agent can be structured to guide it through various stages of data analysis and response generation, ensuring accurate and contextually relevant feedback for the user.

These instructions can also be categorized, as another example, into several key components: pose identification and analysis, movement sequence analysis, data capture and processing, and feedback generation.

Pose Identification and Analysis focuses on identifying key points in static poses and analyzing their alignment with ideal or reference poses.

For example, in the Warrior II pose, the AI agent can be instructed to identify key points such as shoulders, elbows, wrists, hips, knees, and ankles; measure angles at each key point relative to the reference model; compare the user's key points and angles to the reference model; calculate deviation percentages for each key point; and flag deviations below 5 degrees as minor errors, deviations between 5 and 10 degrees as moderate errors, and those exceeding 10 degrees as severe errors.

In some embodiments (also referred to as implementations in this patent document), the AI agent is provided with instructions that ask it to compare pose alignments in two images, the one showing the user and another one showing a reference pose (e.g., a yoga pose or asana), without specifying how this comparison should be performed.

In some implementations, only the image showing the user is provided to the AI agent, and the AI agent either uses its internal knowledge about the proper pose alignment or obtains that information elsewhere to perform the user pose alignment analysis.

In certain implementations, the AI agent identifies the pose (e.g., the yoga pose or asana) the user is trying to perform using just the image of the user and without receiving this information in the user request.

The AI agent can perform key point identification and angle measurements itself or can use one or more specialized sub-agents to perform analysis of different pose alignment elements such as key point identification and angle measurements.

These sub-agents can have different architectures and can use different methods for pose analysis. For example, a sub-agent can use a convolutional neural network (CNN) for key point identification, another sub-agent might employ statistical models for measuring alignment based on predefined joint positions, while a third sub-agent could utilize support vector machines (SVM) for further refinement of the analysis.

Movement Sequence Analysis is aimed at analyzing the correctness, fluidity, timing, and synchronization of dynamic sequences.

For example, in the Sun Salutation sequence, the AI agent is instructed to segment the video of the user or a sequence of images that captures user movements into distinct phases such as Forward Bend, Plank, Upward Dog, and Downward Dog; identify the start and end points of each phase; measure the duration of transitions between phases; compare the timing of transitions to a reference model; check synchronization with breathing (e.g., inhale during Upward Dog, exhale during Downward Dog); and flag timing deviations exceeding 1 second as moderate errors and those exceeding 2 seconds as severe errors.

In some implementations, the instructions instruct the AI agent to evaluate the correctness of the movement sequence performed by the user without specifying how this evaluation should be performed.

In certain implementations, the request to the AI agent may contain instructions related to certain aspects of the movement sequence (or a static pose) but not the others, which can be covered in another request or not covered at all.

Data Capture and Processing focuses on capturing or requesting additional data if necessary and processing the total volume of information for comprehensive analysis.

For example, the AI agent can be instructed to activate the device's flash and capture a new video if poor video quality is detected; capture additional videos from different angles if dynamic movements are detected; capture audio using the microphone if detailed breathing analysis is needed; integrate initial and additional data into a unified dataset; use image processing techniques (e.g., ML-based or OpenCV) for comprehensive analysis; and apply data fusion techniques to combine visual and audio data.

In some implementations, the AI agent issues commands or instructions and/or makes requests to other agents, systems, or software to perform the actions mentioned above.

In some implementations, the AI agent can be instructed to determine which additional information would be helpful in the context of the user request and available data.

In certain implementations, the AI agent can ask the user to change their position or orientation relative to the camera.

Feedback Generation involves generating multi-segmented responses, which may comprise detailed instructions, brief commands, additional information and/or other segments.

For example, for detected errors, the AI agent can be instructed to generate detailed text instructions based on severity; create annotated images highlighting incorrect postures or movements; produce video overlays showing correct execution alongside user performance; and generate real-time text or audio cues for immediate correction.

The AI agent can leverage advanced AI models for context-aware and detailed feedback generation. For example, in posture analysis, the AI agent can use convolutional neural networks (CNNs) to detect posture misalignment, feed detected errors and context into transformer models like GPT-3, GPT-4, GPT-5, . . . , GPT-1000 or BERT, and generate context-aware feedback such as, for example, “Your front knee is slightly forward. Align it with your ankle to improve stability”, and integrate the text output with visual aids and/or audio cues.

Other transformer-based models like T5 and DistilBERT as well as models having different architectures can also be employed depending on the nature of the analysis required.

The AI agent can utilize advanced algorithms, including vision-capable transformers and/or large language models (LLMs), such as GPT-3, GPT-4, GPT-5, . . . GPT-1000, BERT, T5, or RoBERTa, to process the provided instructions and perform error identification.

For instance, in static poses like Warrior II, the AI agent can identify errors related to joint alignment, limb orientation, or body posture.

In dynamic sequences like Sun Salutation, it can recognize timing deviations, incorrect poses or pose elements, incorrect transitions between poses, or lack of synchronization with breathing patterns.

These errors may be classified as minor, moderate, or severe, depending, e.g., on their impact on performance or safety.

For example, if the AI agent detects that a user's back leg is bent during Warrior II, it may suggest straightening it, while in Sun Salutation, delayed transitions may prompt a recommendation for smoother, more fluid movements.

For example, in a static pose like Warrior II, the AI agent can analyze the alignment of the user's front knee over the ankle and the straightness of the back leg; check the horizontal alignment of the arms and the upright position of the torso; and flag deviations exceeding 5 degrees as moderate errors and those exceeding 10 degrees as severe errors.

If the AI agent identifies that the user's front knee is misaligned by 8 degrees and the back leg is bent slightly, it can generate feedback such as, e.g., “Align your front knee directly over your ankle and straighten your back leg.”

In a dynamic sequence like, e.g., Sun Salutation, the AI agent can identify the key phases of the sequence; analyze the correctness of the pose alignment or movement execution in each phase; analyze the fluidity and timing of transitions between phases; check the synchronization of breathing with movements; and flag timing deviations exceeding 1 second as moderate errors and those exceeding 2 seconds as severe errors.

If the AI agent, e.g., detects that the transition from Plank to Upward Dog is delayed by 2.5 seconds, it can generate feedback such as “Smoothly transition from Plank to Upward Dog, ensuring your movements are fluid and synchronized with your breathing.”

### Data Capture and Processing by the Client

In some implementations of the disclosed technology, the user can command the client application via voice or touch to capture photos or videos of their physical activity. The captured data is sent to the AI agent, which uses it to analyze the user's current performance and compare it with reference data, which may include ideal poses, movements, and/or breathing patterns.

In some implementations, instead of sending the whole captured image of a scene with the user, the client-side application can select the part of the image where the user is and send that portion to the AI agent instead of sending the entire image. Similarly, the video or video stream can be processed by, for example, the client-side application to generate another video or video stream in which frames capture only a certain area within the original video frames that encompasses the user. This video or video stream can be sent to the AI agent instead of the original versions.

The selection of the part of an image where the user is located can be implemented through the use of computer vision techniques, specifically object detection models designed to recognize human figures. When a user captures or uploads an image or video to the application, a pre-trained object detection model, such as one based on convolutional neural networks (CNNs), can be utilized to identify the presence of people within the content. The model processes the input and detects regions that likely contain human figures, outputting bounding boxes that represent the coordinates of each detected person.

In scenarios where multiple people are present in the images or video, additional methods can be employed to identify the specific area containing the user. One approach is to integrate facial recognition algorithms that compare the detected faces with stored facial data of registered users. By matching the faces in the image or video with the user's profile, the system can accurately determine which bounding box corresponds to the user. This process effectively selects the area where the user is located among multiple individuals.

Alternatively, the application can prompt the user to manually identify themselves within the image or video. This can be facilitated by allowing the user to tap or click on their image or by selecting their bounding box from the detected figures. This manual selection helps the system isolate the user's location when automatic identification is not feasible.

Once the user's area is identified, the corresponding bounding box can be employed to crop, highlight, or zoom into the portion of the image or video containing the user. This allows the system to isolate and focus on the relevant part of the content, distinguishing it from irrelevant parts such as the background or other people. In the case of videos, this process can be applied to each frame to continuously track and highlight the user throughout the playback.

This implementation ensures efficient selection of the user's region in images or videos that include multiple people. By combining object detection with user identification techniques, the system can accurately isolate the user's area, enhancing personalization and improving the overall user experience. The method effectively distinguishes the user from other individuals and irrelevant objects, allowing for targeted image or video processing specific to the user.

In addition to detecting the user's location in an image, user pose and skeleton identification can be implemented through pose estimation algorithms that recognize key points on the human body. These algorithms, based on deep learning models, are capable of predicting the positions of various body joints, including but not limited to shoulders, elbows, wrists, hips, knees, and ankles. Upon receiving an image or video frame, the system can apply the pose estimation model to detect these key points and map them onto the user's body. The key points can then be connected to form a skeleton structure, providing a visual representation of the user's pose. This skeletonization process allows the system to track the user's posture and body movements by drawing lines between the detected joints, creating a simplified but accurate representation of the user's pose and overall posture.

Both functionalities (user detection and pose/skeleton identification) can be efficiently integrated into an application using pre-trained models that are optimized for real-time performance. These models enable the app to deliver real-time user interactions, tracking body movements, identifying postures, and dynamically adjusting visual content based on the user's position and pose.

Technical details of data capture and transmission include data compression of captured images and videos using algorithms like JPEG for images, WebP for enhanced compression efficiency, and H.264, H.265 or VP9 for videos to optimize transmission speed and reduce bandwidth usage; and encryption of data using, e.g., AES-256 before transmission to ensure security and privacy. Furthermore, Transport Layer Security (TLS) protocols may be utilized to safeguard the transmission channels, preventing data interception during communication.

### Segmented Responses

The AI agent can generate responses segmented into different types of information, including, e.g., detailed instructions that offer comprehensive guidance on improving the user's performance; brief commands that provide short, actionable instructions delivered in real time during the user's exercise session; and additional information, such as where to purchase equipment seen in reference materials, providing commercial value.

### User Interaction

The client-side application executing on the system's computational device can continuously listen for voice commands or process discrete audio clips triggered by or synchronized with (periodic) signals (e.g., audio or visual ones), allowing users to interact with the application via voice, touch, or gestures.

Gestures can include both those made on the screen of the system's device (e.g., swiping or tapping) and those performed by the user “in the air” (e.g., hand waving, finger pointing, or arm movements) that are detected by the system's camera using computer vision algorithms.

These interactions enhance the user's ability to control and interact with the application hands-free.

The application can use contextual cues, determined by the user's ongoing activity, the state of the client-side application, the state of the computational environment in which the client-side application is executing, or user performance metrics or gestures, to display relevant segments of the AI agent's response, such as brief commands during exercises and detailed instructions when requested by the user.

Technical details of gesture recognition can include using computer vision algorithms from libraries like OpenPose or MediaPipe, OpenCV, or TensorFlow to detect and interpret user gestures in real time.

Voice recognition can be implemented using speech-to-text APIs like Google Speech Recognition, Amazon Transcribe, OpenAI's Whisper or GPT models (e.g., GPT-5, . . . , GPT-1000), or Microsoft's Azure Speech API.

Contextual cues may be determined, for example, through (real-time) analysis of user behavior, such as identifying repeated errors in movement or breathing patterns, prompting the system to present appropriate corrective feedback.

The described interactions are closely linked to the hardware components of the system. For instance, the camera captures the user's gestures, while the microphone records voice commands. The processor within the computing device then interprets these inputs using, for example, AI models and triggers the appropriate response, which is delivered via, e.g., the screen or speaker.

### Visualization and Feedback Presentation

The client-side application can display information on the device screen, including text instructions for readable guidance on exercise improvements; image overlays that visually highlight areas needing attention using color-coded indicators (for example, red could indicate areas of severe misalignment, yellow for moderate errors, and green for correct positioning); video demonstrations showing ideal exercise execution with interactive elements for deeper insights; and audio feedback providing verbal instructions and cues can be played back to the user.

The image and video overlays can be generated by the AI agent and provided to the client-side application as part of the AI agent response, or they can be produced by the client-side application itself using information from the AI agent response(s).

Technical details of the visualization features can include the use of graphics libraries such as OpenGL or Metal to render real-time overlays on images and videos, and interactive elements implemented through touch frameworks like Apple's UIKit for iOS or Android's View system.

The visualization of feedback can be linked to the device's display screen, which can be a mobile phone, tablet, or laptop.

Additionally, augmented reality (AR)-enabled devices like AR glasses may be used for real-time overlays, where corrective markers and alignment lines are displayed directly on the user's live video feed or the user's body.

### Technical Details Pertinent to Some Embodiments

The client-side application can employ speech-to-text conversion and natural language processing (NLP) algorithms to continuously listen for voice commands. In some implementations, the client-side application can listen to the voice input and recognize user commands using the audio information directly without converting it to text. This can be achieved, for example, through pattern recognition algorithms that match specific audio features to predefined commands, bypassing the need for text conversion.

The client-side application and/or the AI agent can use computer vision algorithms to recognize gestures made by the user, interpreting specific movements or signs as commands. The computer vision model can use, for example, a pre-trained dataset of hand gestures to identify the user's actions and trigger corresponding actions, such as, e.g., capturing an image.

The AI agent and/or the client-side application can have a modular architecture, allowing it to integrate various machine learning models and algorithms. For example, the agent can utilize convolutional neural networks (CNNs) for image and video analysis, and recurrent neural networks (RNNs) for processing audio commands.

For example, the AI agent can employ a CNN for analyzing video frames to detect posture deviations, and an RNN to process sequences of voice commands for consistency and context.

The AI agent can employ multiple AI models to handle different aspects of feedback generation, ensuring comprehensive and precise feedback.

The client-side application and AI agent (which, as noted, can be part of the client-side application, can be executing on a user device, or can be executing on a remote server) can utilize metadata tags to segment and categorize data, wherein each segment of a request to the AI agent or AI agent's response can be marked with identifiers indicating the type of information (e.g., detailed instructions, brief commands, commercial information) in it.

Metadata tags can include, e.g., timestamps, data type (text, image, video, audio), and content type (detailed instruction, brief command, commercial), ensuring that each piece of data is processed and displayed correctly.

The application can provide interactive feedback through touch-sensitive overlays on images and videos, allowing users to tap on highlighted areas to receive detailed explanations and corrective measures.

For example, when the user taps on a highlighted area of their posture in an image, the application retrieves the corresponding feedback from the AI agent and displays detailed instructions on the screen or presents them in audio form.

The system can optimize data transfer by compressing images and videos before sending them to the AI agent and can use caching mechanisms to store frequently accessed data, reducing the need for repeated network requests.

For example, video files can be compressed using the H.264 or H.265 codec before transmission, and frequently accessed data, such as standard posture models or images, can be cached locally on the device to minimize network latency.

The AI agent can continuously improve the accuracy of feedback and instructions based on user interactions, using reinforcement learning techniques where the agent learns from user feedback and adjusts its models or responses accordingly.

For example, a reinforcement learning model can use user feedback as training data to refine its posture correction algorithms, ensuring more accurate and personalized recommendations over time.

The AI agent's modular design allows integration with, e.g., third-party fitness tracking devices and sensors, enhancing the accuracy and richness of feedback generated by it by incorporating data from multiple sources.

For example, the AI agent can interface with heart rate monitors and motion sensors, providing a more comprehensive analysis of the user's physical activity.

The client-side application can support an offline mode, allowing users to download training sessions, which can include information about poses and movements, and receive real-time feedback without requiring continuous internet connectivity.

Users can download a set of training sessions while online, and the application can use pre-trained models stored locally on the device to provide feedback during offline sessions.

The client-side application can incorporate augmented reality (AR) features to overlay instructions and corrections directly onto, e.g., the user's real-time video feed or the user's body observed through, e.g., AR glasses or virtual reality (VR) glasses.

AR technology can be used to display, e.g., alignment lines, arrows, and corrective markers on the user's live video feed, guiding them to adjust their posture in real time.

The video can be displayed on the user's computing device (e.g., phone, laptop, etc.) or projected onto AR or VR glasses the user is wearing, allowing for an immersive and hands-free training experience.

In a scenario involving AR glasses, the user could view real-time guidance overlaid onto their actual environment, helping them adjust their form while maintaining full mobility.

The AI agent can be configured or capable of processing data (images, video, audio) and generating feedback for multiple users simultaneously in a group training scenario.

The AI agent can track and analyze the poses and movements of multiple users in a group session, providing individual feedback to each participant based on their performance.

The client-side application can provide a customizable interface allowing users to select specific areas of focus, such as flexibility, strength, or balance, for tailored feedback.

Users can set their fitness goals in the application, which then can adjust the feedback and training plans to prioritize exercises that target those specific goals.

Users can set specific training objectives within the application, such as improving flexibility or balance. The application can use these objectives to determine which feedback segments to emphasize. For instance, if a user aims to enhance balance, the application (together with the AI agent in some implementations) may provide additional detailed instructions on poses that target balance improvement, both during and after the session.

The client-side application or the AI agent can incorporate a privacy mode that anonymizes user data before processing to ensure privacy and security.

User data can be anonymized by removing personally identifiable information (PII) and/or using encryption protocols to protect data integrity during data transmission and processing.

The client-side application can support integration with social media platforms or specialized sites for sharing progress and feedback with friends or trainers.

Users can connect their social media accounts to the application and share their workout progress, achievements, and feedback received from the AI agent.

The AI agent or the client-side application can use predictive analytics to forecast user performance trends and provide proactive recommendations.

The predictive analytics model can analyze historical performance data to identify trends and suggest adjustments to the user's training plan to optimize future performance.

The client-side application can include a rewards system to incentivize user engagement and adherence to training programs.

Users can earn points and badges for completing workouts and reaching milestones, which can be redeemed for rewards or discounts on fitness-related products or the client-side application subscription plans.

The AI agent can simulate or take into account different environmental conditions or factors and suggest adjustments to the user's exercise routine accordingly.

The AI agent can adjust training recommendations based on conditions such as altitude, temperature, and humidity. This can be done, for example, to prepare users for different real-world scenarios.

The AI agent can detect and adapt to different exercise types (e.g., yoga, Pilates, strength training) and provide specialized feedback for each type.

The AI agent can, for example, use one or several machine learning models trained on various exercise types to recognize and adapt feedback based on the specific exercise being performed.

The AI agent or the client-side application can integrate biometric data from wearable or non-contact devices to enhance feedback accuracy.

The AI agent or the client-side application can, for example, use heart rate, calorie burn, or other biometric data from connected wearables to provide more personalized and accurate feedback.

The client-side application can include a training plan generator that adapts based on the user's progress and performance.

The training plan generator can use AI algorithms to create and adjust training plans based on the user's performance metrics, goals, and feedback received during workouts.

The AI agent or the client-side application can conduct (periodic) assessments to evaluate the user's progress and adjust feedback accordingly.

The AI agent or the client-side application can schedule assessments to measure improvements in posture, flexibility, and strength, adjusting training plans and feedback based on the results.

The client-side application or the AI agent can support multi-language feedback and instructions.

Users can select their preferred language in the application settings, and the AI agent can provide feedback and instructions in the chosen language.

### Improvements to Computational Devices

The technology disclosed in this patent document improves the operation of computational devices by optimizing data processing, resource utilization, and network usage.

Specifically, the disclosed technology enhances the functionality of the computing elements in the context of implementing a virtual yoga or fitness instructor application.

By segmenting responses and integrating multiple types of data (e.g., text, images, videos, audio) into a single response, the system reduces the need for multiple requests and responses between the client application and AI agent.

This optimization leads to reduced computational load on the client device, lower network bandwidth usage, and enhanced user experience through faster and more comprehensive feedback delivery. It can also lead to more efficient utilization of AI agent resources if generating multiple types of output (e.g., detailed text instructions, brief text commands, image overlays, audio cues, etc.) in a single AI agent response is computationally less demanding than multiple rounds of response generation, where only information of a certain kind (e.g., detailed text instructions) is generated in each response. This can also lead to lower monetary costs because each AI agent response typically requires the same input information, meaning that the same input tokens are spent each time a partial response is generated. Therefore, generating multiple partial responses results in more input tokens being consumed to generate the same diverse output information.

By receiving multi-segmented responses, which include various types of feedback (e.g., detailed instructions, brief commands, and commercial information) in a single transmission, the computational device reduces the number of network requests, thereby lowering latency and reducing resource consumption.

This approach also minimizes the amount of data that needs to be processed at any given time, leading to smoother real-time interactions and quicker responses.

Furthermore, the structured processing of these multi-segmented responses within the client-side application enables the system to prioritize the most critical feedback first (e.g., safety corrections), thereby optimizing user experience and device performance.

The disclosed technology enhances processing efficiency by utilizing edge computing for certain tasks, where critical processing occurs directly on the user's device rather than relying solely on remote servers or cloud resources. This approach reduces dependence on cloud infrastructure and decreases latency, which is essential for providing real-time feedback in a virtual fitness trainer application.

For example, the following scenario can be implemented when the AI agent or some of its modules are hosted on the user device, or in the offline mode where local AI agents optimized for the edge devices can be used instead of the more powerful cloud-based ones: The client-side application can perform immediate analysis of the user's posture and movements using on-device machine learning models. These models, such as pre-trained pose estimation algorithms, can, for example, identify key skeletal points and joint positions from the live camera feed. By processing this data locally, the application can provide instant feedback on the user's form, detecting misalignments or improper techniques without the delays associated with transmitting data to a server and awaiting a response. This real-time analysis enables users to correct their posture immediately, enhancing the effectiveness of their workout and reducing the risk of injury.

Similarly, the application can recognize user commands through voice input or visual gestures by performing speech recognition and gesture detection algorithms on the device. For instance, if the user issues a voice command like “capture pose” or performs a specific hand gesture, the application can instantly respond by capturing an image or initiating a new action. Processing these commands locally eliminates the need to send audio or visual data to a remote server, thereby reducing latency and preserving user privacy. This leads to a more responsive and secure user experience.

Additionally, the Dynamic Response Selector (DRS) mechanism disclosed in this patent document operates on the user's device to determine which segments of the server response are relevant based on the current application state and user actions. By handling this processing locally, the application ensures that feedback is contextually appropriate and delivered promptly, enhancing usability and efficiency.

Other critical processing tasks performed on the client device can include data compression and encryption of captured images or videos before transmission to the server. This optimizes upload times and bandwidth usage while enhancing data security without relying on external services. The application can also render augmented reality overlays on the user's live video feed using, e.g., the device's graphics processing unit (GPU), providing immediate visual guidance such as, e.g., alignment lines or corrective markers.

By managing these critical processing tasks on the user's device, the application reduces reliance on cloud resources and mitigates latency issues associated with network communication. This edge computing approach allows for immediate, real-time feedback essential for a virtual fitness trainer, enhancing the overall user experience. It ensures that the application is responsive, efficient, and capable of providing high-quality guidance during physical exercises, all while optimizing resource usage and maintaining data security.

By offloading certain tasks to the device's GPU, the system leverages parallel processing capabilities, which results in faster image and video analysis.

The integration of advanced AI models, including transformers and LLMs, into the device's operations allows for more sophisticated data processing without significant performance degradation.

The modular architecture of the AI agent and client-side application facilitates seamless integration and scalability, enabling the system to adapt to various hardware configurations and processing capabilities.

The use of compression algorithms and caching mechanisms optimizes network bandwidth and storage utilization, further improving the functioning of computational devices.

Security enhancements, such as multi-factor authentication and end-to-end encryption, ensure data integrity and protect against unauthorized access, thereby maintaining system performance and reliability.

The technology's ability to process and analyze data from multiple sensors and devices simultaneously demonstrates an improvement in computational efficiency and resource management.

By employing adaptive learning algorithms and predictive analytics, the system can anticipate user needs and adjust processing workloads accordingly, optimizing computational resources.

Technical field of improvement. The disclosed system improves the way a general-purpose computing device acquires, transforms, transports, selects, and renders multimodal data during real-time guidance. Rather than organizing content for display, the architecture constrains data structures, module boundaries, and execution order so that a commodity device performs fewer network round-trips, fewer CPU/GPU wakeups, and more deterministic scheduling under tight latency budgets. These are improvements to the computer itself, observable at the OS, networking, and graphics subsystems, independent of the subject matter of the guidance.

Multi-segment bundling+client-side selection reduces network and scheduling overhead. The AI agent returns a single, structured bundle containing brief commands (471) for imminent actions, detailed instructions (472) for learning/review, and additional information (473). A Dynamic Response Selector (DRS) (250) with selection logic (474) on the client consumes that bundle and selects segment(s) based on phase/timing signals (600, 610-615), quality/context (620), application state, and runtime constraints. Because multiple presentation modes are pre-materialized in one reply, the device avoids iterative “ask/answer” loops, reducing radio usage, context switches, and UI thrash even when the user switches states rapidly (live→hold→review).

Deadline-driven, phase-aware scheduling stabilizes the event loop. The DRS applies time-to-boundary, severity, and hysteresis rules so that only deadline-feasible items are spoken or rendered, with non-feasible items deferred. This prevents oscillation near phase boundaries and coalesces repeats (e.g., “repeat last cue”) to avoid TTS collisions, layout churn, and excess CPU wakeups that otherwise degrade responsiveness on mobile SoCs (system on chips). The result is a more stable event loop and predictable latency during high-motion phases.

Transformed input (“enhanced image/video”) is a computer-centric improvement to vision I/O. The client produces an enhanced representation before analysis/egress: subject-centric cropping/redaction (485), normalization, and derived channels (e.g., depth/flow/masks/confidence maps; overlay-ready primitives) that raise signal-to-noise, improve cache locality, and reduce downstream compute. This transformation changes the manner in which the computer processes pixels (via ISP/NPU/GPU pathways), yielding fewer false positives and lower per-frame cost for both local and remote inference.

Quality-driven recapture and multi-sensor fusion prevent wasteful compute. A quality loop (detect 620→trigger 621→integrate 622→fuse 623) steers capture (framing, lighting, sensor choice) and fuses RGB (121) with depth/IR/ToF/LiDAR/thermal (122-126) and optional wearables (180) to improve confidence before heavy analysis. By adapting the acquisition strategy and rejecting low-fidelity inputs, the device avoids expensive re-analysis on poor data, conserving CPU/GPU and uplink bandwidth while improving robustness.

Edge/cloud partitioning and back-pressure improve energy, thermals, and continuity. Latency-sensitive functions (TTS, overlays, DRS) remain on-device (481) while model-heavy analysis runs in the cloud (482) over encrypted transport (500) with compression/caching (486). Partition policy responds to network class and PMIC/thermal state (118/119) so the device meets real-time deadlines without thermal throttling. Graceful degradation (e.g., brief-only continuity, cached assets) maintains function during link loss, reducing reconnect churn and battery drain.

Graphics-stack improvements via directive schemas and multi-frame anchoring. Overlays are resolved via a directive schema into world/device/body coordinate frames (191/192/193) with depth-aware occlusion and ordering, density caps, and minimum glyph sizes enforced by the renderer. Anchoring in multiple frames reduces jitter and stabilizes AR overlays on mobile/HMD GPUs—a technical improvement to real-time rendering, not a mere presentation choice.

Deterministic reuse of objective guides reduces recomputation. Objective overlays (360) (e.g., angle arcs, stack lines, arrows) are computed once from measured or implicit geometry and reused across presentation states (e.g., live, hold, review). This design eliminates redundant geometry derivation when the user navigates screens and reduces compositing work per frame, improving UI throughput.

Privacy-first ordering reduces payload size and I/O cost without loss of function. The enforced order (on-device redaction/anonymization (485)→compression/cache (486)→encryption/secure transport (500)) shrinks payloads before encryption, enables cache hits on repeated artifacts (e.g., reference templates, audio cues), and ties association to pseudonymous session IDs (488). This optimizes the I/O stack (fewer retransmits; faster TLS handshakes with smaller records) while preserving end-to-end confidentiality and integrity.

Hands-free control with intent coalescing reduces interrupt storms. Voice/gesture handlers feed intent signals into the DRS, which debounces/coalesces commands and applies hysteresis around state changes. The result is fewer preemptions of TTS and fewer UI invalidations, producing tangible CPU and audio-pipeline stability benefits on constrained devices.

Structured agent outputs enable local, policy-bound timing without requery. Because responses adhere to multi-segment bundle structures with validity windows keyed to phase IDs, the client can preempt, defer, or replay content without contacting the server, which lowers latency variance and network dependency during real-time operation. This is a machine-readable protocol improvement, not a content change.

External/drone camera and wearables integration improves coverage with bounded compute. The system admits multi-camera arrangements and auxiliary sensors under a fusion API (127), allowing the device to switch viewpoints or ingest summaries without rearchitecting the pipeline. Calibrated handoffs and lightweight registration improve phase/boundary detection while keeping compute bounded by confidence-aware fusion.

Practical application and non-abstraction. The claimed improvements are tied to specific machines (camera arrays, ISP/NPU/GPU pipelines, AR/HMD displays, radios) and specific data structures/flows (471-473 bundles; 474 selection; 485/486/500 privacy-I/O chain; 610-615/620 timing and quality signals). The technical effects—e.g., reduced round-trip count, bounded on-screen density, deterministic scheduling, lower thermal load, stabilized overlays - cannot be performed as a mental process or with pencil-and-paper; they arise from particularized configurations of compute, memory, radios, and graphics on the device.

By constraining how data is packaged, scheduled, transported, and rendered, the disclosed system produces computer-centric gains—e.g., lower latency/jitter, reduced compute and radio use, stable AR graphics, and resilient operation under adverse capture and connectivity—that are independent of the particular instructional content and rooted in specific technical mechanisms set out in this document.

### Contextual Feedback Utilization

The present section provides examples illustrating how the client-side application can dynamically utilize different segments of the AI agent's response based on the application's state and user interactions. By highlighting specific scenarios, the benefits of context-driven feedback are emphasized, showcasing improvements in user experience and the effectiveness of the virtual fitness trainer.

### Example Scenarios

### Real-Time Exercise Session with Brief Commands

During an active yoga session, the user positions the computing device to capture their movements via the camera. As the user begins performing a sequence of yoga poses, the client-side application operates in a real-time feedback mode.

The application captures video of the user's movements and transmits the data to the AI agent. The AI agent processes the data and generates a multi-segmented response, which includes brief commands and detailed instructions.

Based on the application's current state-active exercise mode-the client-side application prioritizes brief commands from the AI agent's response. These brief commands are converted into audio cues and delivered to the user via the device's speaker.

For example, as the user transitions into the Warrior II pose, the AI agent detects a slight misalignment in the front knee position. The application immediately provides an audio prompt: “Shift your front knee slightly outward”.

This immediate, concise feedback allows the user to correct their posture without interrupting the flow of the exercise, enhancing the effectiveness of the session.

### Post-Session Review with Detailed Instructions

After completing the exercise session, the user enters a review mode within the application. The application retrieves the detailed instructions segment from the AI agent's response.

The user can view annotated images and videos highlighting areas that required correction during the session. For instance, the application displays an image of the user's Warrior II pose with overlays indicating the misaligned knee and provides textual guidance: “Ensure your front knee is directly above your ankle to maintain proper alignment and prevent strain”.

This detailed feedback allows the user to understand the nuances of their performance and plan for improvements in future sessions.

### Adaptive Feedback Based on User Actions

If the user struggles with a particular pose or movement, the application detects repeated errors through performance metrics. In response, the application adjusts the feedback strategy.

For example, if the user consistently misaligns their hips during the Triangle pose, the application may switch to presenting real-time visual overlays highlighting the correct hip alignment. The application might also offer a tutorial video focusing on hip alignment techniques, utilizing the additional information segment from the AI agent's response.

### Interactive User Control

The interactive user control mechanisms enable users to engage with the application seamlessly during their exercise sessions without the need to physically interact with the device, which may disrupt their flow or balance.

Voice Commands for Real-Time Adjustments. Users can issue voice commands to control various aspects of the application while exercising. For instance, the user can say, “Next pose”, to advance to the following exercise in their routine, or “Repeat instruction”, to hear the last guidance again. If the user is finding a particular movement challenging, they might say, “Modify pose”, prompting the application to provide an easier variation or additional assistance. For example, during a session, the user might say, “Show me the correct posture”, prompting the application to display a side-by-side comparison of the user's pose and the ideal pose.

Gesture-Based Controls. The application can recognize specific gestures captured by the camera to execute commands. For example, raising a hand might signal the application to pause or resume feedback, while a thumbs-up gesture could indicate that the user has successfully completed a pose and is ready to proceed. A wave of the hand might trigger the application to replay the last feedback segment or switch to a different exercise.

Customizable Command Sets. Users can customize the set of voice commands and gestures within the application settings. This personalization allows users to select commands that are intuitive and comfortable for them, enhancing the overall user experience. For example, a user might prefer saying “Hold on” instead of “Pause”, and can adjust the settings accordingly.

Integration with Wearable Devices. The application can integrate with wearable devices that include sensors capable of detecting subtle movements or biometric data. For instance, tapping a wearable device could serve as a command to advance to the next exercise, or a double-tap might signal the application to provide more detailed feedback. Additionally, if the wearable detects elevated heart rates, the application might suggest a rest period or adjust the intensity of the session.

Real-Time Feedback Modulation. Users can control the type and amount of feedback they receive in real time. By saying “More details”, the user can request more comprehensive instructions, whereas “Brief cues” could reduce the feedback to essential prompts. This allows users to tailor the guidance to their current needs and preferences without interrupting their practice.

Language and Accessibility Options. The application supports multiple languages for voice commands and feedback. Users can switch languages on the fly by saying commands like “Switch to Spanish”. Additionally, for users with hearing impairments, the application can provide visual cues and accept sign language gestures recognized by the camera for command input.

Session Control Commands. Users can manage their session using commands such as “Pause session”, “Resume session”, or “End session”. They can also adjust the duration of poses or exercises by saying “Hold for 30 seconds” or “Reduce time by 10 seconds”, allowing for dynamic control over their workout.

Feedback on Demand. At any point during the exercise, the user can request specific feedback. For example, saying “How is my alignment?” prompts the application to analyze the user's posture and provide targeted feedback on alignment issues. Similarly, “Check my breathing” could initiate an analysis of breathing patterns if the application, e.g., integrates with appropriate sensors or uses a microphone to capture breathing sounds.

Environmental Adjustments. The user can adjust environmental settings through voice commands to enhance focus and comfort. Commands like “Increase volume”, “Dim the screen”, or “Mute notifications” allow the user to modify the application's behavior without manual interaction. Also, if the application detects that the user is in a low-light environment through camera sensors, it might adjust the feedback presentation by increasing text size or contrast in visual overlays, ensuring visibility without requiring user intervention.

Exercise Selection and Navigation. Users can select different exercises or routines by saying commands such as “Start flexibility routine”, “Show balance exercises”, or “Begin meditation session”. This enables users to navigate the application's content dynamically based on their immediate interests or needs.

Logging and Progress Tracking. The user can log progress or make notes using voice commands like “Record today's session”, “Save this pose”, or “Add note: improve balance”. The application stores this information for future reference, aiding in tracking improvements over time.

Emergency Commands. In case of discomfort or an emergency, the user can say “Stop immediately” or “I need help”, prompting the application to pause the session and provide appropriate guidance, such as relaxation techniques or advising to seek medical attention if necessary.

Social Interaction Features. Users can engage in social features through voice commands, such as, e.g., “Share my progress with friends”, “Join group session”, or “Challenge a friend”. This integration enhances motivation and community engagement within the application.

Adaptive Learning and Personalization. The application can learn from the user's interactions and adjust future sessions accordingly. If a user frequently requests modifications to certain poses, the application might proactively offer alternatives or adjust difficulty levels in subsequent sessions.

Privacy and Security Controls. Users can manage privacy settings via voice commands, such as “Disable camera”, “Go offline”, or “Erase today's data”. This allows users to control their data and device interactions without navigating through menus.

Multi-Modal Feedback Requests. The user can ask for feedback in different formats, such as “Show me in 3D”, prompting, e.g., the application to display a three-dimensional model of the correct pose, or “Explain verbally”, requesting an auditory explanation.

Content Customization. Users can customize their experience by saying commands like “Add this pose to favorites”, “Remove this exercise from my routine”, or “Set reminder for morning practice”, tailoring the application's content to their preferences.

Performance Challenges and Gamification. The user can initiate challenges by saying “Start endurance challenge” or “Compete against my best time”. The application responds by setting up activities that incorporate gamification elements to enhance engagement.

Instructional Mode Switching. Users can switch between different instructional modes with commands like “Switch to silent mode”, “Enable detailed guidance”, or “Activate beginner mode”, allowing them to adapt the instruction level to their comfort.

Error Handling and Assistance. If the application does not understand a command, it can respond with “Could you please repeat that?” or offer suggestions like “You can say ‘Next pose’ or ‘Show instructions’”. This facilitates smoother interactions and reduces user frustration.

User Feedback Mechanism. Users can provide feedback on the application's performance by saying “Send feedback”, followed by their comments. The application can log this input for developers to improve the system.

The incorporation of diverse interactive user control mechanisms empowers users to manage their exercise sessions effectively without interrupting their flow. By utilizing voice commands, gestures, and integration with wearable devices, the application offers a hands-free, intuitive interface that adapts to individual user needs and preferences. This enhances the overall user experience, promotes engagement, and contributes to more effective and enjoyable physical training sessions.

In some implementations, some or all of the user control features described above can be implemented within the client-side application using user interface (UI) elements such as tabs, windows, menus, and text fields that respond to clicks or touch inputs.

This alternative approach allows users who prefer or require manual interaction to control the application effectively. For instance, users can tap on-screen buttons to pause or resume feedback, swipe to navigate between exercises, or use dropdown menus to select different workout routines.

### Benefits of Context-Driven Feedback

Enhanced User Experience. By delivering brief, actionable commands during active exercise, the application helps users make immediate corrections without disrupting their concentration. This hands-free, real-time assistance enhances engagement and enjoyment.

Efficient Information Delivery. Segregating feedback into segments allows the application to manage information overload. Users receive essential cues when needed and can delve into detailed instructions at their convenience, improving learning outcomes.

Personalized Training. The application adapts to individual user needs by analyzing performance metrics and adjusting feedback accordingly. This personalized approach increases the effectiveness of the training program.

Improved Safety. Immediate correction of posture and movements reduces the risk of injury. By focusing on critical errors during the exercise, the application promotes safer practice.

### Some Related Technical Implementations

The client-side application can utilize metadata tags in the AI agent's response to identify and categorize feedback segments. Each segment can be associated with context indicators that determine when and how it should be presented.

The application monitors the state of the user interface, user interactions, and performance data to make real-time decisions on feedback presentation.

The application can analyze factors such as exercise phase, detected errors, and user preferences to select the appropriate feedback segment.

For instance, during high-intensity movements where the user's focus is critical, the application may limit feedback to essential brief commands. In contrast, during slower-paced activities or pauses, it may introduce more detailed instructions.

### Integration with Hardware Components

The application leverages hardware components from both the computing device and wearable devices to enhance context awareness and adjust feedback dynamically.

The computing device (such as, e.g., a phone, tablet, or laptop) is typically placed on a tripod, stand, or stable surface to capture video of the user's practice area, such as, e.g., a room or designated exercise space. This setup allows the device's camera to have an unobstructed and comprehensive view of the user performing exercises or yoga poses, without the need for the user to hold or interact directly with the device during the session.

The camera serves as the primary source of information for the application, providing video input that captures the user's movements and posture. The camera can be an integrated component of the computing device or an external camera connected to the device via a physical connection or a communication link. For example, the system can use a camera mounted on a quadcopter or a drone for indoor or outdoor use. This setup can provide multiple viewing angles that can be adjusted quickly. Using a separate camera offers flexibility, enabling the application to accommodate various hardware configurations and optimize the viewing angle and coverage of the practice area.

In addition to the video data from a camera, the application can integrate data from wearable devices equipped with sensors such as accelerometers and gyroscopes worn on the user's body. These wearables provide supplementary data on movement intensity, orientation, and specific limb or joint motions, enhancing the application's ability to deliver precise and personalized feedback.

For example, if the wearable sensors detect rapid movements, significant changes in orientation, or specific gestures, the application may prioritize audio cues over visual feedback to prevent the user from needing to look at the screen, thereby reducing distractions and maintaining the flow of the exercise.

By combining the rich visual data from the camera with the detailed motion and orientation data from wearables, the application gains a comprehensive understanding of the user's movements and posture. This fusion of data sources allows for more accurate detection of posture deviations, timing inconsistencies, and movement patterns, enabling better context-driven adjustments and more effective feedback.

The hands-free setup provided by the disclosed technology ensures that users can perform exercises naturally and comfortably, without being encumbered by holding a device. This arrangement also facilitates the use of larger screens, such as tablets or laptops, which can display more detailed visual feedback when needed.

The flexibility in hardware configurations, including the use of external cameras and wearable devices, allows the application to adapt to different user environments and preferences. Whether in a spacious home gym or a compact living room, the system can be tailored to provide optimal coverage and feedback accuracy.

Overall, the integration of stationary computing devices, primary video channels, and wearable sensor data enhances the application's ability to deliver immersive, context-aware, and effective virtual fitness training experiences.

The following sections describe a specific embodiment of the disclosed technology, including the implementation of a dynamic response selector (DRS) mechanism within the client-side application. This mechanism is designed to intelligently manage the interaction between the app's state, user inputs, and server responses, ensuring that the feedback provided is contextually relevant and optimized for real-time use.

### Additional Implementation Details

A client-side application according to some embodiments comprises components integrated into it or configured to be communicatively coupled to it, wherein the components are responsible for tracking or determining the application's state and/or user actions. These components can also interact with a server, forming and sending requests to it and/or processing server responses.

A server is a computer or system that provides resources, data, services, or programs to other computers, known as clients, over a network. A server according to the disclosed technology can be a cloud-based or remote server or a local server. The server can include hardware or software components or both. The server can be part of an application according to the technology disclosed herein. Note that we can refer to a ‘software application’ as an ‘app’ in this patent document.

A state of an app, according to some embodiments, can be any app state or configuration characterized by a combination of the user interface controls (e.g., buttons, lists, dropdown menus, tabs, text, images, etc.) and/or their states and/or their functions, or by a combination of one or more functions or services the app is configured to perform.

For example, the app can be in a state where it is configured to process an audio stream that can contain user voice commands, respond to those commands, and return to the audio stream processing. In another state, the app can be configured to allow its users to change its operational parameters or parameters or features of the app's user interface elements. In yet another state, the app can be configured to allow its users to select the name of a yoga pose from a dropdown menu and select whether the user desires to work with live images from the phone's camera or with the saved ones in the phone's ‘camera roll’ or image ‘gallery’.

An example method according to some embodiments can be implemented by a system configured according to an embodiment disclosed in this patent document and can comprise one or more of the following steps:

### Request Generation

Generate a request to a server that can include an image or video as well as text and/or audio. The image or video can be:

An image or video obtained by an app from a camera when the app is in a first operational state.

An image or video taken from the ‘camera roll’ or ‘gallery’ or ‘photos’ of the mobile device the app is running on. The device can be a computing device such as a phone, tablet, laptop, or computer.

An image or video of the app user.

### Response Reception

Receive a response from the server. The response can contain various parts or segments, each of which can be relevant to one or more states of the app and/or user actions.

### Segment Selection and Presentation

If the app is still in the first operational state when the response is received, present a first part or segment of the response via text and/or audio and/or images and/or video.

After the app switches from the first state to a second state following a ‘switch’ or ‘stop’ or ‘more info’ or another command (e.g., a button click or a voice command) from the user, present a second part or segment of the server response.

If the app switches from the first state without receiving a server response in the first state, it transitions to a third state upon receiving the ‘switch’ or ‘stop’ or ‘more info’ or another command.

If the server response does not include the first part or segment, the app may present the whole response or a third part or segment of the server response or a special message when the app is in the first state and/or after it transitions to the second state.

A key aspect of the disclosed technology is the presence in the server response of different segments or parts that contain information or data (e.g., in the form of text, images, video, or audio) providing different levels of detail for presentation to the user. For example, one segment of the response can include text providing a brief description or instructions to the user, while another segment can include text in which this description or instructions are presented in a more detailed way; this segment can also include additional descriptions or instructions not part of the first segment.

The description, instructions, and any other type of information contained in a response segment can incorporate various forms of presentation—from text to images to video or audio—as well as various combinations of these forms. A server response according to some embodiments can be described as having a hierarchical structure with different levels providing different degrees of detail or amounts of information.

A system according to some embodiments of the disclosed technology can choose which part(s) of the response to process or present to the user using information about the states of the app at various points in time, as well as information about user actions in the app or in the operating system of the device the app is running on at those or at different points in time, and/or information about the “real world” user actions.

According to some embodiments of the technology disclosed herein, decisions about which part(s) of the server response the app will process and use (e.g., use for presenting to the user) can be made by a Dynamic Response Selector (DRS) mechanism within the app. The DRS, according to some embodiments, is software logic, code, and other software and/or hardware mechanisms configured to determine the state of the application as well as the user actions (e.g., user interactions with the app via voice or image/video and/or its user interface elements) at the moment or after the server response was received in the app. The DRS can also use the state(s) of the app and/or user actions related to the app or made by the user in the operating system of the device the app is running on or made by the user in the “real world” (e.g., the exercises or movements the user was performing or the posture the user was holding, etc.) before the server response was received in the app.

### Some Example DRS Implementations

Rule-Based System: A rule-based system where predefined rules determine the response segment based on the app's state and user actions.

Machine Learning Model: A machine learning model that predicts the most relevant response segment using historical data on app states and user interactions or actions.

Hybrid Approach: A hybrid approach combining rule-based logic with machine learning to adaptively select the best response segment based on changing app contexts and user behaviors or actions.

### An Example Dynamic Response Selector (DRS) Utilization Scenario

The Dynamic Response Selector (DRS) is a core component of a software application according to some embodiments of the disclosed technology designed to dynamically process and present relevant segments of a server response based on the current and/or prior application's states and/or user actions. This mechanism enhances the application's efficiency and user experience by optimizing resource usage and providing contextually appropriate information.

### Technical Details

### Component Architecture

### Client-Side Component

State Monitor: Tracks the application's state and user actions.

Request Generator: Sends requests to the server based on user inputs and actions.

Response Processor: Receives complete server responses and interacts with the DRS to determine relevant segments.

Presentation Layer: Displays the selected response segments to the user.

### Server-Side Component

Response Generator: Processes client requests and generates comprehensive responses containing multiple segments.

Response Segmenter: Breaks down the comprehensive response into distinct segments based on predefined criteria.

### Operational Flow

### Request Generation

The application generates a request to the server, which may include images, videos (captured by the device's camera or selected from the device's gallery), or other data.

### Server Response

The server processes the request and generates a complete response with multiple segments, such as brief commands, detailed instructions, and additional (e.g., commercial) information.

### Dynamic Selection

The DRS evaluates the application's state (e.g., the app is listening for commands or presenting information) and user actions (e.g., voice commands, button clicks) to determine which segments of the server response are relevant.

The DRS uses a selection algorithm, which can be rule-based, machine learning-based, or a hybrid approach, to dynamically select the appropriate segments.

### Presentation

The application presents the selected segments to the user, adjusting the content based on real-time interactions and state transitions.

### Optimization Techniques

Context-Aware Processing: The DRS ensures that only relevant parts of the server response are processed and presented, reducing computational load and improving response times.

Resource Management: By handling only the necessary data segments, the DRS optimizes memory and CPU usage, which is crucial for mobile devices with limited resources.

Adaptability: The DRS adapts to changing application contexts and user behaviors, providing a seamless and intuitive user experience.

### Some Example DRS Implementations

### Example Implementation 1: Rule-Based Dynamic Response Selector System

Overview: In this example implementation, the DRS is realized through a rule-based system. The DRS utilizes predefined rules to dynamically select relevant segments of a server response based on the application's state or user actions. This approach leverages straightforward conditional logic to provide contextually appropriate information to the user.

### Technical Details

### Application States and User Actions

The application defines several operational states, such as “listening,” “presenting information,” and “idle.”

User actions are identified, including voice commands (e.g., “take a picture”), button clicks (e.g., “more info”), and gestures.

### Rule Sets Definition

A set of rules is established to map specific application states and/or user actions to corresponding response segments.

### Example Rules

Rule 1: If the application is in the “real-time practice” state when a response from the server is received, select and present the “brief commands” segment.

Rule 2: If the application is in the “presenting information” state and the user clicks “more info,” transition to the “detailed information” state and present the “detailed instructions” segment.

### Integration of Rule Engine

A rule engine (e.g., Drools) can be integrated within the DRS to manage and execute the predefined rules.

The rule engine continuously evaluates the current application state and user actions against the rule set to determine the appropriate response segment.

### Example Response Processing Workflow

Upon receiving a server response, the rule engine evaluates the rules to identify the relevant segments based on the current state and user actions.

The application then presents the selected segments through its presentation layer, ensuring that the user receives contextually appropriate information.

### An Implementation Example

If the app is in the “listening” sub-state within the “real-time practice” state and a user says “take a picture”, the application captures an image using the device's camera, sends a request including the image to the server, and returns to the “listening” sub-state.

The server processes the image and returns a complete response with segments such as, e.g., “brief commands”, “detailed instructions”, and “additional information”.

The rule engine evaluates the rule set and determines that, in the “real-time practice” state and when the most recent user command is “take a picture”, the “brief commands” segment is relevant.

The application presents the “brief commands” segment to the user.

If the user then clicks the “more info” button, the rule engine transitions the application to the “detailed information” state and presents the “detailed instructions” segment.

### Example Implementation 2: Machine Learning-Based Dynamic Response Selector System

Overview: In this example implementation, the DRS is realized using a machine learning model. The DRS leverages historical data on application states and user interactions to predict the most relevant response segments.

### Technical Details

### Data Collection and Preparation

Historical data on application states, user actions, and corresponding response segments are collected and stored in a structured format.

Data includes features such as, e.g., the current application state, type of user action, timestamp, and user preferences.

### Feature Engineering

Relevant features are created from the collected data to train the machine learning model.

### Model Training

A suitable machine learning algorithm (e.g., decision trees, random forests, neural networks) is selected for the classification task.

The data is split into training and testing sets to train the model and validate its accuracy.

The model is trained to predict the most relevant response segments based on the input features.

### Model Integration

The trained model is integrated within the DRS system.

During runtime, the model predicts relevant response segments in real-time based on the current application state and user actions.

### Continuous Learning

The system implements mechanisms for continuous data collection and periodic model retraining to improve prediction accuracy over time.

### An Implementation Example

When a user says “take a picture”, the application captures an image and sends a request, incorporating the image, to the server.

The server processes the image and returns a complete response with multiple segments.

The machine learning model evaluates the current app state (“exercises in real time”) and the most recent user action (“take a picture”) and predicts that the “brief commands” segment is most relevant.

The application presents the “brief commands” segment to the user.

If the user clicks “more info,” the model predicts that the “detailed instructions” segment is now relevant and transitions the application to present this segment.

### Example Implementation 3: Hybrid Dynamic Response Selector System

Overview: In this example implementation, the DRS is implemented through a hybrid approach, combining rule-based logic with machine learning. This approach leverages the simplicity of rules for straightforward scenarios and the adaptability of machine learning for complex user behaviors and contexts.

### Technical Details

### Basic Rule Definition

A set of basic rules is defined to handle common and straightforward scenarios.

### Machine Learning Model Training

Historical data on application states, user actions, and response segments are collected and used to train a machine learning model.

### Hybrid Logic Development

A decision-making layer is developed to dynamically choose between rule-based logic and machine learning predictions based on the context.

### System Integration

Both the rule engine and the machine learning model are integrated within the DRS system.

The decision-making layer evaluates the context and selects either the rule-based approach or the machine learning model to determine the relevant response segments.

### Testing and Optimization

The hybrid system is thoroughly tested to ensure seamless interaction between the rule-based and machine learning components.

### An Implementation Example

When a user says “take a picture”, the application captures an image and sends a request incorporating the image to the server. The DRS initially uses a basic rule to determine that the “brief commands” segment is relevant in the “real-time yoga session” state.

The server processes the image and returns a complete response with multiple segments.

The rule engine selects the “brief commands” segment based on the predefined rule and presents it to the user.

If the user performs a more complex action, such as navigating through multiple segments and frequently requesting additional information, the DRS switches to the machine learning model.

The machine learning model predicts the most relevant segments based on the user's behavior and the application's state, ensuring that the user receives contextually appropriate information.

The decision-making layer dynamically adjusts between rule-based logic and machine learning predictions, providing an optimized and responsive user experience.

### An Example Application Scenario

One of the application scenarios according to the technology disclosed herein involves an app configured, in a first operational state, to listen to an audio input for user commands. The audio input can be from the device the app is running on or external audio provided to the app.

The app can also be configured to use, in the first state, a speech recognition service, running locally or on a remote server or in the cloud, to recognize specific predefined user commands (e.g., specific words or phrases) in the audio input. The service can be part of the app itself.

Alternatively or additionally, the app or the service can recognize user commands or intent through audio analysis and inferring user intent and/or commands related to the app's functionality without relying on a fixed list of phrases or words.

The app or the service can use a pre-trained neural network, machine learning system, or an artificial intelligence (AI) agent for audio analysis or speech recognition.

In the context of this patent document, the term “AI” encompasses various types of neural networks and machine learning systems, software, and algorithms. For example, the AI agent can determine whether the user wants to take a picture using the phone's camera based on the audio provided to the agent.

When the app in the first state recognizes or receives a command from the app user such as “take a picture,” it can perform the following steps:

Image Capture: The app takes a picture or video using the camera of the device it is running on.

Server Request: The app includes the captured picture or video as part of a request to a server. The server can be a remote server or the same device the app is running on. The server can be part of the app itself.

Response Reception and Action: The server responds with one or multiple parts or segments in the response.

If the app is still in the first operational state when it receives the server response, it presents a first part of the response via text, audio, video, and/or images.

Upon receiving a “stop” or “more info” command from the user, the app transitions from the first state to a second state and presents a second part of the response, different from the one it presented in its first state.

If no server response is received in the first state, the app transitions to a third state instead of the second upon receiving the “stop” or “more info” command.

If the server response does not include a distinct first part or segment, the app may present the whole response or a special message.

### Example Embodiment

An embodiment of the disclosed technology can be implemented in an app with some or all of the following functionality:

### App Overview

A yoga app, according to the disclosed technology, helps users learn, practice, and perfect various yoga poses (asanas). It provides tools for comparing user poses with reference images, either by uploading photos from the gallery or using the camera in real time.

The device's camera is voice-controlled, so there is no need to hold the device to take a photo. When using the camera in “real-time” mode, the app gives brief voice commands to adjust the user's pose.

The user can stop the camera with a voice command, view more detailed instructions on correcting their pose, and compare their pose photo with the reference photo.

When working with photos from the device's gallery, the app provides a list of differences between the reference and user poses/asanas.

Users can control the number of pose correction commands and add their own poses, which do not necessarily have to be yoga poses.

The app includes a gamified or competitive element, where users aim to “hit the pose” so accurately that the AI cannot distinguish their pose from the reference.

Additionally, users can challenge each other to replicate (as judged by the AI) their “unusual” or “standard” poses.

The app supports video in addition to photos, which can be useful for both yoga practice and activities like dancing or ballet.

The main functionality is comparing one's poses (yoga poses and other poses, such as dance, ballet, or arbitrary poses) with reference images or videos to receive feedback and recommendations for adjustments.

An embodiment of the app allows users to compare a video clip (with an audio track in it) with a reference video clip of, e.g., the physical activity the user is trying to copy.

Therefore, the term “photo” or “image” in this patent document can be substituted with the term “video” (and vice versa) without any departure from the scope of the disclosed technology. Similarly, the term “pose” can be substituted with the term “sequence of movements” (and vice versa) anywhere in this patent document without any departure from the scope of the disclosed technology.

### Key Features and Interface Elements

Main Menu. See FIGS. 1 and 2.

Main Menu Button (Top Left Corner): Tapping this opens the main navigation menu, providing access to different sections of the app.

Profile Information: The top section of the menu displays the user's name and email.

Main Menu Sections. See FIG. 2.

Poses/Asanas: The default section for selecting a reference yoga pose and a pose for comparison.

Add a Pose: Navigate here to add new poses to the list.

Settings: Access and modify the app settings.

How To: View instructions and a user guide on how to use the app.

“Add a Pose” (also referred to as “Add-a-Pose” in this patent document) Section of the Main Menu. See FIG. 3.

Pose List: View a list of all saved reference poses that a user added to the app (the app includes a number of reference poses by default). Each pose entry includes a thumbnail image, the name of the pose, and options to edit or delete the pose.

Edit and Delete: Use the “EDIT” button to modify a pose's details or the “DELETE” button to remove it from the list.

Add Button (+): Tap this button to add a new pose to the list.

Poses/Asanas Screen. See FIG. 4.

Reference Pose Selection: Choose a reference pose from a dropdown menu to compare the user's pose against it. The reference pose can be a standard yoga pose, a ballet pose, a pose typical for any other type of physical activity or exercise, or an arbitrary pose.

‘Determine Yoga Pose from the Image’ Option: The dropdown menu also has this option, allowing the user to practice and receive feedback for different yoga poses without selecting a specific pose. When selected, the app instructs the AI agent to determine the yoga pose using the user's image.

Example Usage: The reference pose image can be a photo of the user's yoga pose taken by their yoga instructor after the instructor has set up and verified the pose.

Compare with a Photo from the Gallery/Photos: Upload a photo from the device's gallery to compare it with the selected reference pose.

Compare with Real-Time Camera: Use the device's camera to compare the current pose in real-time with the reference pose.

Settings Screen. See FIG. 5.

API Key Input: Users can enter their API key to enable some app functionalities.

Corrections Count: Set the number of corrections or adjustments the app should suggest.

Photo and Album Access. See FIG. 6.

Photos/Albums Selection: When uploading a photo after selecting “Compare with a Photo from the Gallery/Photos” on the “Poses/Asanas” screen, the app allows selecting images from recent photos or specific albums, including those from linked accounts like Google Photos or other cloud photo services.

Pose Comparison with Guidance when Working with Photos from the Gallery/Photos. See FIGS. 7 and 8.

Photo Selection: When the app user wants to compare a pose in a previously taken photo with a reference pose, they can click the “Compare with a Photo from the Gallery/Photos” button shown in FIG. 4 and select a photo from their device or online/cloud accounts.

### Server Request and Response

Request Formation: After the user selects a photo or video for comparison, the app sends a request to a server. The request can include the selected reference image or video retrieved from the model/pose store (170), the user's photo or video, and/or instructions for an AI software or agent running on the server.

Server Processing: The server analyzes the images using the AI software/agent to determine differences between the reference and user poses or sequences of movements.

Response Generation: Based on this analysis, the server creates a list of pose correction instructions designed to bring the user's pose closer to the reference pose. It also generates a list of shorter commands with essential actions required for pose correction. These lists are then returned to the app as different segments in the server's response.

Non-Deterministic AI Agent: The AI agent/software running on the server can be non-deterministic, meaning it can generate different responses when provided with the same inputs (e.g., input images).

Example Response: For the Reference Pose shown in FIG. 7 and the User Pose shown in FIG. 8, the pose correction instructions included:

“The pose alignment is close to the target. Bend the front knee more deeply to align it directly over the ankle. Lower the hips slightly to deepen the stance. Straighten the back leg more fully, pressing the heel into the ground. Extend the arms more actively, reaching through the fingertips. Rotate the torso slightly more to face the side”.

The shorter commands included:

“Bend the front knee more deeply.”

“Lower the hips slightly.”

“Straighten the back leg more fully.”

“Extend the arms more actively.”

“Rotate the torso slightly more.”

Corrections Count: The number of instructions (5) corresponded to the “Corrections Count” setting set to 5, as shown in FIG. 5. This parameter was part of the app request to the server.

### Reference Pose and Selected Pose Comparison

Side-by-Side Display: When a response from the server is received, because the user's image for comparison was selected from the photos previously saved in the device's Gallery/Photos, the app provides a side-by-side comparison of the reference pose and the user's pose, with alignment feedback and (optionally) improvement suggestions below.

Alignment Feedback: Includes (wholly or partially) the list of pose correction instructions or the list of shorter commands.

Dynamic Response Selection (DRS): Because the user has chosen a photo from the device's Gallery/Photos, the DRS within the app selects the list of pose correction instructions from the server's response as the alignment feedback and displays it under the images, as shown in FIGS. 7 and 8.

User Interaction: The user can swipe between the reference and their photo to visually compare them in the context of the feedback and suggestions.

Pose Comparison with Guidance when Working with Real-Time Camera. See FIGS. 9 and 10.

### Live Camera Comparison

Initiating Comparison: If the user wants to compare their current pose with a reference image, they select the reference pose from the dropdown menu in FIG. 4 and click the “Compare with Real-Time Camera” button.

Live Feed Display: The app transitions to a screen (FIG. 9) displaying a live feed from the device's camera. The user can set the device on a tripod or stable surface.

Camera Switch: Provides a camera switch icon or button to switch between different device cameras.

### Voice-Controlled Commands

Audio Signals: The app generates an audio signal at predetermined intervals (e.g., every 2-3 seconds), which can be, for example, set in the app's settings.

Listening Interval: After each signal, the app listens for a specified time (e.g., 2 seconds) for user commands.

Command Words/Phrases: Users can say a “code word” or “code phrase” (e.g., “ok”, “go”, “take a photo”) to trigger the app to take a picture or video.

Customizable Commands: These words and phrases can be, e.g., set by the user in the app or device's operating system.

Visual Indications: The app may use visual cues (e.g., blinking flashlight, screen changes) to indicate listening periods.

Gesture Commands: Users can also give commands via gestures; the app processes captured images or videos to detect these commands.

Continuous Monitoring: Alternatively, the app can continuously monitor for commands without audio or visual signals.

### Command Recognition

Audio Processing: The app records audio clips after signals and sends them to a speech-to-text service (local or cloud-based).

Speech-to-Text Conversion: Converts audio to text transcripts.

Keyword Matching: Uses algorithms like regular expressions, contextual filtering, threshold-based detection, and NLP methods to identify command words or phrases.

### Image Capture and Server Request

Triggering Capture: Recognizing a certain keyword or keyphrase prompts the app to capture a photo or video.

Processing and Sending Data: The app processes the media (e.g., resizing images, cropping/extracting the user-containing part of the image, etc.) and sends it to the server, including the reference image and instructions for an AI agent (Note: a reference image can be omitted if the user selects ‘Determine Yoga Pose from the Image’ from the dropdown menu on the Poses/Asanas screen).

Pose Identification: If no reference image is included, the AI agent identifies the pose or movements the user is attempting using the image or video of the user. In this case, because no reference image or video was provided to the AI agent, the user's pose or movements will be compared, by the AI agent, with the (“ideal”) version of the identified pose or movements that the AI agent learned previously or which the AI agent has knowledge or information about. In some implementations, the AI agent instructions can include the name of the yoga pose or the movement sequence the user is attempting. If the request to the server includes a reference image, the pose which the user is attempting to reproduce can be identified by the AI agent using the reference image. In that case, the AI agent will use the provided reference image to generate feedback for the user.

### Post-request Actions

Resuming Command Listening: After sending the request, the app continues to listen for commands.

User Notification: Indicates to the user that a server request is sent and a response is pending.

### Server Response and Dynamic Selection

Request Processing and Response Generation: A machine learning system on the server processes the request and generates a response with multiple segments. The application can instruct the system generating the server response on how to separate and/or format the response sections. The server response can include text, audio, video, or a combination of these and other media.

Short Instructions (or Commands): One segment includes short instructions on correcting the user's pose or movements to align with the reference pose or “ideal” pose.

Text-to-Speech (TTS): The app may vocalize these short instructions using a TTS component.

User-Controlled Length: Verbosity of the instructions and their number can be user-defined to, e.g., minimize exercise interruption.

TTS Options: TTS can be part of the app, a separate application, or run on a server.

Audio Segments: The response may include audio files of the (short and/or detailed) instructions.

Detailed Instructions: Another segment provides more comprehensive guidance, possibly with text, images, audio, or video, explaining differences and corrections.

Additional Content: May include generated images or videos of the ideal pose and/or instructions from a default position.

### Dynamic Response Selector (DRS)

Response Selection: After receiving the server response, the DRS selects which segment to present based on current or prior app states and user actions.

User Engagement: If the user remains engaged in the current app state (Live Camera Comparison), the app presents the short instructions, possibly vocalized.

Interruptions: Users can interrupt the TTS by swiping or issuing commands.

Resuming Interaction: Post-instruction, the app resumes signaling and listening for commands.

Response Management: If the user navigates away before the response is ready, the app may discard or archive the response.

### Accessing Detailed Instructions

Stopping Command Loop: Users can say “Stop” to exit the command listening mode.

Transitioning Screens: The app then shows a screen (like FIGS. 7 and 8) where detailed instructions are available.

Saving Data: Users can save information, including images, to local or remote storage (e.g., device's Gallery or Photos).

The application can be configured to instruct the user to change their position relative to the camera to optimize image capture for analysis. If the application detects (by itself or using the AI agent, for example) that the user is too close or too far from the camera, or not fully within the frame, it can provide visual or auditory prompts such as “Please step back slightly”, “Move a bit to your right”, or “Ensure your entire body is visible on the screen”. Additionally, the client-side application can automatically switch among the device's cameras (such as, e.g., front-facing, rear-facing, or wide-angle lenses) to adjust the field of view and ensure that the user's entire body is appropriately captured. This automatic camera switching can be based, e.g., on the application's assessment of the user's position and the current camera's suitability. By guiding the user on positioning and dynamically selecting the optimal camera, the application captures high-quality visual data, enhancing the accuracy of pose detection and feedback from the AI agent. The application may utilize computer vision algorithms on the client side to assess the user's positioning relative to the camera and generate appropriate instructions or perform automatic adjustments. This feature enhances the user experience by facilitating optimal camera framing without requiring manual adjustments or trial-and-error by the user.

### Example of a Server Request and a Server Response with Multiple Segments for the Yoga Application

### Server Request

Endpoint: POST/analyze-pose

### Headers

Content-Type: application/json

Authorization: Bearer <user-access-token>

Body: The client sends a request to the server with the following JSON content:

userId: “123456”

timestamp: “2024-07-08T12:00:00Z”

poseImage: “<base64-encoded-image-data>”

referencePoseImage: “<base64-encoded-image-data>”

### deviceInfo

deviceId: “abcdef123456”

deviceType: “mobile”

osVersion: “Android 12”

requestedSegments: [“identification”, “corrections”, “commands”, “tips”, “products”]

### Server Response

Status Code: 200 OK

### Headers

Content-Type: application/json

Body: The server responds with a JSON object that includes the user ID, timestamp, and multiple segments:

### Identification Segment

poseName: “Warrior II”

poseId: “pose_001”

description: “Warrior II (Virabhadrasana II) is a standing yoga pose that enhances strength, stability, and concentration.”

### Corrections Segment

Align your front knee over your ankle to ensure proper support.

Lower your hips slightly to deepen the stance and improve balance.

Straighten your back leg more fully and press your heel into the ground to stabilize the pose.

Extend your arms actively, reaching through the fingertips to engage the shoulders.

Rotate your torso slightly more to face the side, opening up your chest.

### Commands Segment

Align knee over ankle.

Lower hips.

Straighten back leg.

Extend arms.

Rotate Torso to the Side.

### Tips Segment

breathing: “Inhale as you raise your arms and exhale as you bend your front knee.”

focus: “Gaze over your front hand and keep your shoulders relaxed.”

Products Segment (e.g., composed by the AI agent using the reference image provided in the request):

productId: “prod_001”

productName: “Yoga Mat”

productDescription: “Non-slip yoga mat for enhanced stability.”

productUrl: “https://example.com/yoga-mat”

productId: “prod_002”

productName: “Yoga Blocks”

productDescription: “Foam blocks for support in various poses.”

productUrl: “https://example.com/yoga-blocks”

### Explanation

### Server Request

The client sends a POST request to the/analyze-pose endpoint with headers specifying the content type as JSON and including an authorization token. The body of the request contains the user ID, timestamp, base64-encoded user pose image, base64-encoded reference pose image, device information, and a list of requested response segments.

### Server Response

The server responds with a 200 OK status and a JSON object. This object includes:

Identification Segment: Provides details such as the name, ID, and description of the identified yoga pose (Warrior II).

Corrections Segment: Offers detailed corrections to improve the user's pose alignment.

Commands Segment: Provides concise commands derived from the corrections for quick reference or audio feedback during the practice.

Tips Segment: Gives additional advice on breathing and focus to enhance practice.

Products Segment: Suggests related products, like a yoga mat and yoga blocks, with descriptions and purchase URLs.

### Some Advantages of the Disclosed Technology

Improved User Experience: The disclosed technology significantly enhances the user experience by providing responsive and contextually appropriate content and actions. By dynamically selecting and presenting information based on user interactions and current app states, the application ensures that users receive the most relevant and useful information without unnecessary steps.

Increased Efficiency: The technology reduces unnecessary processing by handling only the relevant segments of server responses on user devices. By focusing on delivering specific information pertinent to the user's current context and actions, the application avoids processing and displaying extraneous data. This targeted approach conserves device resources and streamlines the app's operations.

Enhanced Performance: The application's performance is improved by minimizing data handling and processing overhead. By selectively processing and presenting server response segments, the app reduces the amount of data that needs to be transmitted, stored, and managed on the device. This optimization leads to faster response times, reduced latency, and a smoother overall user experience.

### Client-Side Splitting and/or Modification of AI Agent Responses

In some implementations, the client-side application can be configured to perform additional splitting or modification of the AI agent's response, providing an alternative to relying solely on pre-segmented responses from the AI agent. This approach can be beneficial in scenarios where the AI agent returns a single, comprehensive response or when further customization of the feedback is desired to better suit the user's context or preferences.

The client-side application can be programmed or configured to analyze the AI agent's response and dynamically segment it into multiple parts based on specific criteria. For example, the application can parse the response to identify different types of information such as safety-critical instructions, performance enhancements, motivational messages, or commercial content. By doing so, the application can tailor the presentation of feedback to the user's immediate needs and the application's current state.

Even when the AI agent's responses already contain multiple segments, the client-side application can further split these segments or reorganize the information to provide a more granular or context-specific delivery of feedback. This can be achieved by applying, e.g., natural language processing (NLP) techniques, keyword spotting, or pattern recognition algorithms to dissect the response into finer components.

### Advantages and Purposes of Client-Side Splitting

One advantage of client-side splitting is the ability to customize feedback in real time without requiring changes to the AI agent's processing or response format. This flexibility allows the application to adapt to various user preferences, languages, or accessibility requirements. For instance, the application can be configured to prioritize safety instructions for novice users while providing advanced technical feedback to experienced users. The application can, e.g., translate the English language AI agent response into another language (e.g., the one selected by the user in the app or the current language of the user's operating system).

Client-side modification can also enhance privacy and compliance with data handling regulations. By controlling how and when certain information is presented, the application can ensure that sensitive data is handled appropriately and that the user is not exposed to unwanted content.

Additionally, performing splitting on the client side can reduce dependency on the AI agent's capabilities, enabling the application to function effectively even with AI agents that provide unsegmented or differently structured responses. This can be particularly useful when integrating third-party AI services over which the application developer has limited control.

### Example Criteria for Splitting and Modification

The criteria for splitting the AI agent's response on the client side can include (Note: these criteria can also be applied to splitting the AI agent's response on the AI agent's side (e.g., by the AI agent itself)):

Content Type: Distinguishing between textual instructions, visual aids, or audio cues to present them through the most appropriate medium.

Urgency and Priority: Identifying safety-critical feedback that needs immediate attention versus informational content that can be reviewed later.

User Context: Considering the user's current activity, skill level, or expressed preferences to tailor the feedback accordingly.

Language and Localization: Detecting and translating segments into the user's preferred language or adjusting terminology to match regional dialects.

Compliance and Filtering: Removing or modifying content that does not meet regulatory standards or the application's content policies.

### Example Implementation Strategies

The client-side application can be implemented to use NLP libraries and parsing techniques to analyze the AI agent's response. For example, the application can be configured to:

Use Regular Expressions: Identify specific phrases or keywords indicating different types of feedback, such as “Warning”, “Tip”, “Next Step”, or “Commercial Offer”.

Apply Sentiment Analysis: Detect the tone of the feedback to adjust motivational messages or filter out negative language.

Implement Topic Modeling: Group related pieces of information together, making it easier to present coherent segments to the user.

Leverage Machine Learning Models: Train models to recognize and classify different parts of the response based on historical data and user interactions.

### Results and Utilization

The results of client-side splitting and/or modification can lead to a more personalized and efficient user experience (Note: the same applies to the AI agent-side response splitting and modification). By controlling the granularity and sequence of feedback presentation, the application can:

Enhance Real-Time Guidance: Provide immediate, actionable feedback during exercise without overwhelming the user with too much information.

Improve Learning Outcomes: Allow users to delve deeper into detailed instructions at their own pace, facilitating better understanding and retention.

Optimize User Engagement: Present motivational content or achievements in a way that encourages continued use of the application.

Facilitate Accessibility: Adapt the format and/or complexity of feedback to meet the needs of users with disabilities or differing levels of expertise.

### Potential Benefits of the Client-Side Splitting and/or Modification

While multi-segment AI agent responses offer structured feedback, client-side splitting and/or modification can provide some additional benefits in certain embodiments:

Greater Control: Developers can fine-tune how feedback is categorized and delivered without modifying the AI agent.

Adaptability: The application can adjust to changes in the AI agent's response format or content without requiring extensive updates.

Customization: The application can implement user-specific rules or preferences for feedback that the AI agent may not support.

Implementing client-side splitting and/or modification of AI agent responses can enhance the flexibility and effectiveness of the feedback provided to the user in some scenarios. By tailoring the feedback to the user's context and preferences, the application can improve the overall user experience, promote better engagement, and ensure that the guidance offered is both relevant and actionable.

### Interactive Feedback and Follow-Up Questions

The disclosed technology further enhances user engagement and learning by allowing the user to interact with the AI agent through personalized questions about their pose sequence execution shown in a video. The user can include specific questions as part of the initial request sent to the AI agent along with the video data. For example, the user might ask, “Am I keeping my back straight during the downward-facing dog pose?” or “How can I improve my balance in the tree pose?” These questions can be input via voice commands, typed text, or selected from a list of suggested inquiries within the application.

Upon receiving the request, the AI agent can process both the video of the user's performance and the accompanying question(s) and generate a response that directly addresses the user's concerns. The AI agent can utilize natural language processing to understand the question and apply computer vision techniques to analyze the user's movements in the video. This allows the AI agent to provide precise, actionable feedback tailored to the user's specific inquiries.

After reviewing the feedback from the AI agent, the user can ask follow-up questions to gain deeper insights or clarification. For instance, if the AI agent suggests adjusting foot placement to improve stability, the user might ask, “Can you show me the correct foot position?” or “What exercises can help strengthen my ankles?” The client-side application can facilitate this iterative interaction by, e.g., capturing the user's follow-up questions and forming additional requests to the AI agent. This conversational exchange can continue, enabling a dynamic and personalized coaching experience.

Implementing this feature can involve, e. g, integrating a conversational interface within the client-side application. The application can manage the dialogue context, ensuring that each follow-up question and response is coherent and relevant to the previous interaction. The AI agent can be configured to handle multi-turn conversations, maintaining awareness of the discussion's history to provide comprehensive assistance.

This interactive feedback mechanism offers several advantages:

Enhanced Personalization: Users receive guidance tailored to their specific questions and concerns, which can lead to more effective learning and improvement.

Deeper Understanding: Engaging in a dialogue allows users to explore the nuances of their performance, leading to a better grasp of techniques and principles.

Increased Engagement: The ability to ask questions and receive immediate answers creates a more engaging and motivating experience, encouraging active participation in the learning process.

Adaptive Learning: The AI agent can adjust its feedback based on the user's inquiries, providing customized recommendations and progressions suited to the user's skill level.

Flexibility: Users can utilize this feature with both images and videos, making it adaptable to different practice scenarios and preferences.

The client-side application can be configured to present this interactive feature through various user interface elements, such as, e.g., a chat window, voice interaction module, or dedicated Q&A section. The application ensures that the user's questions and the AI agent's responses are seamlessly integrated into the user experience, allowing for uninterrupted practice and learning.

Additionally, the application or the AI agent can store the history of interactions, enabling users to revisit previous questions and feedback. This archive can serve as a valuable resource for tracking progress over time and reinforcing learning outcomes.

In some implementations, to optimize performance and responsiveness, initial processing of the user's questions can occur on the client device, determining, e.g., intent and relevance before forming the request to the AI agent. This approach leverages edge computing to reduce latency and enhance the immediacy of the interaction.

Overall, enabling users to ask questions about their pose sequence execution and receive detailed, structured and personalized feedback from the AI agent significantly enriches the functionality of the virtual fitness trainer. It transforms the application from a one-way feedback tool into an interactive coaching platform, fostering a more engaging and effective learning environment.

### Extension to Image-Based Queries

In addition to video analysis, the disclosed technology allows users to ask questions about static images depicting their poses. E.g., the user can capture or select an image of themselves performing a pose and include specific questions as part of the request to the AI agent. For example, the user may ask, “What am I doing correctly and what am I doing incorrectly in this pose?” This enables the AI agent to provide detailed feedback on both the strengths and areas for improvement within the user's form as depicted in the image.

The AI agent can analyze the image using, e.g., computer vision techniques to assess the user's alignment, posture, and adherence to the correct form of the pose. It then generates a response that highlights the aspects the user is executing correctly (e.g., in one segment of the response), reinforcing positive behaviors, and identifies mistakes or deviations that need correction (e.g., in another segment of the response). This dual feedback approach helps the user understand not only where improvements are needed but also recognizes their successes, enhancing motivation and confidence.

Similarly, when dealing with movement sequences shown in videos, the user may ask, “What am I doing right and wrong in this movement sequence?” The AI agent can evaluate the entire sequence, providing feedback on the flow, timing, and execution of specific movements (in, e.g., different segments of its response). By pinpointing specific moments where the user excels or requires adjustment, the AI agent offers comprehensive guidance that covers both individual poses and transitional movements.

Implementing this feature can involve the client-side application facilitating the inclusion of questions about images in the requests sent to the AI agent. The application can provide interface elements such as text input fields or voice recognition capabilities to capture the user's inquiries. The AI agent can be configured to process these questions in conjunction with the visual data, ensuring that the feedback is specific and relevant to the user's concerns.

The ability to ask questions about images and videos expands the application's interactive capabilities, making it a versatile tool for users to gain in-depth understanding of their practice. It fosters a supportive learning environment where users receive constructive feedback that guides them toward achieving their fitness goals.

### Voice-Based User Instructions and AI Agent Interaction

In some embodiments, the users can give instructions and commands to the AI agent via voice or speech inputs. The client-side application can incorporate or use external voice recognition capabilities (e.g., OpenAI's Whisper), allowing users to interact with the system naturally and hands-free. For example, during a workout session, the user can say, “Analyze my last pose”, or “Provide feedback on my balance”, and the application will capture the audio input, process it using the voice recognition capabilities to determine the user's intent, and form a corresponding request to the AI agent. This voice-based interaction enhances user convenience, especially during physical activities where manual interaction with the device may not be practical.

The AI agent can be an omni-modal or multimodal Large Language Model (LLM) capable of accepting and processing multiple types of input data, including images, videos, audio, and text. This omni-LLM can be configured to interpret and understand complex, multimodal inputs, allowing it to analyze visual data in conjunction with textual or spoken instructions provided by the user. The AI agent's ability to process diverse data types enables it to follow user instructions effectively and generate comprehensive, context-aware feedback. For instance, if a user says, “Watch my sequence and tell me where I can improve”, while uploading a video of their exercise routine, the AI agent can analyze the video in light of the user's request and provide targeted advice.

In generating responses, the AI agent can include audio, image, or video elements that vary in detail and/or length. The AI agent's feedback can be structured to contain both brief, concise guidance suitable for immediate action and more detailed, in-depth explanations for thorough understanding. For example, the response may include a short audio clip with quick correction tips for use during the exercise and a longer video demonstrating the correct form for the user to review afterward. The inclusion of both brief and detailed versions in the same response allows the client-side application to select and present the most appropriate content based on the current application state and user preferences, as managed by the Dynamic Response Selector (DRS).

The client-side application or the AI agent can guide the user on how to perform the pose corrections from the list of corrections returned by the AI agent. This guidance can be delivered, e.g., through step-by-step instructions, visual demonstrations, or interactive tutorials. For example, if the AI agent identifies that the user's knee alignment needs adjustment in a yoga pose, it can provide a detailed explanation of the correct alignment, accompanied by annotated images or a video demonstration. The application can facilitate a dialogue with the user, where the user asks follow-up questions like, “Can you show me how to adjust my knee position?” and the AI agent responds with tailored guidance.

This interactive guidance extends to other forms of feedback provided by the AI agent. Whether the user seeks advice on breathing techniques, balance improvement, or transition movements between poses, the AI agent can offer comprehensive assistance. By leveraging the dialogue feature described above, the user can engage in a conversational exchange with the AI agent, receiving personalized coaching that adapts to their needs and learning pace. The client-side application manages this interaction seamlessly, ensuring that the user can focus on their practice while accessing the support they require.

### Continuous-Streaming Embodiment

In some embodiments, the client device (110) establishes a persistent, full-duplex session with the AI agent (150) over encrypted transport (500) and continuously streams video frames from camera(s) (120/121-126) and audio from the microphone (130), optionally accompanied by wearable summaries (180). Unlike request/response batches (FIG. 19), this mode maintains a live stream so the agent can produce incremental (“rolling”) multi-segment outputs that the client's DRS (250/selection logic 474) consumes as they arrive. The architecture remains that of FIG. 11-14 and 20-23/28; only the transport cadence and scheduling semantics change to support real-time guidance with lower round-trip overhead.

Client capture, pre-processing, and stream formation. The capture module (210) acquires continuous RGB (121) and, when available, depth/IR/ToF/LiDAR/thermal (122-126) plus audio. The pre-processing module (220) performs privacy-first steps before egress (e.g., subject-region cropping, face redaction/anonymization (485), exposure/framing normalization, and optional enhanced-video layers) then packetizes frames/audio into a time-stamped stream tagged with pseudonymous session IDs (488). Adaptive policies modulate resolution, frame-rate, and keyframe cadence to track bandwidth/thermal budgets (118, 119/160), while quality/context flags (620) ride alongside to inform both the agent and the DRS. Where policy applies, the client does not compute keypoints and streams redacted pixels/sidecar features only.

Transport and session control. The streaming session runs over encrypted transport (500) with keep-alive/heartbeat, sequence numbers, and back-pressure handling: if uplink degrades, the client drops to lower bitrate profiles (e.g., fewer auxiliary channels, reduced fps) and prioritizes user-region crops; if loss persists, the client falls back to brief-only local guidance and cached assets (486) until recovery. All artifacts (on-device and server-side caches) obey retention/delete controls (487) and pseudonymous association (488). These controls align with FIG. 17-18 and the network topologies of FIG. 28 (direct or intermediated).

Agent-side streaming pipeline and rolling bundles. The agent (150) ingests the live stream (145) and processes sliding windows through the FIG. 13 stack (pose estimation (151) (explicit or keypoint-optional), movement/phase analysis (152, 610-613), breathing analysis (153, 614), and severity classification (154, 615)). The feedback generator (156) emits incremental deltas (e.g., “next brief cue”, “updated timing offset”, “overlay directive changes”), and the response segmenter (155) assembles them into rolling multi-segment bundles (471 brief, 472 detailed, 473 additional) that carry validity windows and phase IDs so the client can safely preempt or coalesce guidance near boundaries. This avoids re-requesting content on every turnover and keeps selection deterministic under tight deadlines.

DRS operation under continuous streaming. Upon each response-arrival trigger (600), the client's DRS (250) reads time-to-boundary (B), severity (S), quality/context (Q/620), connectivity (C), and app state (A) (e.g., practice, hold/review, idle/explore) and applies the priority ladder: (i) imminent/safety→brief only (≤corrections-count 32) with optional TTS; (ii) holds/review→elevate detailed with overlays; (iii) explore/idle→surface additional information while suppressing disruptive cues. The DRS enforces hysteresis near boundaries to prevent oscillation, coalesces repeats (e.g., “repeat last cue”), and annotates HUD (FIG. 10) with the currently active brief plus an expand affordance to open its matching detailed explanation when safe. Selection is deadline-driven: items expiring before the next boundary are either spoken immediately or dropped; deferred items are cached for the next viable window. See FIGS. 14 and 20.

Audio streaming: breathing+commands. Audio is streamed continuously (or in listening windows) to support breathing inference (153/614) and hands-free control (voice intents). The DRS treats recognized intents (repeat, more detail, pause, next tip) as state inputs and applies debounce/coalescing so frequent requests do not thrash TTS or overlays. When the agent's breath-sync signal indicates late/early phases, the DRS injects a breathing resynchronization brief ahead of the next egress/ingress and defers non-critical detail.

Quality loop, fusion, and camera/angle control while streaming. If quality/context (620) flags low light, occlusion, narrow FOV, or low confidence, the client executes the 620→621→622→623 loop of FIG. 22 without breaking the stream: request a framing or lighting adjustment; switch cameras (front/rear/wide) or hand off to an external/drone camera (195); pull a brief auxiliary depth/IR burst; or integrate wearable summaries (180). The fusion module (127) aligns these inputs and updates the stream with improved features so the agent can return higher-confidence bundles; the DRS may temporarily substitute camera/lighting tips for nuanced form cues until confidence recovers. See FIGS. 16, 22, and 27.

Privacy, caching, partition, and failover. All outbound media pass through on-device redaction/anonymization (485) prior to encryption (500); compression/caching (486) minimize bytes on the wire and enable brief-only continuity if the stream stalls. Partition policy (FIG. 17) can keep TTS/overlays local while the agent performs heavy analysis; if connectivity drops, the client falls back to local cached briefs, visual alignment guides, or a local-only pipeline (481) where provisioned, then resumes the stream and reconciles timing when the link recovers. Provenance, retention (487), and pseudonymous IDs (488) remain in effect throughout (FIG. 23).

Illustrative run-through (streaming Vinyasa). The user starts Live Comparison (FIG. 4); the client opens the stream and begins sending redacted video/audio. The agent detects an approaching ingress→hold boundary (611) and emits a brief (“step the right foot forward”), plus an overlay directive for a body-anchored arrow (360). The DRS, seeing B≈300 ms and S=moderate, speaks the brief now and draws only the arrow. On hold, the agent pushes a richer detailed item describing knee-over-ankle and pelvis cues; the DRS expands it alongside the live feed or in the review panel depending on A. When low light triggers 620, the client requests torch/on-tripod guidance and switches to a wider lens; fusion improves confidence and the agent upsamples cadence advice on the next window without tearing down the stream.

Incorporating these features enhances the overall functionality of the virtual fitness trainer, making it a versatile and adaptive tool for users of varying skill levels. The ability to give voice instructions, receive multimodal feedback, and engage in guided corrections empowers users to achieve better outcomes in their physical exercises. It also exemplifies the integration of advanced AI capabilities within the client-side application to deliver a highly personalized and effective training experience.

### Dynamic Instruction Storage on Non-Transitory Media

The disclosed technology can be implemented using a non-transitory computer-readable storage medium that, at different times, stores different sets of instructions which collectively provide the functionality according to the methods described herein. In some implementations, the non-transitory computer-readable storage medium is configured to store, at different times, different sets of instructions which collectively provide the functionality according to a method described in this patent document. The disclosed technology can also be implemented using a non-transitory computer-readable storage medium, which at different times is configured to store different sets of instructions that collectively provide the functionality according to a method described herein. This approach allows the client-side application to dynamically update, modify, or extend its capabilities by loading and storing various instruction modules as needed.

In practice, the client-side application can be designed with a modular architecture where specific features or functionalities are encapsulated within separate instruction sets or modules. These modules can be downloaded, updated, or removed from the storage medium based on factors such as user preferences, interactions, or (periodic) updates provided by the server.

For example, when a user decides to practice a new type of physical activity supported by the application, such as Pilates or dance, the application can retrieve the necessary instruction modules related to pose analysis, feedback generation, and interactive controls specific to that activity. These modules are then stored on the non-transitory storage medium of the user's device. Conversely, if the user no longer engages with a particular activity, the application can remove the corresponding instructions to free up storage space.

Over time, the non-transitory storage medium holds different instruction sets at different times, depending on the user's engagement and the application's operational context. This dynamic storage strategy ensures that the application remains adaptable and efficient, providing only the necessary functionalities when required. The cumulative effect of these varying instruction sets stored at different times is that the application, as a whole, maintains the full range of capabilities supported by the disclosed technology.

### Example Implementation Strategies

Module Management System: The application includes a module management component responsible for handling the downloading, updating, and removal of instruction sets. This system tracks which modules are currently stored on the device and manages dependencies between different instruction sets.

On-Demand Loading: Instruction sets are loaded onto the storage medium when, e.g., the user initiates a feature that requires them. For instance, if the user starts a session that involves advanced pose correction techniques, the application downloads the necessary modules to perform those functions.

Updates: The application periodically or on-demand checks for updates to the instruction sets stored on the device. Updated instructions can enhance performance, provide new features, or improve security.

User Preferences: Users can specify preferences that influence which instruction sets are stored. For example, they might choose to keep certain features always available offline, prompting the application to retain those instructions persistently.

### Example Advantages of Dynamic Instruction Storage

Resource Optimization: By storing only the necessary instruction sets at any given time, the application conserves device storage space and memory usage, leading to improved performance, especially on devices with limited resources.

Flexibility and Scalability: The modular approach allows the application to scale its functionalities according to user needs without requiring a full application update or reinstallation.

Personalization: Users receive a tailored experience as the application adapts to their specific activities and preferences, loading only the relevant instruction sets.

Security and Compliance: Storing instructions dynamically allows for timely updates and patches to be applied to specific modules, enhancing the overall security of the application.

### Example Scenario

Suppose a user primarily uses the application for yoga practice but decides to try a new feature involving dance movement analysis. When the user selects this new feature, the application downloads the corresponding instruction sets needed for dance movement recognition and feedback. These instructions are stored on the non-transitory storage medium and integrated into the application's operation. If the user later decides to focus solely on yoga again, the application can remove the dance-related instructions, thus optimizing storage space.

### Technical Considerations

Secure Transmission: When downloading new instruction sets, the application uses secure transmission protocols (e.g., HTTPS/TLS) and verifies the integrity and authenticity of the instructions through digital signatures or checksums.

Compatibility Management: The module management system handles compatibility between different instruction sets and the core application to prevent conflicts or errors.

Persistent Storage: Certain critical instruction sets required for the application's basic operation are persistently stored on the non-transitory medium, while optional or supplemental modules are managed dynamically.

By implementing a non-transitory storage medium that at different times stores different instructions, the disclosed technology achieves a balance between comprehensive functionality and efficient resource utilization. This dynamic storage approach enhances the application's adaptability and ensures that users have access to the features they need when they need them, without unnecessary overhead.

### System Implementation With Intermediary Server

In some implementations, a system according to the disclosed technology can be implemented using a client device, such as a phone or tablet, in communication with an intermediary server that is distinct from the server hosting the AI agent. This intermediary server can, for example, act as a conduit between the client device and the AI agent-hosting server, facilitating secure and efficient communication.

One of the primary functions of the intermediary server can be to manage sensitive information, such as API keys or authentication tokens required to access the AI agent's services. By storing and inserting these sensitive credentials into requests on behalf of the client device, the intermediary server enhances security by preventing exposure of such information on the client device, which may be less secure.

Additionally, the intermediary server can perform various processing tasks to optimize system performance and functionality. For example, it can handle request formatting, data preprocessing, or response parsing before transmitting data to the client device. It may also implement caching mechanisms to store recent responses from the AI agent, reducing latency and network load for repeated or similar requests.

The intermediary server can enforce policies and manage user authentication and authorization, ensuring that only permitted users or client devices can access the AI agent's services. It can also provide logging and monitoring capabilities, tracking usage patterns and detecting anomalies or unauthorized access attempts.

In such a system, the client device and the intermediary server collaborate to implement methods according to the disclosed technology. For example: The client device captures user data, such as images, videos, or voice commands, and generates requests that are transmitted to the intermediary server. The server then augments these requests with necessary credentials, performs any required preprocessing (including, e.g., image preprocessing in some embodiments), and forwards them to the AI agent-hosting server.

Upon receiving responses from the AI agent, the intermediary server can process or segment the responses if needed, potentially applying additional business logic or compliance checks. It then transmits the processed responses to the client device, where the Dynamic Response Selector (DRS) mechanism selects relevant segments based on the application state and/or user actions.

This architecture enhances the overall security, scalability, and maintainability of the system. By centralizing sensitive operations and credentials on the intermediary server, the system minimizes security risks associated with storing such information on client devices. It also allows for easier updates and maintenance of credentials and policies without requiring changes to the client-side application.

Furthermore, the intermediary server can support multiple client devices simultaneously, managing load balancing and optimizing resource utilization. This enables the system to scale effectively as the number of users increases, ensuring consistent performance and reliability of the services provided by the disclosed technology.

### Exclusion of Client-Side Pose Estimation and Skeletal Tracking

In certain embodiments of the disclosed technology, the client-side application and/or the intermediary server are designed or configured to refrain from detecting or determining key points (also referred to as “keypoints” in this document), performing skeletal tracking, or executing pose estimation algorithms. These components do not perform and are not configured to perform actions such as identifying skeletal joints, mapping the user's body to a skeletal model, or analyzing body posture and alignment on the client device or intermediary server. Instead, the images or videos (e.g., raw or preprocessed images or videos) captured by the client device are transmitted to the AI agent-hosting server without such processing.

In some embodiments of the disclosed technology, the client-side application and/or the intermediary server are explicitly configured not to detect or determine key points, perform skeletal tracking, or execute pose estimation algorithms. In some embodiments of the disclosed technology, the client-side application and/or the intermediary server are not configured to detect or determine key points, perform skeletal tracking, or execute pose estimation algorithms. As a consequence of that, these components do not perform actions such as identifying skeletal joints, extracting body landmarks, mapping the user's body to a skeletal model, or analyzing body posture and alignment on the client device or intermediary server. Instead, the images or videos captured by the client device are transmitted to the AI agent-hosting server without such processing.

### Integration of Robotic Devices

The disclosed technology incorporates various kinds of robotic devices to enhance the user's experience and effectiveness of physical exercise training. These robotic devices can, e.g., interact with the user and the client-side application to, e.g., provide real-time assistance, guidance, and feedback during exercise sessions.

### Robotic Assistance for Physical Guidance

In some implementations, robotic devices such as robotic arms, exoskeletons, or wearable robotic suits can be used to physically guide the user through poses or movements. The client-side application or the AI agent can communicate with the robotic device to control its movements based on the feedback generated by the AI agent. For example, if the AI agent detects that the user's posture needs adjustment, the robotic device can gently reposition the user's limbs to achieve the correct alignment. This physical guidance can help users, especially beginners or individuals with limited mobility, to learn proper techniques and reduce the risk of injury.

### Demonstration of Correct Movements

Robotic devices can also serve as demonstrators of correct poses or movement sequences. Humanoid robots or robotic mannequins equipped with articulating joints can mimic the correct execution of exercises as instructed by the AI agent. The client-side application or the AI agent can send commands to the robotic device to perform specific poses, allowing the user to observe the movements from different angles and better understand the proper form. This visual demonstration can complement the feedback provided by the application, enhancing the user's learning experience.

### Haptic Feedback and Sensory Cues

Wearable robotic devices or accessories, such as, e.g., haptic gloves or vests, can provide sensory feedback to the user in response to the AI agent's analysis. For instance, if the user's posture deviates from the correct alignment, the wearable device can generate tactile cues, such as, e.g., vibrations or gentle pressure, at specific body points to indicate where adjustments are needed. The client-side application can interpret the AI agent's feedback and translate it into haptic signals delivered by the robotic device. This real-time sensory feedback can help users make immediate corrections without diverting attention to visual or auditory prompts.

### Interactive Robotic Trainers

The system can incorporate interactive robotic trainers that engage with the user during exercise sessions. These robotic devices can communicate verbally, visually (by, e.g., showing images or videos on their screen(s) or flashing LEDs), or through gestures, providing encouragement, counting repetitions, or adjusting the difficulty level based on the user's performance. In some embodiments, the client-side application can coordinate the interaction between the AI agent and the robotic trainer, ensuring that the robot's responses are synchronized with the user's actions and the feedback provided by the AI agent. The camera and computing device of a system according to the disclosed technology can be incorporated, physically or communicatively, into such a robotic trainer. In some embodiments, the AI agent can be incorporated, physically or communicatively, into such a robotic trainer. This creates a more engaging and personalized training environment.

### Integration with Home Automation Systems

Devices integrated into home automation systems can enhance or change or adjust the exercise environment. For example, robotic assistants or such devices can adjust lighting, temperature, or music based on the user's preferences or the type of exercise being performed. The client-side application can send commands to these devices in response to user actions or AI agent feedback, creating an optimal setting for the user's workout session.

### Data Collection and Analysis

Robotic or other types of devices equipped with sensors can collect additional data about the user's movements, such as force exerted, range of motion, or muscle activation patterns. This data can be transmitted to the client-side application and forwarded to the AI agent for more comprehensive analysis. The AI agent can utilize this information to provide more precise feedback and recommendations.

### Remote Training and Telepresence

In scenarios where the user is training remotely, robotic devices can act as proxies for instructors or coaches. For example, a robotic device controlled by a remote trainer can demonstrate exercises or provide physical adjustments. The client-side application facilitates communication between the remote trainer and the robotic device, allowing real-time interaction and guidance.

### Safety and Accessibility Enhancements

For users with disabilities or special needs, robotic devices can provide assistance to make exercises more accessible. The client-side application can customize the interaction with the robotic device based on the user's capabilities and the AI agent's recommendations.

The client-side application can support communication protocols and interfaces compatible with the robotic hardware. This may involve using standard communication technologies such as Bluetooth, Wi-Fi, or specialized APIs provided by the robotic device manufacturers. The application can be configured to manage the synchronization between the user's actions, the AI agent's feedback, and the robotic device's responses to ensure a seamless experience.

Some embodiments of the disclosed technology include a method and system for providing real-time feedback to users performing physical exercises via a client-side application on a computing device. The application captures images or videos of the user using the device's camera(s) and may automatically switch between cameras or instruct the user to adjust their position relative to the camera to optimize image capture. The captured data is sent to a server hosting an AI agent capable of processing multimodal inputs, including images, videos, and text or voice instructions from the user. The AI agent analyzes the user's performance and generates a response comprising multiple segments with feedback at varying levels of detail, which may include brief and detailed audio, visual, or textual elements. A dynamic response selector within the application determines the current state and/or user actions to select relevant response segments for presentation. The application supports interactive dialogs with the AI agent, allowing users to ask questions about their performance and receive personalized guidance. It can operate in offline mode using pre-downloaded models to provide real-time feedback without server communication.

### Example Embodiments Using Quantum Computing

Quantum computer; QPU. A quantum computer (also “QPU” for quantum processing unit) is any information-processing device that represents and manipulates information using qubits that can exist in superposition and become entangled, and that executes quantum operations (gates, measurements, or analog evolutions) to produce classical and/or quantum outputs. Example physical implementations include, without limitation, trapped-ion, superconducting-circuit, neutral-atom, spin/defect, photonic, and annealing/Ising-type systems. Devices may be noisy, intermediate-scale systems or fault-tolerant systems, and may be general-purpose, special-purpose, or analog. QPUs may be local to the client device (110), packaged with conventional compute (e.g., 113/114), housed in a nearby accessory, or accessed remotely over the network (160).

Quantum computing; hybrid quantum-classical processing. Quantum computing denotes the execution of quantum algorithms on a QPU, optionally in a hybrid configuration where classical processors (e.g., CPU/GPU/NPU 113/114) prepare data, evaluate cost functions, perform gradient steps, or post-process measurement results. Quantum and/or hybrid workflows can run on the agent (150), the client (110), or across both using the partition policies described for 481/482.

Quantum algorithms. A quantum algorithm is any computational procedure that exploits quantum effects to estimate, search, optimize, simulate, or learn. Non-limiting classes include amplitude amplification/estimation, phase estimation, quantum Fourier transforms, quantum walks, variational algorithms (e.g., VQE/QAOA-style ansätze), quantum kernel and feature-map methods, quantum linear-systems and gradient-based solvers, and analog/Ising optimization. Algorithms can target discrete, continuous, or probabilistic objectives and may be domain-adapted to, e.g., selection logic (474), segmentation (610-615), or overlay generation (360).

Quantum chips/hardware; quantum peripherals. Quantum hardware encompasses QPUs and supporting subsystems such as cryogenic control, vacuum/laser systems, integrated photonics (sources, interferometers, detectors), microwave/RF control, error-correction controllers, and quantum peripherals including quantum random-number generators, quantum key-distribution transceivers, and coherent/Ising accelerators. Hardware may be discrete, co-packaged with 113/114, or delivered as a service reachable over 160.

Post-quantum and quantum-network security. Post-quantum cryptography (PQC) refers to classical cryptographic schemes designed to remain secure against quantum adversaries; quantum networking includes quantum-key distribution (QKD), entanglement-assisted links, and quantum-safe keying anchored by quantum randomness. These may be used with the secure transport (500) and retention controls (486/487/488).

Quantum-inspired methods. Quantum-inspired methods are classical algorithms and hardware (e.g., simulated annealing, tensor-network solvers, coherent optical Ising machines operated classically) that emulate mathematical structures of quantum approaches and may serve as fallbacks when a QPU is unavailable.

Fault-tolerant quantum computing; dynamic circuits. A fault-tolerant QPU executes deep quantum circuits under error correction with mid-circuit measurement and classical feed-forward (“dynamic circuits”) enabling real-time branches. Where referenced herein, a “QPU” includes such devices and close analogs capable of sustained, low-error executions suitable for interactive inference and optimization loops.

Quantum memory/QRAM; block-encoding. Quantum memory denotes hardware or logical facilities that allow state preparation or addressable loading of classical data into quantum states, including QRAM and/or block-encoding/state-preparation oracles that provide functionally equivalent access for similarity, search, and linear-algebra subroutines used by the agent (150) and client (110).

Quantum linear-algebra and signal-processing primitives. Quantum signal processing (QSP/QSVT), quantum linear-systems solvers, spectral transforms (e.g., QFT, phase estimation), and amplitude-amplification/estimation are building blocks the system may call to accelerate inference, ranking, segmentation, and selection.

Quantum networking. Quantum-network capabilities (e.g., QKD, entanglement distribution with repeaters, delegated/measurement-based protocols) may be available between 110↔150 and/or near-edge nodes, and can be treated as part of the secure transport (500) and partition (481/482).

Computing element (quantum-capable or classical). As used herein, a computing element is any hardware, firmware, and/or logical service that executes instructions or performs computations for the systems and methods described (e.g., the client 110, the agent 150, or nodes in 481/482). A computing element may be purely classical (e.g., CPU/GPU/NPU/DSP), quantum-capable (locally or via a network endpoint), quantum-inspired, analog, digital, or hybrids thereof. Unless expressly stated otherwise, any function attributed to a computing element may be carried out by devices having any subset of the characteristics listed below, and no single characteristic is required.

Quantum processing devices—characteristics (non-limiting). Where this document refers to a “quantum computer” or “QPU” as defined above, implementations may exhibit one or more of the following optional properties, without limitation: (i) information carriers that support superposition and/or entanglement (e.g., qubits, qudits, or continuous-variable modes); (ii) use of quantum interference to steer outcome probabilities; (iii) operation in circuit/gate model, adiabatic/annealing or analog-Hamiltonian regimes, or measurement-based quantum computation (MBQC); (iv) dynamic circuits (mid-circuit measurement with classical feed-forward); (v) error-mitigation and/or error-correction capabilities (including logical qubits); (vi) access to quantum memory or functional equivalents that prepare or encode data for quantum subroutines (e.g., state-preparation oracles, block-encoding, or QRAM-class access); (vii) physical realizations including, without limitation, superconducting circuits, trapped ions, neutral atoms, spins/defects, and photonic/optical platforms; or (viii) availability on-device, as a near-edge accessory, or remotely over the network (160), selectable by the partition policy (481/482) under the same security envelope (500). The absence of any listed property does not exclude an embodiment from scope.

Quantum algorithm families and primitives-examples (non-limiting). Where this document refers to “quantum algorithms” as defined above, examples include: (i) amplitude amplification and amplitude estimation; (ii) phase estimation and the quantum Fourier transform (QFT); (iii) quantum walks and Grover-class search; (iv) variational methods such as the Variational Quantum Eigensolver (VQE) and the Quantum Approximate Optimization Algorithm (QAOA); (v) quantum signal processing and quantum singular-value transformation (QSVT); (vi) quantum linear-systems solvers and quantum gradient/Hessian estimation; (vii) delegated or measurement-based protocols (MBQC); or (viii) hybrid quantum-classical subroutines that invoke quantum kernels within a classical loop. As used herein, “quantum memory” encompasses QRAM or functional equivalents (e.g., state-preparation oracles or block-encoding) sufficient to run such families at the stated scale. Any subset of the above may be used; classical emulations and quantum-inspired methods remain within scope.

Quantum-inspired accelerators. Devices and algorithms that emulate certain quantum mathematical structures (e.g., coherent optical/Ising machines, simulated annealing, tensor-network solvers) are within scope as computing elements even when implemented on classical hardware. They may be used interchangeably with or as fallbacks for QPUs.

Quantum endpoints and interfaces. A quantum endpoint is a local or remote interface that exposes quantum or quantum-inspired capabilities to the system (e.g., via 481/482 partitioning). Interface details (APIs/protocols, queues, compilation) are implementation-dependent and non-limiting; admission control can defer to classical paths when, e.g., latency or policy would be breached.

Non-limiting combinations; optionality. Unless otherwise stated, references to quantum devices/algorithms indicate optional capabilities of a computing element. All classical implementations remain within scope; the described quantum features may be present, absent, simulated, or replaced by functional equivalents without departing from the disclosed technology.

Computing element property clause (non-limiting). Any computing element described herein (including without limitation the client device (110), the AI agent (150), and compute targets selected by partition (481/482)) may be implemented using classical, quantum-capable, or quantum-inspired hardware or services. Such computing elements may exhibit any subset of the following properties: superposition/entanglement, interference, dynamic circuits with mid-circuit measurement, error-mitigation or error-correction, algorithmic support for amplitude estimation, phase estimation, quantum Fourier transform, quantum walks, variational optimization, quantum signal processing/singular-value transformation, quantum linear-systems or gradient estimation, or access to quantum memory or functional equivalents for state preparation or block-encoding. The absence of any listed property does not exclude an embodiment from the scope of this disclosure.

Agent-side hybrid quantum inference. In some embodiments, the agent (150) performs quantum-assisted inference by mapping pose/movement/breath features to quantum-amenable representations at request time and invoking a QPU to compute: (i) quantum kernels for similarity between the user and entries in the model/pose store (170); (ii) variational or QSVT-based classifiers that output severity and breath-sync labels; or (iii) amplitude-estimation of probabilities used for confidence and ranking. Outputs are fused with classical pipelines (151-156) and returned as segments (471-473).

Quantum-optimal DRS selection (time-critical). Formulated as a constrained selection/order problem over candidate segments (471-473) with deadlines tied to time-to-boundary (611), the agent or the client encodes a cost Hamiltonian and/or uses QAOA/QSP/QSVT and/or quantum linear-systems primitives to propose a near-optimal subset consistent with corrections-count caps (FIG. 5) and personalization weights. With fault-tolerant resources, the solver can meet sub-100 ms budgets and stream updates during ingress/egress, allowing the client DRS (250/474) to render immediately.

Quantum spatio-temporal features for phases and timing. Short windows of kinematics are block-encoded and processed with phase-estimation/QSP to yield robust spectral features that improve phase segmentation (610), boundary timing (611), and transition-duration estimates (612) under jitter and occlusion; these signals feed 156 and 474 as before.

Quantum-accelerated reference retrieval over large stores. With quantum memory (QRAM or functional equivalent), the agent performs amplitude-amplified nearest-neighbor/quantum-walk retrieval over very large, federated 170 stores to shortlist references or corrective exemplars, reducing latency before composing 472.

Training & large-scale optimization with quantum solvers. Offline and periodic updates may use quantum linear-algebra (e.g., least-squares/regression via Quantum Linear Systems Algorithm (QLSA)), gradient/Hessian estimation and quantum Monte-Carlo samplers to refine pose-deviation models and language grounding; deployed inference remains hybrid and deadline-aware.

Delegated/measurement-based agent variants. In some embodiments the agent prepares classical descriptions of circuits and delegates execution to a networked QPU over 160 using measurement-based protocols; results fold into 151-156 and 474 without exposing user identifiers, consistent with 485/500/486.

Edge QPU/photonic or superconducting accessory. In some embodiments, the client (110) integrates or pairs with a compact QPU (e.g., photonic or mini-cryogenic accessory). The app (200) routes latency-critical micro-optimizations (e.g., local re-ranking of brief items, quantum-randomness for keys, or per-pose kernel lookups) to the edge QPU, respecting thermal/battery (118) and partition policy.

Real-time local selection. For imminent transitions (small time-to-phase-boundary B), the client may submit a compact representation of candidate brief cues and constraints to its local QPU to solve a deadline-constrained selection instance; on failure or policy veto, the classical heuristic (474) runs. The presentation layer (270) then renders overlays (360).

Quantum-safe & quantum-native keys. A local quantum random-number source seeds PQC (Post-Quantum Cryptography) and/or QKD-established sessions under 500; keys bind to pseudonymous IDs (488) and observe retention (487).

Near-edge offload. When a handset QPU is unavailable, the client offloads quantum subroutines to a near-edge QPU over 160 (e.g., MEC (Multi-access Edge Computing)), with admission control to meet time-to-phase-boundary B; otherwise fall back to 474 heuristics.

Precompiled quantum assets. During idle/charging, the client precomputes pose-specific circuits (e.g., reference kernels, per-pose selection oracles) and caches them (486) for reuse, reducing compile/queue latency during live coaching.

Partition including QPU endpoints. The partition policy treats QPUs (on-device, near-edge, or cloud) as compute targets alongside 481/482 and chooses among them using queue depth, calibration state, link latency/jitter, energy, and deadline (e.g., time-to-phase-boundary B); violations trigger classical fallbacks.

Opportunistic precomputation. When idle, 110/150 generate quantum-derived templates (e.g., kernels, overlay placements) and store them under 486 for session use without altering 474 semantics.

Real-time admission control. A runtime admission controller predicts whether a quantum call can complete before the next boundary (611). If not, the DRS uses precomputed quantum assets or classical selection to guarantee timely guidance.

Quantum-resilient transport & attestation. Sessions under 500 may use PQC for key exchange and signatures, optionally combined with QKD links to derive session keys; where supported, device/QPU stacks provide attestation that circuits and binaries are untampered. Policies honor 485/486/487/488.

Provenance with quantum randomness. Segments (471-473) and overlays (360) can be tagged with quantum-seeded nonces and verifiable logs to strengthen audit without revealing identity.

Delegated/“blind” quantum execution. Where supported, the client or agent uses protocols that conceal circuit intent and/or inputs from the executing QPU while still verifying results, integrating with 500 and 486 policies.

### REFERENCE NUMERALS

10: client application (overall UI).
11: a pose/action drop-down menu.
12: main menu button.
14: main menu panel.
16: poses/asanas option.
18: add-a-pose option.
20: settings option.
22: how-to/help option.
24: reference pose selector.
26: gallery comparison control.
28: live camera comparison control.
30: API key field.
32: corrections-count control.
34: image/album picker.
36: reference pose image.
38: user pose image.
40: generated instructions panel.
42: camera viewfinder.
44: camera selection button.
46: brief guidance panel.
110: client/computing device (group).
111: display.
113: processor (CPU/GPU).
114: neural processing unit (NPU).
115: image signal processor (ISP).
116: memory.
117: non-volatile storage.
118: power management integrated circuit (PMIC)/battery.
119: RF/baseband.
120: camera(s).
121: RGB camera.
122: depth camera.
123: IR camera.
124: LiDAR sensor.
125: time-of-flight sensor.
126: thermal camera.
127: sensor fusion module.
130: microphone.
140: speaker.
145: agent input(s) from client.
160: network.
165: intermediary server.
150: AI agent.
151: pose estimation.
152: movement analysis.
153: breathing analysis.
154: error classification.
155: response segmenter.
156: feedback generator.
159: agent outputs.
170: reference pose/model store.
180: wearable sensors.
190: AR/VR display or glasses.
191: world coordinate frame.
193: body coordinate frame.
192: device coordinate frame.
195: external/drone camera.
200: client application (software group).
210: capture module.
220: pre-processing module.
230: request generator.
240: state monitor.
250: dynamic response selector (DRS).
260: response processor.
270: presentation layer.
301: user pose image/frame.
310: keypoint/feature detection.
320: angle measurement.
330: deviation computation.
340: thresholding/labeling.
350: instruction generator.
360: overlay renderer.
400: mode selection.
471: brief commands segment.
472: detailed instruction segment.
473: additional information segment.
474: selection logic.
481: edge/on-device processing.
482: cloud processing.
485: anonymization/redaction.
486: compression/caching.
487: retention/delete controls.
488: pseudonymous session IDs.
500: encryption/secure transport.
600: response arrival trigger.
610: phase segmentation.
611: phase boundary detection.
612: transition duration measurement.
613: reference timing comparison.
614: breathing synchronization check.
615: severity classification.
620: quality/context issue detection.
621: new capture trigger.
622: integration of additional data.
623: data fusion.
TX in the figures stands for Transmit / Transmission / Transfer

1. A client device configured to execute a client application, the client device comprising at least one processor and a memory storing instructions that, when executed, cause the client device to:
(a) capture video depicting a user;
(b) form a request including media representing the user;
(c) cause generation of guidance from the media by at least one of: (i) executing analysis instructions on the device, or (ii) transmitting the request over an encrypted channel to a remote service that processes the media;
(d) obtain a multi-segment response that includes different levels of guidance;
(e) apply, on the device, a Dynamic Response Selector (DRS) that selects one or more segments of the multi-segment response based on an application state of the client application; and
(f) present the selected segment(s) by at least one of text, audio, or visual overlays on a display.

2. The client device of claim 1, wherein the different levels of guidance comprise at least two of the following: brief commands, detailed instructions, or additional information; and wherein the request includes a correction count that limits the number of concurrently presented brief commands and/or detailed instructions.

3. The client device of claim 1, wherein the application state comprises at least one of the following: practicing, hold, review, or idle/explore; and wherein the DRS is configured to prioritize brief commands during practicing and elevate detailed instructions during hold or review.

4. The client device of claim 1, wherein the multi-segment response includes overlay directives and the client device is configured to resolve the overlay directives into device-coordinate frames for presentation on the client device.

5. The client device of claim 1, wherein the client device is configured to retrieve a selected reference image or video clip from a model/pose store accessible to the client device and to include the selected reference in the request; and wherein the guidance includes evaluation of the user relative to the selected reference.

6. The client device of claim 1, wherein the multi-segment response is generated without emitting an explicit list of body keypoints to the client device.

7. The client device of claim 1, wherein the client device is configured to perform on-device anonymization or redaction of the media prior to transmission.

8. The client device of claim 1, wherein the client device is configured to establish a persistent, full-duplex session to continuously stream the video to the remote service and receive rolling multi-segment responses that the DRS is configured to consume upon arrival.

9. The client device of claim 1, further comprising multiple cameras and a fusion module configured to combine at least two modalities selected from RGB, depth, infrared, LiDAR, time-of-flight, or thermal and to include fused features in the request.

10. The client device of claim 1, wherein latency-sensitive text-to-speech and overlay rendering execute on the client device while model-heavy analysis executes remotely according to an edge/cloud partition policy.

11. A method performed by a client device configured to execute a client application, the method comprising:
(a) capturing video depicting a user;
(b) forming a request including media representing the user;
(c) causing generation of guidance from the media by at least one of: (i) executing analysis instructions on the client device, or (ii) transmitting the request over an encrypted channel to a remote service that processes the media;
(d) obtaining a multi-segment response that includes different types of guidance;
(e) selecting, by a Dynamic Response Selector (DRS) on the client device based on an application state of the client application, one or more segments of the multi-segment response; and
(f) presenting the selected segment(s) by at least one of text, audio, or image.

12. The method of claim 11, further comprising retrieving a reference image or video clip from a model/pose store accessible to the client device and including the selected reference in the request; wherein the guidance includes evaluation of the user relative to the selected reference.

13. The method of claim 11, further comprising establishing a streaming session in which the client device continuously streams the video and audio to the remote service and receives rolling multi-segment responses that are consumed as they arrive.

14. The method of claim 11, wherein the obtained response lacks an explicit list of body keypoints.

15. The method of claim 11, further comprising, in response to a quality/context condition detected by the client device, triggering additional capture, integrating additional data, and fusing modalities before causing generation of guidance.

16. A non-transitory computer-readable storage medium storing instructions that, when executed by one or more processors of a client device, cause the client device to:
(a) capture video depicting a user;
(b) form a request including media representing the user;
(c) obtain a multi-segment response that includes different types of guidance; and
(d) select, by a Dynamic Response Selector based on an application state of a client application on the device, one or more segments of the multi-segment response for presentation by at least one of text, image, or audio.

17. The storage medium of claim 16, wherein different instruction sets are stored at different times to provide the operations of (a)-(d).

18. The storage medium of claim 16, wherein the instructions further cause the device to retrieve a reference image or video clip from a model/pose store accessible to the client device and to include the selected reference in the request.

19. The storage medium of claim 16, wherein the instructions further cause the device to establish a persistent, streaming session and to consume rolling multi-segment responses as they arrive.

20. The storage medium of claim 16, wherein the instructions further cause the DRS to use at least one of the following: phase segmentation, phase-boundary timing, transition duration, breathing synchronization, or severity classifications to schedule presentation with a hysteresis policy near phase boundaries.
